(19) United States
(12) Patent Application Publication
(10) Pub. No.: US 2003/0099994 A1
(43) Pub. Date: May 29, 2003

(54) Novel FDRG protein and nucleic acid molecules and uses therefor

(75) Inventors: Douglas A. Holtzman, Cambridge, MA (US); Bruce M. Spiegelman, Waban, MA (US); Clifford Hyunsuk Yoon, Cambridge, MA (US)

Correspondence Address: LAHIVE & COCKFIELD, 28 STATE STREET, BOSTON, MA, 02109, US

(73) Assignee: Millennium Pharamceuticals, Inc.

(21) Appl. No.: 10/263,677

(22) Filed: Oct. 3, 2002

Related U.S. Application Data
(63) Continuation of application No. 09/546,547, filed on Apr. 7, 2000, now abandoned.
Application No. 09/546,547 is a continuation-in-part of application No. 09/261,004, filed on Mar. 2, 1999.
Application No. 09/261,004 is a continuation-in-part of application No. 09/033,539, filed on Mar. 2, 1998, now abandoned.

(51) Int. Cl. C12Q 1/68; G01N 33/53; C07H 21/04; C12P 21/02; C12N 5/06; C07K 14/485

(52) U.S. Cl. 435/6; 435/7.1; 536/23.5; 530/399; 435/69.1; 435/320.1; 435/325

(57) ABSTRACT

Novel FDRG polypeptides, proteins, and nucleic acid molecules are disclosed. In addition to isolated, full-length FDRG proteins, the invention further provides isolated FDRG fusion proteins, antigenic peptides and anti-FDRG antibodies. The invention also provides FDRG nucleic acid molecules, recombinant expression vectors containing a nucleic acid molecule of the invention, host cells into which the expression vectors have been introduced and non-human transgenic animals in which a FDRG gene has been introduced or disrupted. Diagnostic, screening and therapeutic methods utilizing compositions of the invention are also provided.

### CROSS-REFERENCE TO RELATED APPLICATIONS

The present application is a continuation-in-part of U.S. patent application Ser. No. 09/261,004 entitled “Novel FDRG Protein and Nucleic Acid Molecules and Uses Therefor” (filed Mar. 2, 1999), which is a continuation-in-part of U.S. patent application Ser. No. 09/033,539 entitled “Novel FDRG Protein and Nucleic Acid Molecules and Uses Therefor” (filed Mar. 2, 1998). The content of the above-referenced patent applications is incorporated herein by this reference in its entirety.

### GOVERNMENT FUNDING

This invention was made in part with government support under USPHS Grant DK31405, awarded by the National Institutes of Health. The government may have certain rights in this invention.

### BACKGROUND OF THE INVENTION

Blood vessel formation occurs by two processes, vasculogenesis and angiogenesis. Vasculogenesis involves establishment of a primitive vascular network during embryogenesis. Subsequently, angiogenesis remodels this network to form the mature cardiovascular system. In addition, angiogenesis takes place in adult tissues during tissue repair, the reshaping of female reproductive tissues and tumor growth and metastasis. (Maisonpierre et al. (1997) Science 277:55-60).

Endothelial cells have been found to be centrally involved in both vasculogenesis and angiogenesis. These cells migrate, proliferate and assemble to form tubes with tight cell to cell connections. Peri-endothelial support cells are then recruited to encase the endothelial tube to thereby provide maintenance and modulary functions to the blood vessels. (Hanahan (1997) Science 277:48-50).

The process of establishing and remodeling of blood vessels is controlled by the interaction of several protein ligands and tyrosine kinase-like receptors expressed on endothelial cells. Such receptors include, for example, the recently discovered TIE receptor family consisting of TIE1 and TIE2 which have been found to be critically involved in the formation of vasculature. Dumont et al. (1994) Genes Dev. 8:1897-1909; Sato et al. (1995) Nature 376:70-74. Moreover, ligands which bind to the TIE2 receptor and belong to the protein family which includes fibrinogen and ficolin have been discovered. These ligands include angiopoietin-1 which binds and induces phosphorylation of TIE2 (Davis et al. (1996) Cell 87:1161-1169) and angiopoietin-2 which acts as a natural antagonist for angiopoietin-1 and TIE2 (Maisonpierre (1997) Science 277:55-60). Both of these ligands, angiopoietin-1 and angiopoietin-2, have been implicated in blood vessel formation and remodeling.

Recently, an interesting connection was made between the divergent fields of angiogenesis and obesity. In particular, it was demonstrated that leptin, a protein secreted by fat cells, functions as both a metabolic hormone and a bonafide angiogenic factor (Sierra-Honigmann et al. (1998) Science 281:1683-1686). The finding that a factor secreted by fat cells having a known role in metabolism and obesity also plays a role in angiogenesis demonstrates a novel link between adipocytes and the vasculature. The complex functionality of leptin raises the possibility that there exist other molecules which may play a dual role in both angiogenesis and obesity. Accordingly, there exists a need to identify and characterize such molecules and to understand the mechanism by which organisms utilize one factor in controlling multiple cellular functions. Moreover, there exists a need for identifying molecules with which such dual regulators interact as well as identifying modulators of such dual regulators.

### SUMMARY OF THE INVENTION

The present invention is based, at least in part, on the discovery of novel secreted molecules having homology to angiopoietin which are believed to modulate angiogenesis and mediate the contribution of adipocytes to various metabolic processes. These novel molecules are referred to herein as Fibrinogen Domain Related (referred to herein as “FDRG” or TANGO 115) nucleic acid and protein molecules and are useful as modulating agents in regulating a variety of cellular processes (e.g., angiogenesis-related and metabolism-related processes). Accordingly, in one aspect, this invention provides isolated nucleic acid molecules encoding FDRG proteins or biologically active portions thereof, as well as nucleic acid fragments suitable as primers or hybridization probes for the detection of FDRG-encoding nucleic acids.

In one embodiment, a FDRG nucleic acid molecule is 60% homologous to the nucleotide sequence shown in SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a complement thereof. In another embodiment, an isolated FDRG nucleic acid molecule encodes the amino acid sequence of human FDRG shown in SEQ ID NO:2. In another embodiment, an isolated FDRG nucleic acid molecule encodes the amino acid sequence of murine FDRG shown in SEQ ID NO:12.

In a preferred embodiment, an isolated FDRG nucleic acid molecule has the nucleotide sequence of SEQ ID NO:1, or a complement thereof. In another embodiment, a FDRG nucleic acid molecule further comprises nucleotides 1-165 of SEQ ID NO:1. In another embodiment, a FDRG nucleic acid molecule further comprises nucleotides 1384-1894 of SEQ ID NO:1. In yet another embodiment, a FDRG nucleic acid molecule comprises the nucleotide sequence of SEQ ID NO:3.

In another preferred embodiment, an isolated FDRG nucleic acid molecule has the nucleotide sequence of SEQ ID NO:11, or a complement thereof. In another embodiment, a FDRG nucleic acid molecule further comprises nucleotides 1-148 of SEQ ID NO:11. In yet another embodiment, a FDRG nucleic acid molecule further comprises nucleotides 1379-1893 of SEQ ID NO:11. In yet another embodiment, a FDRG nucleic acid molecule comprises the nucleotide sequence of SEQ ID NO:13.

In another preferred embodiment of the invention, an isolated FDRG nucleic acid molecule has the nucleotide sequence of SEQ ID NO:3, SEQ ID NO:4, SEQ ID NO:13, SEQ ID NO:14 or a complement thereof. In yet another embodiment, a FDRG nucleic acid molecule comprises nucleotides 1-556 of SEQ ID NO:3 or SEQ ID NO:13 or nucleotides 1-481 of SEQ ID NO:4 or SEQ ID NO:14.

In yet another preferred embodiment, an isolated FDRG nucleic acid molecule has the nucleotide sequence of the DNA insert of the plasmid deposited with ATCC as Accession Number 98632, or a complement thereof.

In another embodiment, a FDRG nucleic acid molecule includes a nucleotide sequence encoding a protein having an amino acid sequence sufficiently homologous to the amino acid sequence of SEQ ID NO:2, SEQ ID NO:5, SEQ ID NO:12, or SEQ ID NO:15. In yet another embodiment, a FDRG nucleic acid molecule includes a nucleotide sequence encoding a protein having an amino acid sequence at least 60% homologous to the amino acid sequence of SEQ ID NO:2, SEQ ID NO:5, SEQ ID NO:12, or SEQ ID NO:15. In a preferred embodiment, a FDRG nucleic acid molecule includes a nucleotide sequence encoding a protein having the amino acid sequence of SEQ ID NO:2, SEQ ID NO:5, SEQ ID NO:12, or SEQ ID NO:15.

In another embodiment, an isolated nucleic acid molecule of the present invention encodes a FDRG protein which includes a signal sequence, an N-terminal unique domain, a C-terminal fibrinogen-like domain, and is secreted. In another embodiment, the FDRG nucleic acid molecule encodes a FDRG protein and is a naturally occurring nucleotide sequence. In yet another embodiment, an isolated nucleic acid molecule of the present invention encodes a FDRG protein and comprises a nucleotide sequence which hybridizes under stringent hybridization conditions to a nucleic acid molecule having the nucleotide sequence of SEQ ID NO:1, SEQ ID NO:3, SEQ ID NO:4, SEQ ID NO:11, SEQ ID NO:13, SEQ ID NO:14, or of the DNA insert of the plasmid deposited with ATCC as Accession Number 98632.

Another embodiment of the invention features FDRG nucleic acid molecules which specifically detect FDRG nucleic acid molecules relative to nucleic acid molecules encoding non-FDRG proteins. For example, in one embodiment, a FDRG nucleic acid molecule hybridizes under stringent conditions to a nucleic acid molecule comprising nucleotides 1-721 of SEQ ID NO:1 or SEQ ID NO:11. In another exemplary embodiment, a FDRG nucleic acid molecule hybridizes under stringent conditions to a nucleic acid molecule comprising nucleotides 1-556 of SEQ ID NO:3 or SEQ ID NO:13. In another embodiment, a FDRG nucleic acid molecule hybridizes under stringent conditions to a nucleic acid molecule comprising nucleotides 1-481 of SEQ ID NO:4 or SEQ ID NO:14. In another embodiment, the FDRG nucleic acid molecule is at least 500 to 550 nucleotides in length, 600 to 650 nucleotides in length, 700 to 750 nucleotides in length, 800 to 850 nucleotides in length and hybridizes under stringent conditions to a nucleic acid molecule comprising the nucleotide sequence shown in SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a complement thereof Another embodiment the invention provides an isolated nucleic acid molecule which is antisense to the coding strand of a FDRG nucleic acid.

Another aspect of the invention provides a vector comprising a FDRG nucleic acid molecule. In certain embodiments, the vector is a recombinant expression vector. In another embodiment the invention provides a host cell containing a vector of the invention. The invention also provides a method for producing FDRG protein by culturing in a suitable medium, a host cell of the invention containing a recombinant expression vector such that FDRG protein is produced.

Another aspect of this invention features isolated or recombinant FDRG proteins and polypeptides. In one embodiment, an isolated FDRG protein has a signal sequence, an N-terminal unique domain, a C-terminal fibrinogen-like domain, and is secreted. In another embodiment, an isolated FDRG protein has an N-terminal unique domain and a C-terminal fibrinogen-like domain.

In another embodiment, an isolated FDRG protein has an amino acid sequence sufficiently homologous to the amino acid sequence of SEQ ID NO:2, SEQ ID NO:5, SEQ ID NO:12 or SEQ ID NO:15. In a preferred embodiment, a FDRG protein has an amino acid sequence at least about 60% homologous to the amino acid sequence of SEQ ID NO:2, SEQ ID NO:5, SEQ ID NO:12 or SEQ ID NO:15. In another embodiment, a FDRG protein has the amino acid sequence of SEQ ID NO:2, SEQ ID NO:5, SEQ ID NO:12 or SEQ ID NO:15.

Another embodiment of the invention features an isolated FDRG protein which is encoded by a nucleic acid molecule having a nucleotide sequence at least about 60% homologous to a nucleotide sequence of SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632, or a complement thereof. This invention also features an isolated FDRG protein which is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a complement thereof. In another embodiment, the polypeptide is fragment of a FDRG polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632, where the fragment comprises at least 15 contiguous amino acids of SEQ ID NO:2, SEQ ID NO:5, SEQ ID NO:12, SEQ ID NO:15, or the amino acid sequence encoded by the DNA insert of the plasmid deposited with ATCC as Accession Number 98632

The FDRG proteins of the present invention, or biologically active portions thereof, can be operatively linked to a non-FDRG polypeptide to form FDRG fusion proteins. The invention further features antibodies that specifically bind FDRG proteins, such as monoclonal or polyclonal antibodies. In addition, the FDRG proteins or biologically active portions thereof can be incorporated into pharmaceutical compositions, which optionally include pharmaceutically acceptable carriers.

In another aspect, the present invention provides a method for detecting FDRG expression in a biological sample by contacting the biological sample with an agent capable of detecting a FDRG nucleic acid molecule, protein or polypeptide such that the presence of FDRG nucleic acid molecule, protein or polypeptide is detected in the biological sample.

In another aspect, the present invention provides a method for detecting the presence of FDRG activity in a biological sample by contacting the biological sample with an agent capable of detecting an indicator of FDRG activity such that the presence of FDRG activity is detected in the biological sample.

In another aspect, the invention provides a method for modulating FDRG activity comprising contacting the cell with an agent that modulates FDRG activity such that FDRG activity in the cell is modulated. In one embodiment, the agent inhibits FDRG activity. In another embodiment, the agent stimulates FDRG activity. In one embodiment, the agent is an antibody that specifically binds to FDRG protein. In another embodiment, the agent modulates expression of FDRG by modulating transcription of a FDRG gene or translation of a FDRG mRNA. In yet another embodiment, the agent is a nucleic acid molecule having a nucleotide sequence that is antisense to the coding strand of the FDRG mRNA or the FDRG gene.

In one embodiment, the methods of the present invention are used to treat a subject having a disorder characterized by aberrant FDRG protein or nucleic acid expression or activity by administering an agent which is a FDRG modulator to the subject. In one embodiment, the FDRG modulator is a FDRG protein. In another embodiment, the FDRG modulator is a FDRG nucleic acid molecule. In yet another embodiment, the FDRG modulator is a peptide, peptidomimetic, or other small molecule. In a preferred embodiment, the disorder characterized by aberrant FDRG protein or nucleic acid expression is a hematopoietic disorder, a differentiative or developmental disorder, a proliferative disorder or a cell recruitment disorder.

The present invention also provides a diagnostic assay for identifying the presence or absence of a genetic alteration characterized by at least one of (i) aberrant modification or mutation of a gene encoding a FDRG protein; (ii) mis-regulation of said gene; and (iii) aberrant post-translational modification of a FDRG protein, wherein a wild-type form of said gene encodes an protein with a FDRG activity.

In another aspect the invention provides a method for identifying a compound that binds to or modulates the activity of a FDRG protein, by providing an indicator composition comprising a FDRG protein having FDRG activity, contacting the indicator composition with a test compound, and determining the effect of the test compound on FDRG activity in the indicator composition to identify a compound that modulates the activity of a FDRG protein.

In another aspect, the invention provides a method of treating obesity or diabetes in a subject which involves administering to the subject an agent (e.g., a small molecule modulator of FDRG, a FDRG nucleic acid molecule, or a FDRG antibody). In yet another aspect, the invention provides a method for diagnosing obesity or diabetes in a subject utilizing FDRG nucleic acid molecles. In yet another aspect, the invention provides a method for identifying compounds which modulate metabolic disorders utilizing cells which expresses a FDRG receptor. In yet another aspect, the invention provides a method of regulating angiogenesis in a subject.

Other features and advantages of the invention will be apparent from the following detailed description and claims.

### BRIEF DESCRIPTION OF THE DRAWINGS

FIG. 1 depicts the cDNA sequence and predicted amino acid sequences of human FDRG (also referred to as “hFDRG” or “hT115”). The nucleotide sequence corresponds to nucleic acids 1 to 1894 of SEQ ID NO:1. The amino acid sequence corresponds to amino acids 1 to 406 of SEQ ID NO:2.

FIG. 2 depicts the cDNA sequence and predicted amino acid sequence of mature human FDRG. The nucleotide sequence corresponds to nucleic acids 1 to 1143 of SEQ ID NO:4. The amino acid sequence corresponds to amino acids 1 to 381 of SEQ ID NO:5.

FIG. 3 depicts a multiple sequence alignment of the amino acid sequences of human FDRG (corresponding to amino acids 1 to 406 of SEQ ID NO:2), murine FDRG (corresponding to amino acids 1 to 410 of SEQ ID NO:12), human angiopoietin 2 (Genbank™ Accession No. AF004327) (SEQ ID NO:8), murine angiopoietin 2 (Genbank™ Accession No. AF004326) (SEQ ID NO:9), and porcine ficolin (Genbank™ Accession No. L 12345) (SEQ ID NO:10). The alignment was generated using MegAlign™ sequence alignment software. The initial pairwise alignment step was performed using a Lipman Pearson algorithm with a K-tuple of 1, a GAP penalty of 3, a window of 5, and diagonals saved set to 5. The multiple alignment step was performed using the Clustal algorithm with a PAM 250 residue weight Table, a GAP penalty of 10, and a GAP length penalty of 10.

FIG. 4 depicts the cDNA sequence and predicted amino acid sequences of murine FDRG (also referred to as “mFDRG”, “mT115”, or BK89). The nucleotide sequence corresponds to nucleic acids 1 to 1893 of SEQ ID NO:11. The amino acid sequence corresponds to amino acids 1 to 410 of SEQ ID NO:12.

FIG. 5 depicts the cDNA sequence and predicted amino acid sequence of mature murine FDRG. The nucleotide sequence corresponds to nucleic acids 1 to 1161 of SEQ ID NO:14. The amino acid sequence corresponds to amino acids 1 to 387 of SEQ ID NO:15.

FIG. 6 depicts a pairwise alignment of human and murine FDRG nucleotide sequences (corresponding to SEQ ID NO:1 and SEQ ID NO:11, respectively). The alignment was generated using the ALIGN program, version 2.0 (Myers and Miller (1989) CABIOS), scoring matrix=PAM 120, gap penalties: −12/−4.

FIG. 7 depicts a pairwise alignment of human and murine FRDG amino acid sequences (corresponding to SEQ ID NO:2 and SEQ ID NO:12, respectively). The alignment was generated as described in the legend of FIG. 6. Panel A depicts the deduced amino acid sequences of mouse and human FDRG. Bold italics indicate the limits of the coiled-coil domain and bold indicates the limits of the fibrinogen-like domains. Panel B is a schematic diagram of the predicted FDRG protein structure. Conserved cysteines are indicated by asterisks. Panel C is a schematic depiction of the genomic structure of murine FDRG. Exons are denoted by black boxes and introns by a solid line.

FIG. 8 depicts the genomic sequence of muring FDRG.

### DETAILED DESCRIPTION OF THE INVENTION

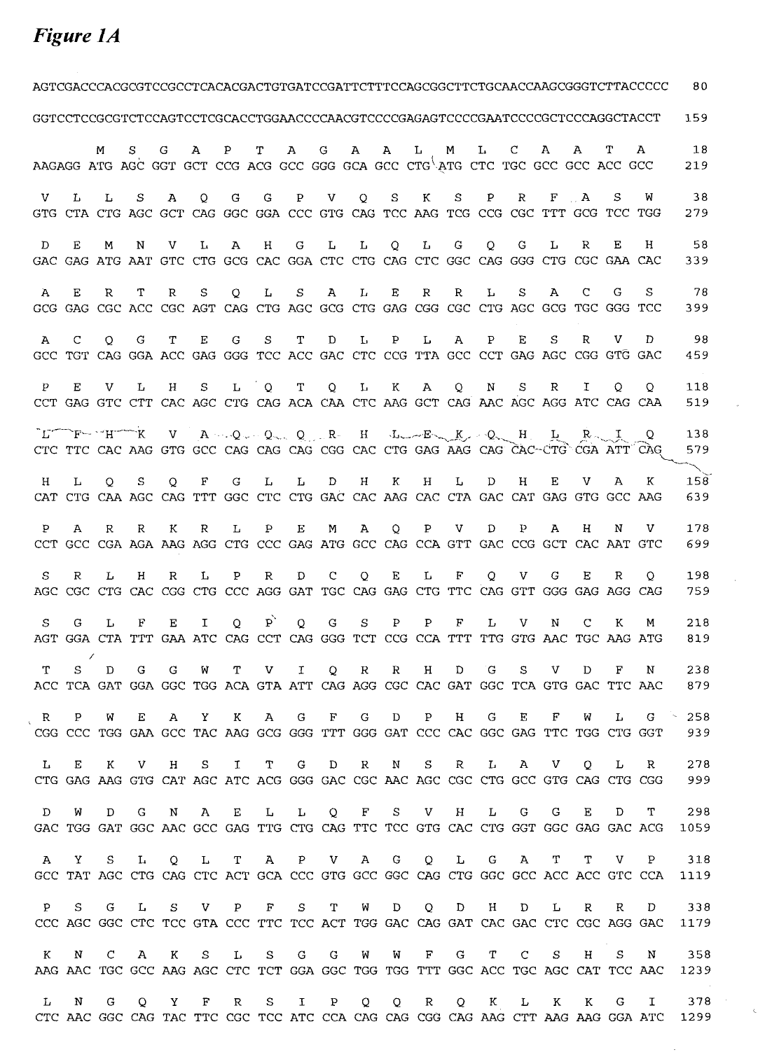

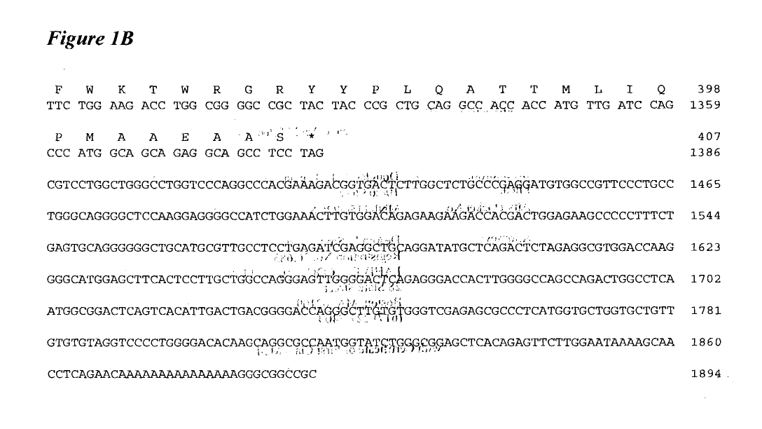

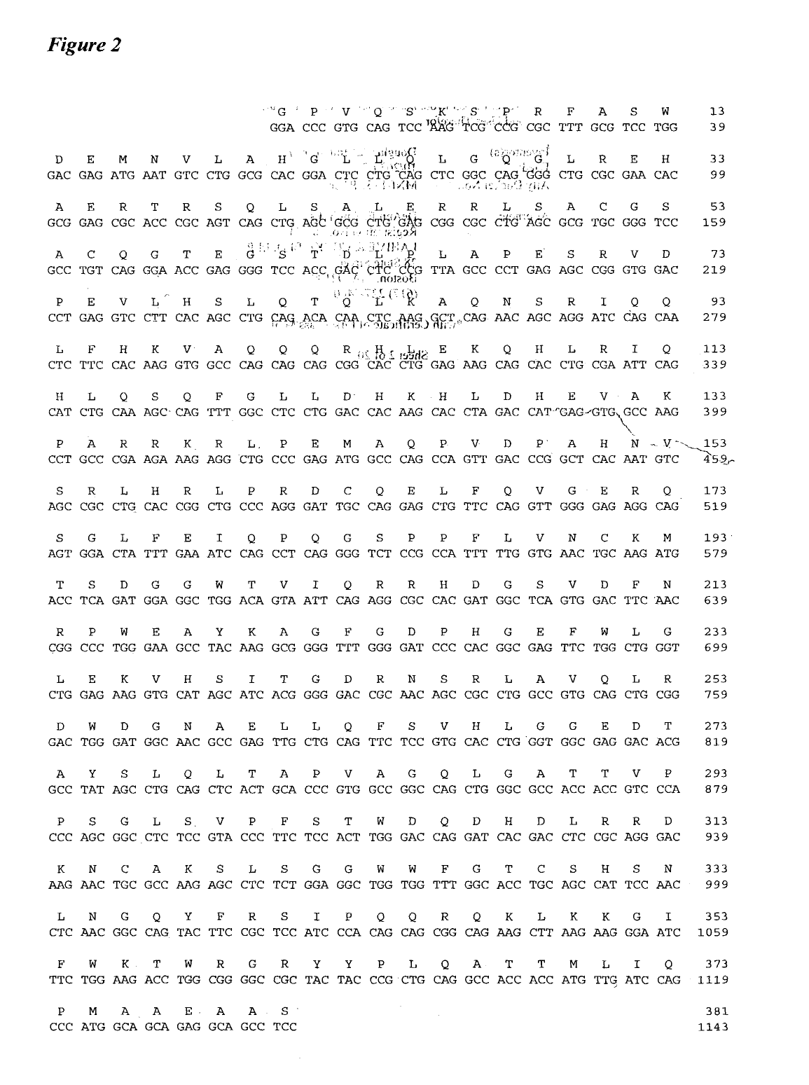

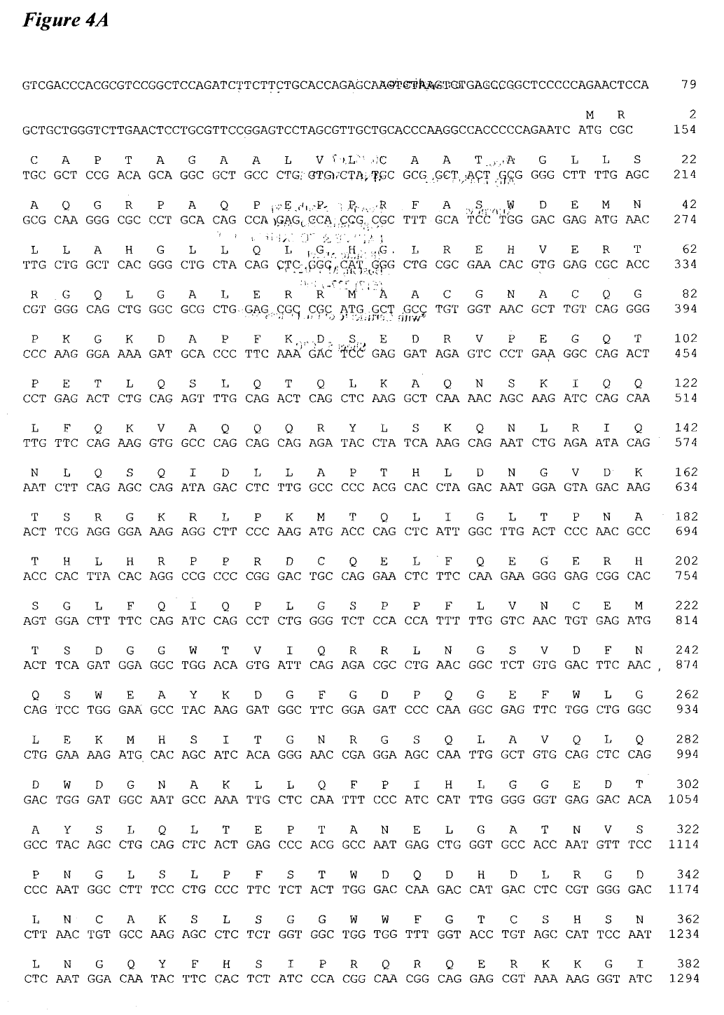

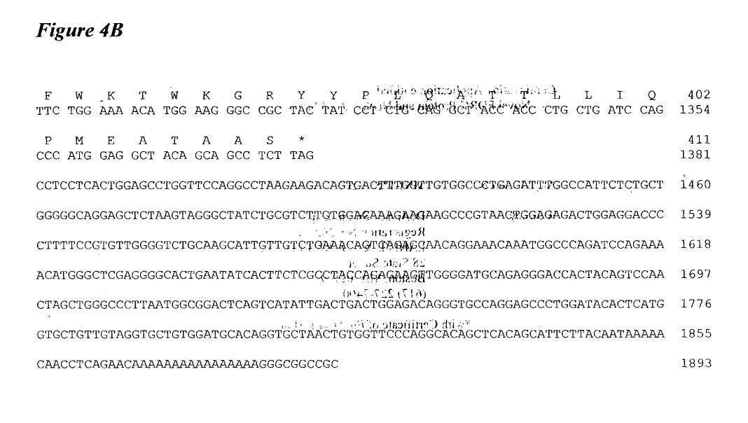

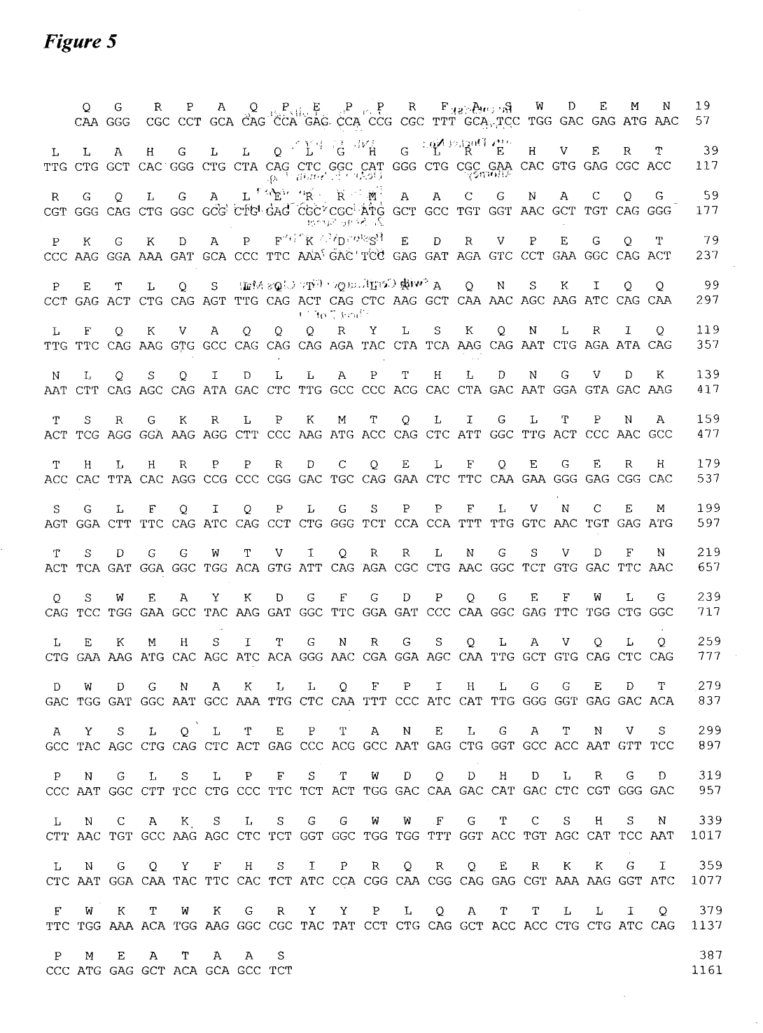

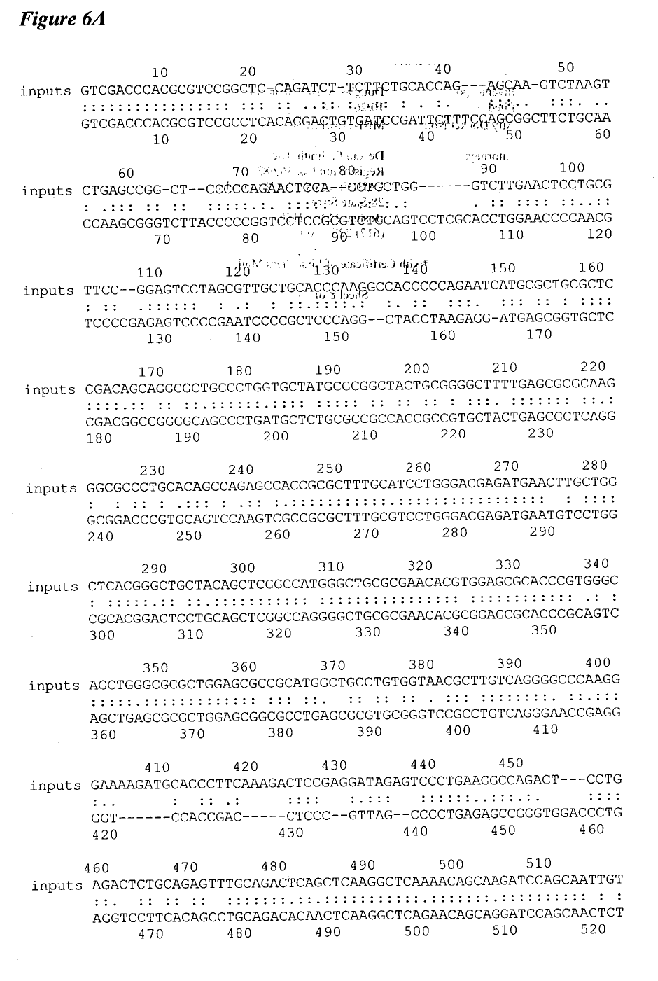

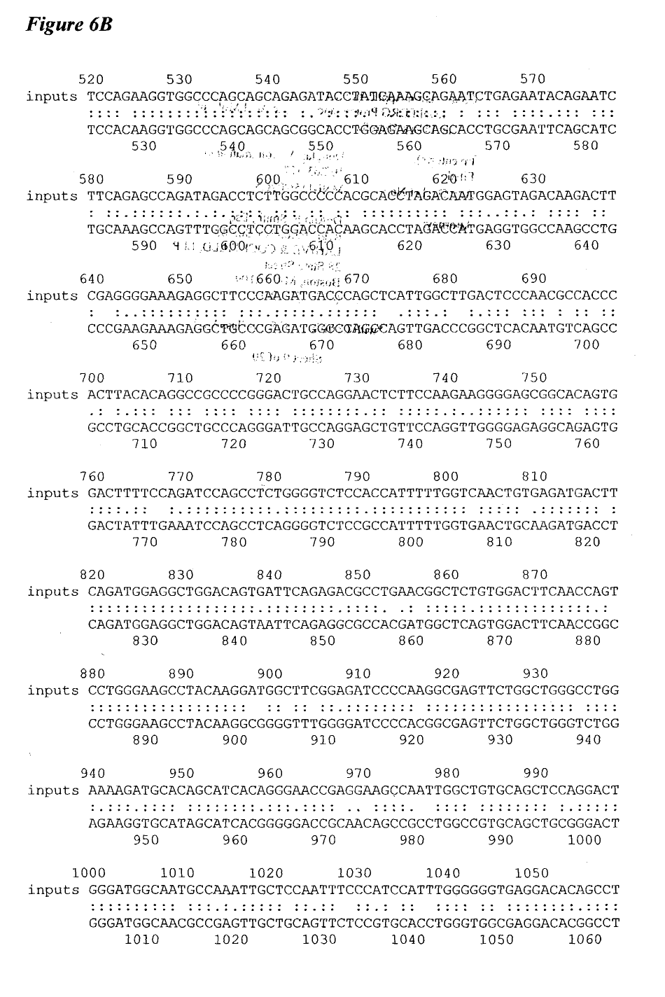

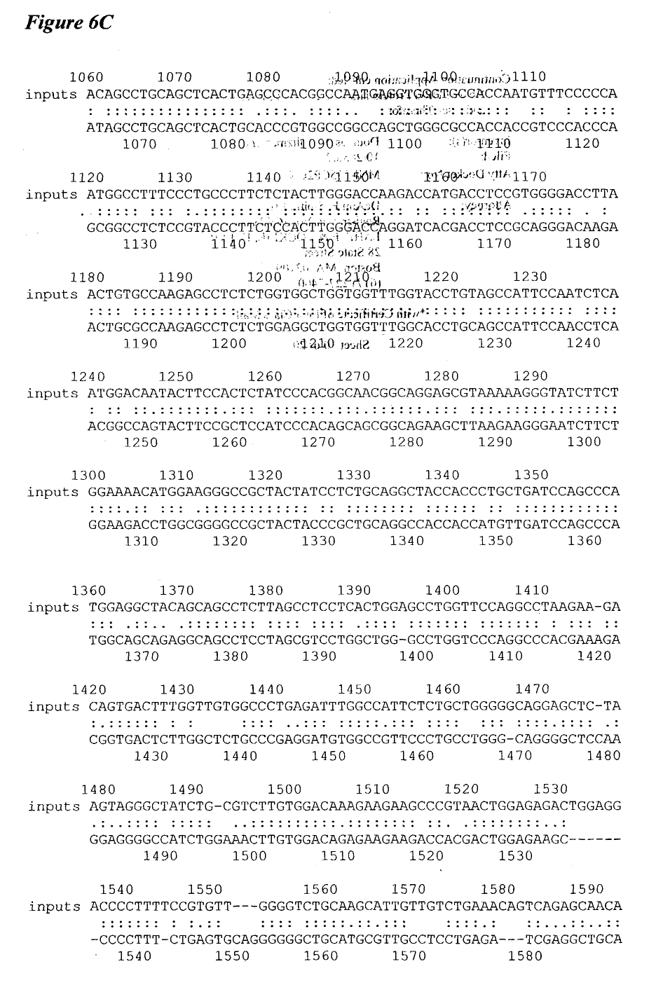

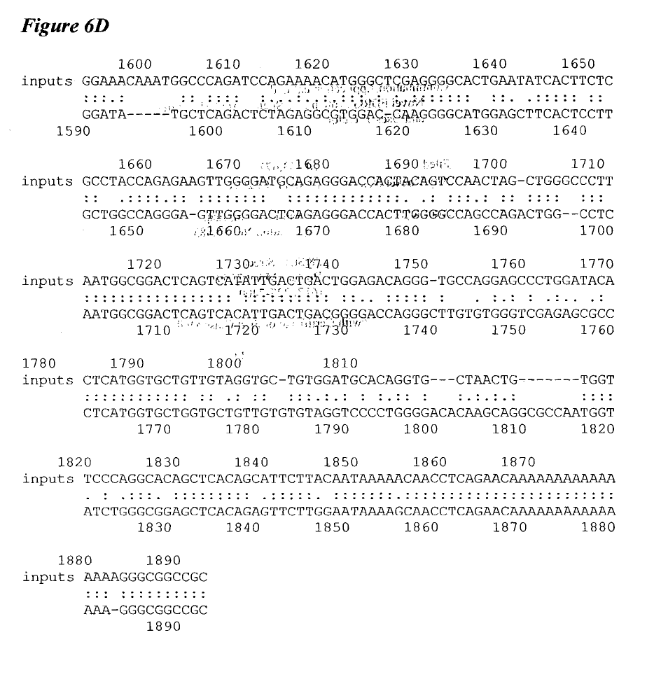

The present invention is based on the discovery of novel molecules having homology to the protein family that includes fibrinogen, angiopoietin(s), and ficolin, referred to herein as FDRG protein and nucleic acid molecules, which comprise a family of molecules having certain conserved structural and functional features. The nucleotide sequence of a human FDRG nucleic acid molecule and the amino acid sequence of the predicted human FDRG protein molecule are depicted in FIG. 1. The nucleotide sequence of a murine FDRG nucleic acid molecule and the amino acid sequence of the predicted murine FDRG protein molecule are depicted in FIG. 4.

The term “family” when referring to the protein and nucleic acid molecules of the invention is intended to mean two or more protein or nucleic acid molecules having a common structural domain and having sufficient amino acid or nucleotide sequence homology as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics. An exemplary family of the present invention is the fibrinogen-like protein family whose members include, at least, human angiopoietin 1, human angiopoietin 2, ficolin and fibrinogen. Another exemplary family of the present invention is the FDRG protein family.

In another embodiment, a FDRG family member is identified based on the presence of an “N-terminal coiled-coil domain” in the protein or corresponding nucleic acid molecule. As used herein, the term “N-terminal coiled-coil domain” includes a protein domain which is at about 100 amino acid residues in length and has a primary structure that is predicted to form a coiled-coil quaternary structure. In another embodiment, an “N-terminal coiled-coil domain” is at least about 90-110 amino acid residues in length, and preferably at least about 80-120 amino acid residues in length, and has a primary structure that is predicted to form a coiled-coil quaternary structure. In yet another embodiment, an “N-terminal coiled-coil domain” refers to a protein domain which is at about 100 amino acid residues in length, preferably at least about 90-110 amino acid residues in length, and more preferably at least about 80-120 amino acid residues in length, and has at least about 70-75%, preferably at least about 80-85%, more preferably at least about 90-95% homology, and even more preferably greater than 95% homology with amino acid residues 99-170 of SEQ ID NO:2. In yet another embodiment, an “N-terminal coiled-coil domain” consists of about amino acid residues 99-170 of SEQ ID NO:2. In yet another embodiment, an “N-terminal coiled-coil domain” consists of about amino acid residues 103-174 of SEQ ID NO:12. In a preferred embodiment, an “N-terminal coiled-coil domain” is similar to that found in other members of the fibrinogen-like protein family.

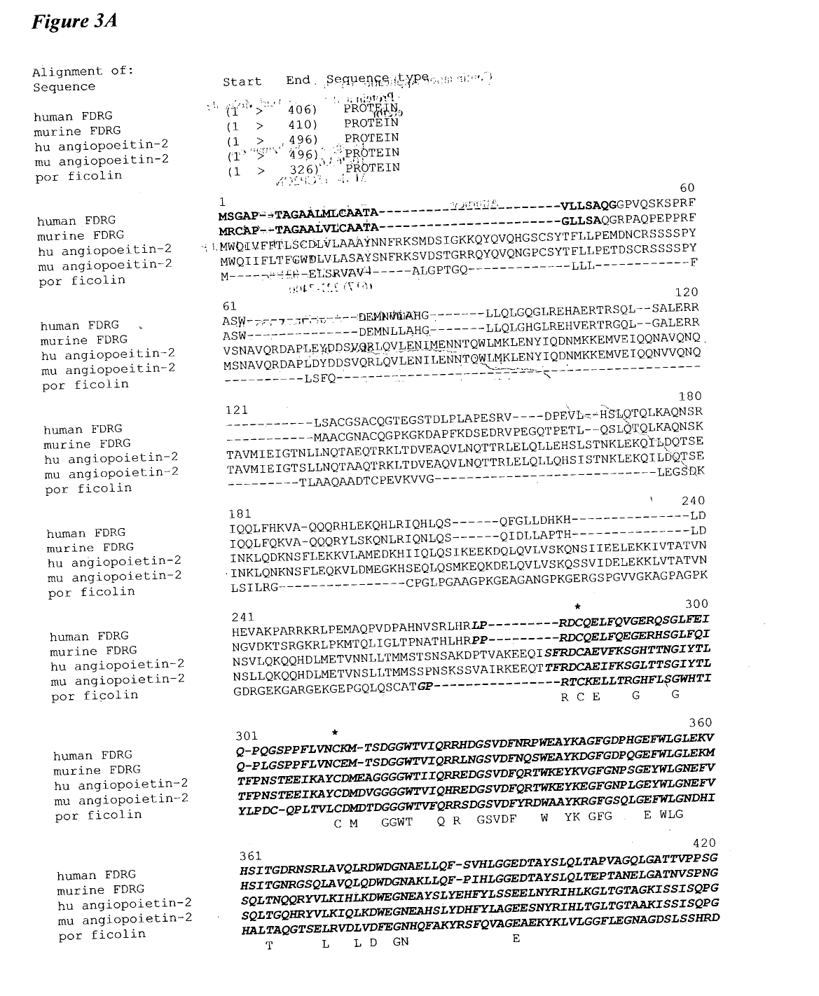

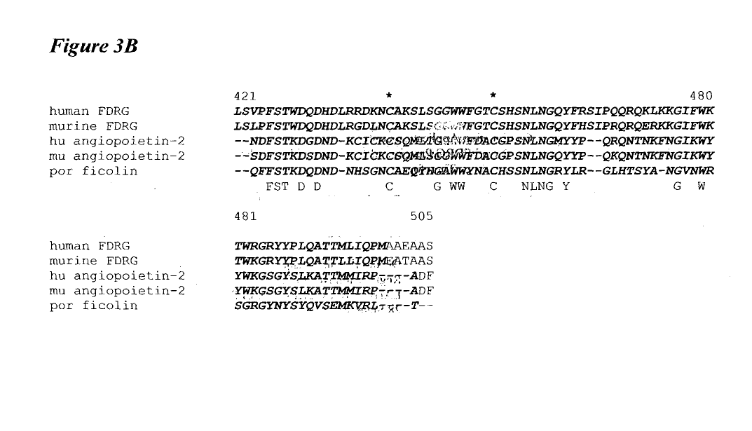

To identify the presence of a “C-terminal fibrinogen-like domain” in a FDRG family member, the amino acid sequence of the protein family member can be searched against a database of HMMs (e.g., the Pfam database, release 3.3) e.g., using the default parameters. For example, the search can be performed using the hmmsf program (family specific) and threshold score of 15 for determining a hit. hmmsf is available as part of the HMMER package of search programs (HMMER 2.1.1, December 1998) which is freely distributed by the Washington University school of medicine. In one embodiment, a hit to a fibrinogen_C (fibrinogen beta and gamma chains, C-terminal globular domain, Accession No. PF00147) HMM having a score of at least 150-200, preferably at least 200-250, more preferably at least 250-300, and more preferably at least 300-350 or more is determinative of the presence of a C-terminal fibrinogen-like domain within a query protein. A search using the amino acid sequence of SEQ ID NO:2 was performed against the HMM database resulting in two hits to the fibrinogen_C HMM having scores of 162.3 and 69.8, respectively, resulting in the identification of a C-terminal fibrinogen-like domain in the amino acid sequence of SEQ ID NO:2. Accordingly, in one embodiment of the invention, a FDRG protein has a C-terminal fibrinogen-like domain at about amino acids 184-400 of SEQ ID NO:2. A search using the amino acid sequence of SEQ ID NO:12 was performed against the HMM database resulting in two hits to the fibrinogen_C HMM having scores of 160.9 and 66.3, respectively, resulting in the identification of a C-terminal fibrinogen-like domain in the amino acid sequence of SEQ ID NO:12. Accordingly, in another embodiment, a FDRG protein has a C-terminal fibrinogen-like domain domain at about amino acids 188-404 of SEQ ID NO:12. The C-terminal fibrinogen-like domains of human and mouse FDRG, as well as those of human angiopoietin, mouse angoipoietin-2, and ficolin are indicated by bold italics in FIG. 3.

In another embodiment, a FDRG family member is identified based on the presence of a “C-terminal fibrinogen-like domain” in the protein or corresponding nucleic acid molecule. As used herein, the term “C-terminal fibrinogen-like domain” includes a protein domain which is at least about 200-225 amino acid residues in length and has at least about 38-41% homology with human fibrinogen beta or gamma chain. In another embodiment, a “C-terminal fibrinogen-like domain” is at least about 100-300 amino acid residues in length, and preferably at least about 150-250 amino acid residues in length, and has at least about 25-60%, preferably at least about 30-50%, and more preferably at least about 35-45% homology with human fibrinogen beta or gamma chain. In yet another embodiment, a “C-terminal fibrinogen-like domain” refers to a protein domain which is at least about 100-300 amino acid residues in length, preferably at least about 150-250 amino acid residues in length, and more preferably at least about 200 amino acid residues in length, and has at least about 25-60%, preferably at least about 30-50%, more preferably at least about 35-45% homology, and even more preferably at least about 38-41% homology with amino acid residues 184-400 of SEQ ID NO:2. In a preferred embodiment, a C-terminal fibrinogen domain is involved in protein-protein interactions.

To identify the presence of a “C-terminal fibrinogen-like domain” in a FDRG family member, the amino acid sequence of the protein family member can be searched against a database of HMMs (e.g., the Pfam database, release 3.3) e.g., using the default parameters. For example, the search can be performed using the hmmsf program (family specific) and threshold score of 15 for determining a hit. hmmsf is available as part of the HMMER package of search programs (HMMER 2.1.1, December 1998) which is freely distributed by the Washington University school of medicine. In one embodiment, a hit to a fibrinogen_C (fibrinogen beta and gamma chains, C-terminal globular domain, Accession No. PF00147) HMM having a score of at least 150-200, preferably at least 200-250, more preferably at least 250-300, and more preferably at least 300-350 or more is determinative of the presence of a C-terminal fibrinogen-like domain within a query protein. A search using the amino acid sequence of SEQ ID NO:2 was performed against the HMM database resulting in two hits to the fibrinogen_C HMM having scores of 162.3 and 69.8, respectively, resulting in the identification of a C-terminal fibrinogen-like domain in the amino acid sequence of SEQ ID NO:2. Accordingly, in one embodiment of the invention, a FDRG protein has a C-terminal fibrinogen-like domain at about amino acids 184-400 of SEQ ID NO:2. A search using the amino acid sequence of SEQ ID NO:12 was performed against the HMM database resulting in two hits to the fibrinogen C HMM having scores of 160.9 and 66.3, respectively, resulting in the identification of a C-terminal fibrinogen-like domain in the amino acid sequence of SEQ ID NO:12. Accordingly, in another embodiment, a FDRG protein has a C-terminal fibrinogen-like domain domain at about amino acids 188-404 of SEQ ID NO:12. The C-terminal fibrinogen-like domains of human and mouse FDRG, as well as those of human angiopoietin, mouse angoipoietin-2, and ficolin are indicated by bold italics in FIG. 3.

A C-terminal fibrinogen-like domain can further contain at least about two, more preferably at least three, and even more preferably at least four cysteine residues which are conserved between FDRG molecules and members of the fibrinogen-like protein family (e.g., human angiopoietin 2, murine angiopoietin 2, human angiopoietin 1, and ficolin.) Preferably, the C-terminal fibrinogen-like domain of FDRG has cysteine residues which are located in the same or similar positions as cysteine residues in a fibrinogen-like protein family member. For example, when a FDRG protein of the invention is aligned with a FDRG family member or a fibrogen-like protein family member for purposes of comparison (see e.g., FIG. 3) preferred cysteine residues of the invention are those in which cysteine residues in the amino acid sequence of FDRG are located in the same or similar position as the cysteine residues in other FDRG family or fibrinogen-like family members. As an illustrative embodiment, FIG. 3 shows cysteine residues located in the same or similar positions of the human FDRG protein (corresponding to SEQ ID NO:2) and human angiopoietin-2 in the following locations: amino acid number 188 of human FDRG and amino acid number 284 of the human angiopoietin 2 protein; amino acid number 216 of human FDRG and amino acid number 313 of the human angiopoietin 2 protein; amino acid number 341 of human FDRG and amino acid number 437 of the human angiopoietin 2 protein; and amino acid number 354 of human FDRG and amino acid number 450 of the human angiopoietin 2 protein. Conserved cysteines are indicated by asterices in FIG. 3.

Another embodiment of the invention features FDRG molecules which contain an N-terminal unique domain. The term “N-terminal unique domain” as used herein, includes a protein domain of a FDRG protein family member which includes amino acid residues N-terminal to the N-terminus of a C-terminal fibrinogen-like domain in the amino acid sequence of the FDRG protein, e.g., a protein domain which includes amino acid residues from the N-terminal amino acid residue of the amino acid sequence of the protein to the N-terminus of the C-terminal fibrinogen-like domain. As used herein, an “N-terminal unique domain” includes a protein domain which is at least about 170-190 amino acid residues in length and has at least about 75-85% homology with the amino acid sequence of a second FDRG family member. In another embodiment, an N-terminal unique domain is at least about 150-210 amino acid residues in length, preferably at least about 160-200 amino acid residues in length, and has at least about 65-95%, preferably at least about 70-90% homology with the amino acid sequence of a second FDRG family member. As further defined herein, an N-terminal unique domain of a FDRG protein family member, however, is not sufficiently homologous to the amino acid sequence of a member of another protein family, such as an angiopoietin protein family. For example, an N-terminal unique domain of human FDRG (containing about amino acids 1-183 of SEQ ID NO:2 has at least about 80% homology to the N-terminal unique domain of murine FDRG (amino acid residues 1-187 of SEQ ID NO:12) but has no significant homology to the amino acid sequence of human angiopoietin 2.

As further defined herein, an N-terminal unique domain may further contain an “N-terminal signal sequence”. As used herein, a “signal sequence” refers to a peptide containing about 25 amino acids which occurs at the extreme N-terminal end of secretory and integral membrane proteins and which contains large numbers of hydrophobic amino acid residues. For example, a signal sequence contains at least about 15-35 amino acid residues, preferably about 18-32 amino acid residues, more preferably about 20-30 amino acid residues, and more preferably about 22-28 amino acid residues, and has at least about 40-70%, preferably about 50-65%, and more preferably about 55-60% hydrophobic amino acid residues (e.g., Alanine, Valine, Leucine, Isoleucine, Phenylalanine, or Proline). Such a “signal sequence”, also referred to in the art as a “signal peptide”, serves to direct a protein containing such a sequence to a lipid bilayer.

In a preferred embodiment, a FDRG protein contains both an N-terminal unique domain and a C-terminal fibrinogen-like domain. In another preferred embodiment, a FDRG N-terminal unique domain further contains a signal sequence. In one exemplary embodiment, a FDRG protein contains an N-terminal unique domain comprising amino acids 1-183 of SEQ ID NO:2 which further contains a signal sequence at amino acids 1-25 of SEQ ID NO:2. In yet another exemplary embodiment, a FDRG protein contains an N-terminal unique domain comprising amino acids 1-158 of SEQ ID NO:5. In another exemplary embodiment, a FDRG protein contains an N-terminal unique domain comprising amino acids 1-187 of SEQ ID NO:12 which further contains a signal sequence at amino acids 1-23 of SEQ ID NO:12. In yet another exemplary embodiment, a FDRG protein contains an N-terminal unique domain comprising amino acids 1-162 of SEQ ID NO:15.

In yet another embodiment, a FDRG protein encodes a mature FDRG protein. As used herein, the term “mature FDRG protein” refers to a FDRG protein from which the signal peptide has been cleaved. In an exemplary embodiment, a mature FDRG protein contains amino acid residues 1-381 of SEQ ID NO:5. In another exemplary embodiment, a mature FDRG protein contains amino acid residues 1-387 of SEQ ID NO:15. In a preferred embodiment, a FDRG protein is a mature FDRG protein which comprises a C-terminal fibrinogen-like domain.

Preferred FDRG molecules of the present invention have an amino acid sequence sufficiently homologous to the amino acid sequence of SEQ ID NO:2, SEQ ID NO:5, SEQ ID NO:12 or SEQ ID NO:15. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 60-65% homology, preferably 70-75% homology, more preferably 80-85%, and more preferably 90-95% homology across the amino acid sequences of the domains and contain at least one, preferably two, and more preferably three or four structural domains, are defined herein as sufficiently homologous. Furthermore, amino acid or nucleotide sequences that share at least 60-65%, preferably 70-75%, more preferably 80-85%, and even more preferably 90-95% homology and share a common functional activity are defined herein as sufficiently homologous.

As used interchangeably herein an “FDRG activity”, “biological activity of FDRG” or “functional activity of FDRG”, refers to an activity exerted by a FDRG protein, polypeptide or nucleic acid molecule on a FDRG responsive cell as determined in vivo, or in vitro, according to standard techniques. In one embodiment, a FDRG activity is a direct activity, such as an association with a cell-surface protein (e.g., a FDRG receptor or FDRG binding protein). In another embodiment, a FDRG activity is an indirect activity, such as the modulation of the activity of a second protein (e.g. a cellular signaling molecule) mediated by interaction of the FDRG protein with a cell surface protein. In a preferred embodiment, a FDRG activity is at least one or more of the following activities: (i) interaction of a FDRG protein in the extracellular milieu with a non-FDRG protein molecule (e.g., a FDRG receptor) on the surface of the same cell which secreted the FDRG protein molecule; (ii) interaction of a FDRG protein in the extracellular milieu with a non-FDRG protein molecule (e.g., a FDRG receptor) on the surface of a different cell from that which secreted the FDRG protein molecule; (iii) complex formation between a FDRG protein and a FDRG receptor; (iv) complex formation between a FDRG protein and non-FDRG receptor; (v) modulation of signal transduction via a cell surface protein (e.g., a FDRG receptor); (vi) modulation of the expression of adipogenic genes; (vii) modulation of a PPARγ function or modulation by a PPARγ function; (viii) modulation of a cellular response to a PPARγ ligand (e.g., thiazolidinediones) and (ix) interaction of a FDRG protein with a second protein in the extracellular milieu (e.g., a FDRG binding protein).

In yet another preferred embodiment, a FDRG activity is at least one or more of the following activities: (i) activation of a FDRG-dependent signal transduction pathway; (ii) modulation of angiogenesis; (iii) modulation of a hematopoiesis; (iv) modulation of the proliferation, development or differentiation of a FDRG-expressing cell or FDRG-receptor expressing cell (e.g., mediation of growth and/or differentiation of adipocytes, for example, white adipocytes or brown adipocytes); (v) modulation of the proliferation, development or differentiation of a non-FDRG-expressing cell or FDRG-expressing cell; (vi) modulation of the homeostasis of a FDRG-expressing cell; (vii) modulation of insulin sensitivity and/or insulin responsiveness; (viii) modulation of insulin secretion; (ix) modulation of cell recruitment; (x) modulation of the homeostasis of a non-FDRG-expressing cell; and (xi) modulation of glucose metabolism.

The present invention is based, at least in part, on the discovery of a novel secreted protein (the human FDRG protein having the amino acid sequence of SEQ ID NO:2) which was isolated from an aortic endothelial cDNA library, is highly expressed in placenta and fat, and has homology to angiopoietin. The present invention is based further in part on the discovery of a murine FDRG protein having the amino acid sequence of SEQ ID NO:12 which was isolated in a screen for transcriptional targets of PPARγ, a nuclear hormone receptor that can act as an adipogenic transcription factor and a potent regulator of systemic insulin action.

PPARγ is a nuclear hormone receptor which exists in two isoforms (γ1 and γ2) formed by alternative splicing (Zhu et al. (1995) PNAS 92:7921-7925) and which appears to function as both a direct regulator of many fat-specific genes and also as a “master” regulator that can trigger the entire program of adipogenesis (Spiegelman and Flier (1996) Cell 87:377-389). PPARγ forms a heterodimer with RXRα and has been shown to bind directly to well characterized fat-specific enhancers from the adipocyte P2 (aP2: Tontonoz (1994) Genes Dev. 8:1224-1234) and phosphoenolpyruvate carboxykinase (PEPCK) genes (Tontonoz (1994) Mol. Cell. Biol. 15:351-357). The identification of FDRG as a target of PPARγ places it in an important biological context, particularly in light of the prominent role the latter molecule plays in adipocyte differentiation and insulin signaling.

Murine FDRG is higly expressed in brown and white fat and placenta and expression is acutely induced in cultured cells by PPARγ ligands such as thiazolidinediones (TZDs). Murine FDRG expression is also upregulated in tissues of rodents chronically treated with TZDs. Furthermore, FDRG mRNA levels are elevated in murine models of obesity including the ob/ob and db/db mice, and are regulated on an acute basis by nutrition.

Accordingly, in another preferred embodiment, a FDRG activity is at least one or more of the following activities: (1) maintenance of energy homeostasis (e.g., regulating the balance and/or imbalance between energy storage and energy expenditure, for example, increasing/decreasing energy expenditure); (2) regulation of adaptive thermogenesis (e.g., regulation of the biogenesis of mitochondria, regulation of the expression of mitocrondrial enzymes, regulation of expression of uncoupling proteins; (3) regulation of adiposity; (4) regulation of lipolysis and/or lipogenesis; (6) modulation of the efficiency of energy storage; (6) regulation of appetite; (7) regulation of vasculogenesis (i.e., blood vessel formation or vascular remodeling); (8) regulation of tumor angiogenesis; (9) regulation of wound healing; (10) expansion/reduction of tumor mass; and (11) expansion/reduction of fat mass.

In yet another preferred embodiment, a FDRG protein or polypeptide is a secreted intercellular signalling molecule or factor (e.g., a secreted homeostatic factor) which is capable of modulating or regulating or modulates or regulates glucose metabolism and/or insulin sensitivity, for example, in tissues such as muscle and/or liver. In yet another embodiment, a FDRG protein or polypeptide is a secreted intercellular signalling molecule or factor which is capable of modulating or regulating or modulates or regulates (e.g., induces) angiogenesis and/or adipogenesis (e.g., fat development and/or differentiation.)

Moreover, in another embodiment of the invention, a FDRG molecule or preferably, a FDRG or FDRG receptor modulator, is useful for regulating, preventing and/or treating at least one or more of the following diseases or disorders: (1) obesity (e.g., excessive storage of energy as fat or a chronic imbalance between energy intake and expenditure); (2) diseases or disorders that accompany obesity (e.g., cardiovasculalar disorders); (3) diabetes (e.g., Type I or insulin dependent diabetes mellitus, Type II or non-insulin dependent diabetes mellitus, and/or insulin-resistent diabetes, both Type I and Type II); (4) disorders of energy homeostasis; (5) metabolic abnormalities typical of obesity (e.g., hyperinsulinemia); (6) insulin resistance; (7) disorders associated with lipid metabolism (e.g., cachexia); (8) atherosclerosis; and (9) disorders associated with abnormal vasculogenesis (e.g., cancers including, but not limited to, cancers of the epithelia (e.g., carcinomas of the pancreas, stomach, liver, secretory glands (e.g., adenocarcinoma), bladder, lung, breast, skin (e.g., fibromatosis or malignant melanoma), reproductive tract including prostate gland, ovary, cervix and uterus); cancers of the hematopoietic and immune system (e.g., leukemias and lymphomas); cancers of the central nervous, brain system and eye (e.g., gliomas, neuroblastoma and retinoblastoma); and cancers of connective tissues, bone, muscles and vasculature (e.g., hemangiomas and sarcomas)). In a preferred embodiment, a FDRG molecule, FDRG modulator or FDRG receptor modulator is useful for regulating, preventing and/or treating Type II diabetes mellitus and or obesity, insulin resistance, hyperinsulinemia and/or hypergyycemia associated with Type II diabetes mellitus. In another embodiment, a FDRG molecule or preferably, a FDRG or FDRG receptor modulator is an antidiabetic agent or an insulin sensitizing agent. In yet another embodiment, a FDRG molecule or preferably, a FDRG or FDRG receptor modulator is an angiogenic factor.

Accordingly, another embodiment of the invention features isolated FDRG proteins and polypeptides having a FDRG activity. Preferred FDRG proteins have a C-terminal fibrinogen-like domain and a FDRG activity. In another embodiment, the FDRG protein has an N-terminal unique domain, a C-terminal fibrinogen-like domain, and a FDRG activity. In another preferred embodiment, the FDRG protein has at least at least a C-terminal fibrinogen-like domain, an N-terminal unique domain, a FDRG activity, and an amino acid sequence sufficiently homologous to an amino acid sequence of SEQ ID NO:2, SEQ ID NO:5, SEQ ID NO:12 or SEQ ID NO:15.

In a particularly preferred embodiment, the FDRG protein and nucleic acid molecules of the present invention are human FDRG molecules. A human FDRG cDNA molecule was obtained from a human aortic endothelial cDNA library as described in Example 1. The nucleotide sequences of the isolated human FDRG cDNA and the predicted amino acid sequence of the human FDRG protein are shown in FIG. 1 and in SEQ ID NOs:1 and 2, respectively. In addition, the nucleotide sequence corresponding to the coding region of the human FDRG cDNA is represented as SEQ ID NO:3.

The human FDRG I cDNA set forth in SEQ ID NO:1, is approximately 1894 nucleotides in length and encodes a protein which is approximately 406 amino acid residues in length (SEQ ID NO:2). The human FDRG protein contains a signal sequence, an N-terminal unique domain, and a C-terminal fibrinogen-like domain, as defined herein. A FDRG N-terminal unique domain can be found at least, for example, from about amino acids 1-183 of SEQ ID NO:2 and, for example, from about amino acids 1-158 of SEQ ID NO:5. A FDRG C-terminal fibrinogen-like domain can be found at least, for example, from about amino acids 184-400 of SEQ ID NO:2 and, for example, from about amino acids 159-374 of SEQ ID NO:5. A signal sequence can be found at least, for example, from about amino acids 1-23 of SEQ ID NO:2. The prediction of such a signal peptide can be made, for example, utilizing the computer algorithm SIGNALP (Henrik, et al. (1997) Protein Engineering 10: 1-6).

In another particularly preferred embodiment, the FDRG protein and nucleic acid molecules of the present invention are murine FDRG molecules. A murine FDRG cDNA molecule was obtained from a screen designed to delect nucleic acids regulated by PPARγ, a fat-specific transcription factor, as described in Example 4. The nucleotide sequences of the isolated murine FDRG cDNA and the predicted amino acid sequence of the murine FDRG protein are shown in FIG. 4 and in SEQ ID NOs:11 and 12, respectively. In addition, the nucleotide sequence corresponding to the coding region of the murine FDRG cDNA is represented as SEQ ID NO:13.

The murine FDRG I cDNA set forth in SEQ ID NO:11, is approximately 1893 nucleotides in length and encodes a protein which is approximately 410 amino acid residues in length (SEQ ID NO:12). The murine FDRG protein contains a signal sequence, an N-terminal unique domain, and a C-terminal fibrinogen-like domain, as defined herein. A FDRG N-terminal unique domain can be found at least, for example, from about amino acids 1-187 of SEQ ID NO:12 and, for example, from about amino acids 1-162 of SEQ ID NO:15. A FDRG C-terminal fibrinogen-like domain can be found at least, for example, from about amino acids 188-404 of SEQ ID NO:12 and, for example, from about amino acids 163-379 of SEQ ID NO:15. A signal sequence can be found at least, for example, from about amino acids 1-23 of SEQ ID NO:12. The prediction of such a signal peptide can be made, for example, utilizing the computer algorithm SIGNALP (Henrik, et al. (1997) Protein Engineering 10:1-6).

FDRG mRNA expression was particularly high in placenta and fat as determined by norther blot analysis of various human and mouse tissues (See Examples 2 and 8).

An alignment of the amino acid sequences of human FDRG, murine FDRG, human angiopoietin 2 (Genbank™ Accession No. AF004327), murine angiopoietin 2 (Genbank™ Accession No. AF004326), and ficolin (Genbank™ Accession No. L112345) is shown in FIG. 3. (The alignment was generated using MegAlign™ sequence alignment software. The initial pairwise alignment step was performed using a Lipman Pearson algorithm with a K-tuple of 1, a GAP penalty of 3, a window of 5, and diagonals saved set to 5. The multiple alignment step was performed using the Clustal algorithm with a PAM 250 residue weight Table, a GAP penalty of 10, and a GAP length penalty of 10.)

Various aspects of the invention are described in further detail in the following subsections:

I. Isolated Nucleic Acid Molecules

One aspect of the invention pertains to isolated nucleic acid molecules that encode FDRG proteins or biologically active portions thereof, as well as nucleic acid fragments sufficient for use as hybridization probes to identify FDRG-encoding nucleic acids (e.g., FDRG mRNA) and fragments for use as PCR primers for the amplification or mutation of FDRG nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated FDRG nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a portion thereof, can be isolated using standard molecular biology techniques and the sequence information provided herein. Using all or portion of the nucleic acid sequence of SEQ ID NO:1, SEQ ID NO:3, SEQ ID NO:4, SEQ IDNO:11, SEQ ID NO:13, SEQ ID NO:14, or the nucleotide sequence of the DNA insert of the plasmid deposited with ATCC as Accession Number 98632, as a hybridization probe, FDRG nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632, can be isolated by the polymerase chain reaction (PCR) using synthetic oligonucleotide primers designed based upon the sequence of SEQ ID NO:1, SEQ ID NO:3, SEQ ID NO:4, or the nucleotide sequence of the DNA insert of the plasmid deposited with ATCC as Accession Number 98632.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to FDRG nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to a human FDRG cDNA. This cDNA comprises sequences encoding a human FDRG protein (i.e., “the coding region”, from nucleotides 166-1386), as well as 5′ untranslated sequences (nucleotides 1 to 165) and 3′ untranslated sequences (nucleotides 1387 to 1894). Alternatively, the nucleic acid molecule can comprise only the coding region of SEQ ID NO:1 (e.g., nucleotides 166 to 1386).

In another preferred embodiment, an isolated nucleic acid molecule of the invention comprises the nucleotide sequence shown in SEQ ID NO:3. The sequence of SEQ ID NO:3 corresponds to the human FDRG cDNA. This cDNA comprises sequences encoding the human FDRG protein (i.e., “the coding region”, from nucleotides 1 to 1218 of SEQ ID NO:3).

In another preferred embodiment, an isolated nucleic acid molecule of the invention comprises the nucleotide sequence shown in SEQ ID NO:4. The sequence of SEQ ID NO:4 corresponds to human FDRG cDNA. This cDNA comprises sequences encoding the mature FDRG protein (i.e., from nucleotides 241-1386 of SEQ ID NO:1 after the signal sequence has been cleaved).

In yet another preferred embodiment, an isolated nucleic acid molecule of the invention comprises the nucleotide sequence of the DNA insert of the plasmid deposited with ATCC as Accession Number 98632. A plasmid containing the full length nucleotide sequence encoding FDRG was deposited with the American Type Culture Collection (ATCC), Rockville, Md., on Jan. 14, 1997 and assigned Accession Number 98632.

In yet another preferred embodiment, an isolated nucleic acid molecule of the invention comprises the nucleotide sequence shown in SEQ ID NO:13. The sequence of SEQ ID NO:13 corresponds to a murine FDRG cDNA. This cDNA comprises sequences encoding a murine FDRG protein (i.e., “the coding region”, from nucleotides 149-1378), as well as 5′ untranslated sequences (nucleotides 1 to 148) and 3′ untranslated sequences (nucleotides 1379 to 1893). Alternatively, the nucleic acid molecule can comprise only the coding region of SEQ ID NO:13 (e.g., nucleotides 149 to 1378).

In another preferred embodiment, an isolated nucleic acid molecule of the invention comprises the nucleotide sequence shown in SEQ ID NO:13. The sequence of SEQ ID NO:13 corresponds to the murine FDRG cDNA. This cDNA comprises sequences encoding the murine FDRG protein (i.e., “the coding region”, from nucleotides 1 to 1230 of SEQ ID NO:13).

In another preferred embodiment, an isolated nucleic acid molecule of the invention comprises the nucleotide sequence shown in SEQ ID NO:14. The sequence of SEQ ID NO:14 corresponds to murine FDRG cDNA. This cDNA comprises sequences encoding the mature FDRG protein (i.e., from nucleotides 710-1380 of SEQ ID NO:11 after the signal sequence has been cleaved).

In another preferred embodiment, an isolated nucleic acid molecule of the invention comprises a nucleic acid molecule which is a complement of the nucleotide sequence shown in SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a portion of any of these nucleotide sequences. A nucleic acid molecule which is complementary to the nucleotide sequence shown in SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632 is one which is sufficiently complementary to the nucleotide sequence shown in SEQ ID NO:1, SEQ ID NO:3, SEQ ID NO:4, SEQ ID NO:1, SEQ ID NO:13, SEQ ID NO:14, or the nucleotide sequence of the DNA insert of the plasmid deposited with ATCC as Accession Number 98632 such that it can hybridize to the nucleotide sequence shown in SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632, thereby forming a stable duplex.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95% or more homologous to the nucleotide sequence shown in SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a portion of any of these nucleotide sequences. In still another preferred embodiment, an isolated nucleic acid molecule of the present invention comprises a nucleotide sequence which is at least about 61%, 62%, 63%, 64%, 65%, 66%, 67%, 68%, or 69% homologous, preferably at least about 71%, 72%, 73%, 74%, 75%, 76%, 77%, 78%, or 79% homologous, more preferably at least about 81%, 82%, 83%, 84%, 85%, 86%, 87%, 88%, or 89% homologous, and even more preferably at least about 91%, 92%, 93%, 94%, 95%, 96%, 97%, 98%, or 99% or more homologous to the nucleotide sequence shown in SEQ ID NO:1, SEQ ID NO:3, SEQ ID NO:4, SEQ ID NO:11, SEQ ID NO:13, SEQ ID NO:14, the nucleotide sequence of the DNA insert of the plasmid deposited with ATCC as Accession Number 98632, or a portion of any of these nucleotide sequences.

Moreover, the nucleic acid molecule of the invention can comprise only a portion of the nucleic acid sequence of SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632, for example a fragment which can be used as a probe or primer or a fragment encoding a biologically active portion of FDRG. The nucleotide sequence determined from the cloning of the human and murine FDRG genes allows for the generation of probes and primers designed for use in identifying and/or cloning FDRG homologues in other cell types, e.g., from other tissues, as well as FDRG homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632 sense, or an anti-sense sequence of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632, or of a naturally occurring mutant of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632.

Probes based on the human FDRG nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a FDRG protein, such as by measuring a level of a FDRG-encoding nucleic acid in a sample of cells from a subject e.g., detecting FDRG mRNA levels or determining whether a genomic FDRG gene has been mutated or deleted.

A nucleic acid fragment encoding a “biologically active portion of FDRG” can be prepared by isolating a portion of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632 which encodes a polypeptide having a FDRG biological activity (the biological activities of the FDRG proteins have previously been described), expressing the encoded portion of FDRG protein (e.g., by recombinant expression in vitro) and assessing the activity of the encoded portion of FDRG.

The invention further encompasses nucleic acid molecules that differ from the nucleotide sequence shown in SEQ ID NO:1, SEQ ID NO:3, SEQ ID NO:4 and from the nucleotide sequences of the DNA insert of the plasmid deposited with ATCC as Accession Number 98632, or portions thereof, due to degeneracy of the genetic code and thus encode the same FDRG protein as that encoded by the nucleotide sequence shown in SEQ ID NO:1, SEQ ID NO:3 or SEQ ID NO:4 or by the nucleotide sequence of the DNA insert of the plasmid deposited with ATCC as Accession Number 98632. In another embodiment, an isolated nucleic acid molecule of the invention has a nucleotide sequence encoding a protein having an amino acid sequence shown in SEQ ID NO:2, SEQ ID NO:5, SEQ ID NO:12 or SEQ ID NO:15.

In addition to the human FDRG nucleotide sequences shown in SEQ ID NO:1, SEQ ID NO:3 and SEQ ID NO:4, respectively, it will be appreciated by those skilled in the art that DNA sequence polymorphisms that lead to changes in the amino acid sequences of FDRG may exist within a population (e.g., the human population). Such genetic polymorphism in the FDRG gene may exist among individuals within a population due to natural allelic variation. As used herein, the terms “gene” and “recombinant gene” refer to nucleic acid molecules comprising an open reading frame encoding a FDRG protein, preferably a mammalian FDRG protein. Such natural allelic variations can typically result in 1-5% variance in the nucleotide sequence of the FDRG gene. Any and all such nucleotide variations and resulting amino acid polymorphisms in FDRG that are the result of natural allelic variation and that do not alter the functional activity of FDRG are intended to be within the scope of the invention.

Moreover, nucleic acid molecules encoding FDRG proteins from other species, and thus which have a nucleotide sequence which differs from the human sequences of SEQ ID NO:1, SEQ ID NO:3, SEQ ID NO:4, SEQ ID NO:11, SEQ ID NO:13, SEQ ID NO:14, or the nucleotide sequence of the DNA insert of the plasmid deposited with ATCC as Accession Number 98632 are intended to be within the scope of the invention. Nucleic acid molecules corresponding to natural allelic variants and homologues of the FDRG cDNA of the invention can be isolated based on their homology to the human FDRG nucleic acids disclosed herein using the human cDNA, or portions thereof, as a hybridization probe according to standard hybridization techniques under stringent hybridization conditions. For example, a viral FDRG cDNA can be isolated based on its homology to human FDRG.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98613. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1, SEQ ID NO:3 or SEQ ID NO:4 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the FDRG sequence that may exist in the population, the skilled artisan will further appreciate that changes can be introduced by mutation into the nucleotide sequence of SEQ ID NO:1, SEQ ID NO:3, SEQ ID NO:4, or the DNA insert of the plasmid deposited with ATCC as Accession Number 98632, thereby leading to changes in the amino acid sequence of the encoded FDRG protein, without altering the functional ability of the FDRG protein. For example, nucleotide substitutions leading to amino acid substitutions at “non-essential” amino acid residues can be made in the sequence of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632. A “non-essential” amino acid residue is a residue that can be altered from the wild-type sequence of FDRG (e.g., the sequence of SEQ ID NO:2, SEQ ID NO:5, SEQ ID NO:12 or SEQ ID NO:15) without altering the biological activity, whereas an “essential” amino acid residue is required for biological activity. For example, amino acid residues that are conserved among the FDRG proteins of the present invention, are predicted to be particularly unamenable to alteration. Furthermore, amino acid residues that are conserved between FDRG protein and other proteins having fibrinogen-like domains are not likely to be amenable to alteration.

Accordingly, another aspect of the invention pertains to nucleic acid molecules encoding FDRG proteins that contain changes in amino acid residues that are not essential for activity. Such FDRG proteins differ in amino acid sequence from SEQ ID NO:2, SEQ ID NO:5, SEQ ID NO:12 or SEQ ID NO:15 yet retain biological activity. In one embodiment, the isolated nucleic acid molecule comprises a nucleotide sequence encoding a protein, wherein the protein comprises an amino acid sequence at least about 60-65% homologous to the amino acid sequence of SEQ ID NO:2. Preferably, the protein encoded by the nucleic acid molecule is at least about 70-75% homologous to SEQ ID NO:2, SEQ ID NO:5, SEQ ID NO:12, or SEQ ID NO:15, more preferably at least about 80-85% homologous to SEQ ID NO:2, SEQ ID NO:5, SEQ ID NO:12, or SEQ ID NO:15, and even more preferably at least about 90-95% homologous to SEQ ID NO:2, SEQ ID NO:5, SEQ ID NO:12, or SEQ ID NO:15.

In an alternative embodiment, the isolated nucleic acid molecule comprises a nucleotide sequence encoding a protein, wherein the protein comprises an amino acid sequence at least about 61%, 62%, 63%, 64%, 65%, 66%, 67%, 68%, or 69% homologous, preferably at least about 71%, 72%, 73%, 74%, 75%, 76%, 77%, 78%, or 79% homologous, more preferably at least about 81%, 82%, 83%, 84%, 85%, 86%, 87%, 88%, or 89% homologous, and even more preferably at least about 91%, 92%, 93%, 94%, 95%, 96%, 97%, 98%, or 99% or more homologous to SEQ ID NO:2, SEQ ID NO:5, SEQ ID NO:12, or SEQ ID NO:15.

An isolated nucleic acid molecule encoding a FDRG protein homologous to the protein of SEQ ID NO:2, SEQ ID NO:5, SEQ ID NO:12 or SEQ ID NO:15 can be created by introducing one or more nucleotide substitutions, additions or deletions into the nucleotide sequence of SEQ ID NO:1, SEQ ID NO:3 or SEQ ID NO:4, or into the nucleotide sequence of the DNA insert of the plasmid deposited with ATCC as Accession Number 98632, such that one or more amino acid substitutions, additions or deletions are introduced into the encoded protein. Mutations can be introduced into SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in FDRG is preferably replaced with another amino acid residue from the same side chain family. Alternatively, in another embodiment, mutations can be introduced randomly along all or part of a FDRG coding sequence, such as by saturation mutagenesis, and the resultant mutants can be screened for FDRG biological activity to identify mutants that retain activity. Following mutagenesis of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632, the encoded protein can be expressed recombinantly and the activity of the protein can be determined.

In a preferred embodiment, a mutant FDRG protein can be assayed for (1) activation of a FDRG-dependent signal transduction pathway; (2) modulation of secretion of a non-IL-17 cytokine; (3) modulation of secretion of IL-17; (4) modulation of surface expression of a cellular adhesion molecule; (5) modulation of a proinflammatory cytokine; (6) modulation of a hematopoietic cytokine; (7) modulation of the development or differentiation of a FDRG-expressing cell; (8) modulation of the development or differentiation of a non-FDRG-expressing cell; (9) modulation of the homeostasis of a FDRG-expressing cell; (10) modulation of the homeostasis of a non-FDRG-expressing cell; (11) modulation of adipocyte development and physiology; and (12) modulation of insulin signaling.

In addition to the nucleic acid molecules encoding FDRG proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire FDRG coding strand, or to only a portion thereof. In one embodiment, an antisense nucleic acid molecule is antisense to a “coding region” of the coding strand of a nucleotide sequence encoding FDRG. The term “coding region” refers to the region of the nucleotide sequence comprising codons which are translated into amino acid residues (e.g., the coding region of human FDRG corresponds to SEQ ID NO:3 and the coding region of murine FDRG corresponds to SEQ ID NO:13). In another embodiment, the antisense nucleic acid molecule is antisense to a “noncoding region” of the coding strand of a nucleotide sequence encoding FDRG. The term “noncoding region” refers to 5′ and 3′ sequences which flank the coding region that are not translated into amino acids (i.e., also referred to as 5′ and 3′ untranslated regions).

Given the coding strand sequences encoding FDRG disclosed herein (e.g., SEQ ID NO:3), antisense nucleic acids of the invention can be designed according to the rules of Watson and Crick base pairing. The antisense nucleic acid molecule can be complementary to the entire coding region of FDRG mRNA, but more preferably is an oligonucleotide which is antisense to only a portion of the coding or noncoding region of FDRG mRNA. For example, the antisense oligonucleotide can be complementary to the region surrounding the translation start site of FDRG mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, I-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a FDRG protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave FDRG mRNA transcripts to thereby inhibit translation of FDRG mRNA. A ribozyme having specificity for a FDRG-encoding nucleic acid can be designed based upon the nucleotide sequence of a FDRG cDNA disclosed herein (i.e., SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632). For example, a derivative of a Tetrahymena L-19 IVS RNA can be constructed in which the nucleotide sequence of the active site is complementary to the nucleotide sequence to be cleaved in a FDRG-encoding mRNA. See, e.g., Cech et al. U.S. Pat. No. 4,987,071; and Cech et al. U.S. Pat. No. 5,116,742. Alternatively, FDRG mRNA can be used to select a catalytic RNA having a specific ribonuclease activity from a pool of RNA molecules. See, e.g., Bartel, D. and Szostak, J. W. (1993) Science 261:1411-1418.

Alternatively, FDRG gene expression can be inhibited by targeting nucleotide sequences complementary to the regulatory region of the FDRG (e.g., the FDRG promoter and/or enhancers) to form triple helical structures that prevent transcription of the FDRG gene in target cells. See generally, Helene, C. (1991) Anticancer Drug Des. 6(6):569-84; Helene, C. et al. (1992) Ann. N. Y Acad. Sci. 660:27-36; and Maher, L. J. (1992) Bioassays 14(12):807-15.

In preferred embodiments, the nucleic acids of FDRG can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of FDRG can be used therapeutic and diagnostic applications. For example, PNAs can be used as antisense or antigene agents for sequence-specific modulation of gene expression by, e.g., inducing trascription or translation arrest or inhibiting replication. PNAs of FDRG can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) supra); or as probes or primers for DNA sequence and hybridization (Hyrup B. et al. (1996) supra; Perry-O'Keefe supra).

In another embodiment, PNAs of FDRG can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of FDRG can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Research 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988). In addition, oligonucleotides can be modified with hybridization-triggered cleavage agents (See, e.g., Krol et al., 1988, Bio Techniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

In addition to the isolated nucleic acid molecules described above, the present invention also features isolated “genes” corresponding to the nucleic acid molecules of SEQ ID NO:1, SEQ ID NO:3, SEQ ID NO:4, SEQ ID NO:11, SEQ ID NO:13 or SEQ ID NO:14. As defined herein, the term “isolated gene” includes a nucleic acid molecule comprising introns and exons which is separated from genomic DNA sequences which naturally flank the gene (i.e., sequences located at the 5′ and 3′ ends of the naturally-occurring gene) in the genomic DNA of the organism from which the isolated gene is derived. An isolated gene corresponding, for example, to the isolated nucleic acid molecules of SEQ ID NO:11, SEQ ID NO:13 or SEQ ID NO:14 is set forth as SEQ ID NO:27 and described in Example 7 herein. As used herein, an isolated gene “corresponding to” the isolated nucleic acid molecules of SEQ ID NO:11, SEQ ID NO:13 or SEQ ID NO:14 includes an isolated gene having a nucleotide sequence which includes fragments of contiguous nucleotides of the nucleic acid sequence of the isolated nucleic acid molecules of SEQ ID NO:11, SEQ ID NO:13 or SEQ ID NO:14 (e.g., cDNA sequence corresponding to exons in the isolated gene), such fragments being interspersed with nucleotide sequence not set forth in SEQ ID NO:11, SEQ ID NO:13 or SEQ ID NO:14 (e.g., cDNA sequence corresponding to introns in the isolated gene). In a preferred embodiment, the present invention features an isolated gene corresponding to SEQ ID NO:11, SEQ ID NO:13 or SEQ ID NO:14. In another embodiment, the invention features an isolated gene comprising the nucleotide sequence of SEQ ID NO:27. Moreover, using the methods described in particular, in Example 7 herein, one can use all or a portion of the isolated nucleic acid molecules set forth as SEQ ID NO:1, SEQ ID NO:3 or SEQ ID NO:4 to isolate a gene corresponding to the isolated nucleic acid molecule of SEQ ID NO:1, SEQ ID NO:3 or SEQ ID NO:4. Accordingly, in another embodiment, the present invention features an isolated gene corresponding to SEQ ID NO:1, SEQ ID NO:3 or SEQ ID NO:4.

II. Isolated FDRG Proteins and Anti-FDRG Antibodies

One aspect of the invention pertains to isolated FDRG proteins, and biologically active portions thereof, as well as polypeptide fragments suitable for use as immunogens to raise anti-FDRG antibodies. In one embodiment, native FDRG proteins can be isolated from cells or tissue sources by an appropriate purification scheme using standard protein purification techniques. In another embodiment, FDRG proteins are produced by recombinant DNA techniques. Alternative to recombinant expression, a FDRG protein or polypeptide can be synthesized chemically using standard peptide synthesis techniques.

An “isolated” or “purified” protein or biologically active portion thereof is substantially free of cellular material or other contaminating proteins from the cell or tissue source from which the FDRG protein is derived, or substantially free from chemical precursors or other chemicals when chemically synthesized. The language “substantially free of cellular material” includes preparations of FDRG protein in which the protein is separated from cellular components of the cells from which it is isolated or recombinantly produced. In one embodiment, the language “substantially free of cellular material” includes preparations of FDRG protein having less than about 30% (by dry weight) of non-FDRG protein (also referred to herein as a “contaminating protein”), more preferably less than about 20% of non-FDRG protein, still more preferably less than about 10% of non-FDRG protein, and most preferably less than about 5% non-FDRG protein. When the FDRG protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of FDRG protein in which the protein is separated from chemical precursors or other chemicals which are involved in the synthesis of the protein. In one embodiment, the language “substantially free of chemical precursors or other chemicals” includes preparations of FDRG protein having less than about 30% (by dry weight) of chemical precursors or non-FDRG chemicals, more preferably less than about 20% chemical precursors or non-FDRG chemicals, still more preferably less than about 10% chemical precursors or non-FDRG chemicals, and most preferably less than about 5% chemical precursors or non-FDRG chemicals.

Biologically active portions of a FDRG protein include peptides comprising amino acid sequences sufficiently homologous to or derived from the amino acid sequence of the FDRG protein, e.g., the amino acid sequence shown in SEQ ID NO:2, SEQ ID NO:5, SEQ ID NO:12 or SEQ ID NO:15, which include less amino acids than the full length FDRG proteins, and exhibit at least one activity of a FDRG protein. Typically, biologically active portions comprise a domain or motif with at least one activity of the FDRG protein. A biologically active portion of a FDRG protein can be a polypeptide which is, for example, 10, 25, 50, 100 or more amino acids in length.

In one embodiment, a biologically active portion of a FDRG protein comprises at least a C-terminal fibrinogen-like domain. In yet another embodiment, a biologically active portion of a FDRG protein comprises at least a signal sequence. In an alternative embodiment, a biologically active portion of a FDRG protein comprises a FDRG amino acid sequence lacking a signal sequence.

It is to be understood that a preferred biologically active portion of a FDRG protein of the present invention may contain at least one of the above-identified structural domains. A more preferred biologically active portion of a FDRG protein may contain at least two of the above-identified structural domains. An even more preferred biologically active portion of a FDRG protein may contain at least three or more of the above-identified structural domains.

Moreover, other biologically active portions, in which other regions of the protein are deleted, can be prepared by recombinant techniques and evaluated for one or more of the functional activities of a native FDRG protein.

In a preferred embodiment, the FDRG protein has an amino acid sequence shown in SEQ ID NO:2, SEQ ID NO:5, SEQ ID NO:12 or SEQ ID NO:15. In other embodiments, the FDRG protein is substantially homologous to SEQ ID NO:2, SEQ ID NO:5, SEQ ID NO:12 or SEQ ID NO:15 and retains the functional activity of the protein of SEQ ID NO:2, SEQ ID NO:5, SEQ ID NO:12 or SEQ ID NO:15 yet differs in amino acid sequence due to natural allelic variation or mutagenesis, as described in detail in subsection I above. Accordingly, in another embodiment, the FDRG protein is a protein which comprises an amino acid sequence at least about 60% homologous to the amino acid sequence of SEQ ID NO:2, SEQ ID NO:5, SEQ ID NO:12 or SEQ ID NO:15 and retains the functional activity of the FDRG proteins of SEQ ID NO:2, SEQ ID NO:5, SEQ ID NO:12 or SEQ ID NO:15, respectively. Preferably, the protein is at least about 70% homologous to SEQ ID NO:2, SEQ ID NO:5, SEQ ID NO:12 or SEQ ID NO:15, more preferably at least about 80% homologous to SEQ ID NO:2, SEQ ID NO:5, SEQ ID NO:12 or SEQ ID NO:15, even more preferably at least about 90% homologous to SEQ ID NO:2, SEQ ID NO:5, SEQ ID NO:12 or SEQ ID NO:15, and most preferably at least about 95% or more homologous to SEQ ID NO:2, SEQ ID NO:5, SEQ ID NO:12 or SEQ ID NO:15.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the FDRG amino acid sequence of SEQ ID NO:2, having 86 amino acid residues, at least 66, preferably at least 46, more preferably at least 26 are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=#of identical positions/total #of positions×100).

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to FDRG nucleic acid molecules of the invention. BLAST protein searches can be performed with the XBLAST program, score=50, wordlength=3 to obtain amino acid sequences homologous to TAP-1 protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used. Alternatively, a PAM 250 residue weight Table, a GAP penalty of 10, and a GAP length penalty of 10 can be used. Another preferred, non-limiting example of a mathematical algorithim utilized for the comparison of sequences is the algorithm of Wilbur-Lipmann which is part of MegAlign™ sequence alignment software. When utilizing the Wilbur-Lipmann algorithm, a K-tuple of 1, a GAP penalty of 3, a window of 5, and diagonals saved set to=5 can be used. Multiple alignment can be performed using the Clustal algorithm.

The invention also provides FDRG chimeric or fusion proteins. As used herein, a FDRG “chimeric protein” or “fusion protein” comprises a FDRG polypeptide operatively linked to a non-FDRG polypeptide. A “FDRG polypeptide” refers to a polypeptide having an amino acid sequence corresponding to FDRG, whereas a “non-FDRG polypeptide” refers to a polypeptide having an amino acid sequence corresponding to a protein which is not substantially homologous to the FDRG protein, e.g., a protein which is different from the FDRG protein and which is derived from the same or a different organism. Within a FDRG fusion protein the FDRG polypeptide can correspond to all or a portion of a FDRG protein. In a preferred embodiment, a FDRG fusion protein comprises at least one biologically active portion of a FDRG protein. In another preferred embodiment, a FDRG fusion protein comprises at least two biologically active portions of a FDRG protein. In another preferred embodiment, a FDRG fusion protein comprises at least three biologically active portions of a FDRG protein. Within the fusion protein, the term “operatively linked” is intended to indicate that the FDRG polypeptide and the non-FDRG polypeptide are fused in-frame to each other. The non-FDRG polypeptide can be fused to the N-terminus or C-terminus of the FDRG polypeptide.

For example, in one embodiment, the fusion protein is a GST-FDRG fusion protein in which the FDRG sequences are fused to the C-terminus of the GST sequences. Such fusion proteins can facilitate the purification of recombinant FDRG.

In another embodiment, the fusion protein is a FDRG protein containing a heterologous signal sequence at its N-terminus. For example, the native FDRG signal sequence (i.e, about amino acids 1 to 25 of SEQ ID NO:2) can be removed and replaced with a signal sequence from another protein. In certain host cells (e.g., mammalian host cells), expression and/or secretion of FDRG can be increased through use of a heterologous signal sequence.

In yet another embodiment, the fusion protein is a FDRG-immunoglobulin fusion protein in which the FDRG sequences comprising primarily the FDRG extracellular domain are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon, D. J. et al. (1989) Nature 337:525-531 and Capon U.S. Pat. Nos. 5,116,964 and 5,428,130 [CD4-IgG1 constructs]; Linsley, P. S. et al. (1991) J. Exp. Med. 173:721-730 [a CD28-IgG1 construct and a B7-1-IgG1 construct]; and Linsley, P. S. et al. (1991) J. Exp. Med. 174:561-569 and U.S. Pat. No. 5,434,131[a CTLA4-IgG1]). Such fusion proteins have proven useful for modulating receptor-ligand interactions. Soluble derivatives of cell surface proteins of the tumor necrosis factor receptor (TNFR) superfamily proteins have been made consisting of an extracellular domain of the cell surface receptor fused to an immunoglobulin constant (Fc) region (see for example Moreland et al. (1997) N. Engl. J. Med. 337(3):141-147; van der Poll et al. (1997) Blood 89(10):3727-3734; and Ammann et al. (1997) J. Clin. Invest. 99(7):1699-1703.) Furthermore, fusion proteins have been made using the CH2 and CH3 domains of IgG fused downstream of murine IL-17 leader sequences and upstream of murine CTLA-8 sequences and upstream of HVS13 sequences (see for example Yao et al. (1995) Immunity 8:811-821.)

The FDRG-immunoglobulin fusion proteins of the invention can be incorporated into pharmaceutical compositions and administered to a subject to inhibit an interaction between a FDRG protein and a FDRG receptor on the surface of a cell, to thereby suppress FDRG-mediated cellular function in vivo. The FDRG-immunoglobulin fusion proteins can be used to affect the bioavailability of a FDRG protein. Inhibition of the FDRG protein/FDRG receptor interaction may be useful therapeutically, for example, in regulation of the cellular immune response, regulation of inflammation, regulation of hematopoiesis, regulation of adipogenesis, or regulation of angiogenesis. Moreover, the FDRG-immunoglobulin fusion proteins of the invention can be used as immunogens to produce anti-FDRG antibodies in a subject, to purify FDRG receptors and in screening assays to identify molecules which inhibit the interaction of a FDRG protein with a FDRG receptor.

Preferably, a FDRG chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A FDRG-encoding nucleic acid can be cloned into such an expression vector such that the fusion moiety is linked in-frame to the FDRG protein.

The present invention also pertains to variants of the FDRG proteins which function as either FDRG agonists (mimetics) or as FDRG antagonists. Variants of the FDRG protein can be generated by mutagenesis, e.g., discrete point mutation or truncation of the FDRG protein. An agonist of the FDRG protein can retain substantially the same, or a subset, of the biological activities of the naturally occurring form of the FDRG protein. An antagonist of the FDRG protein can inhibit one or more of the activities of the naturally occurring form of the FDRG protein by, for example, competitively binding to a FDRG receptor of FDRG-binding protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the FDRG proteins.

In one embodiment, variants of the FDRG protein which function as either FDRG agonists (mimetics) or as FDRG antagonists can be identified by screening combinatorial libraries of mutants, (e.g., truncation mutants) of the FDRG protein for FDRG protein agonist or antagonist activity. In one embodiment, a variegated library of FDRG variants is generated by combinatorial mutagenesis at the nucleic acid level and is encoded by a variegated gene library. A variegated library of FDRG variants can be produced by, for example, enzymatically ligating a mixture of synthetic oligonucleotides into gene sequences such that a degenerate set of potential FDRG sequences is expressible as individual polypeptides, or alternatively, as a set of larger fusion proteins (e.g., for phage display) containing the set of FDRG sequences therein. There are a variety of methods which can be used to produce libraries of potential FDRG variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential FDRG sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the FDRG protein coding sequence can be used to generate a variegated population of FDRG fragments for screening and subsequent selection of variants of a FDRG protein. In one embodiment, a library of coding sequence fragments can be generated by treating a double stranded PCR fragment of a FDRG coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the FDRG protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of FDRG proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify FDRG variants (Arkin and Yourvan (1992) PNAS 89:7811-7815; Delgrave et al. (1993) Protein Engineering 6(3):327-331).

In one embodiment, cell based assays can be exploited to analyze a variegated FDRG library. For example, a library of expression vectors can be transfected into a cell line which ordinarily secretes FDRG protein. Supernatants from the transfected cells are then contacted with FDRG-responsive cells and the effect of the mutation in FDRG can be detected, e.g., by measuring any of a number of FDRG-responsive cell responses. Plasmid DNA can then be recovered from the mutant FDRG-secreting cells which score for inhibition, or alternatively, potentiation of the FDRG-dependent response, and the individual clones further characterized.

An isolated FDRG protein, or a portion or fragment thereof, can be used as an immunogen to generate antibodies that bind FDRG using standard techniques for polyclonal and monoclonal antibody preparation. The full-length FDRG protein can be used or, alternatively, the invention provides antigenic peptide fragments of FDRG for use as immunogens. The antigenic peptide of FDRG comprises at least 8 amino acid residues of the amino acid sequence shown in SEQ ID NO:2, SEQ ID NO:5, SEQ ID NO:12 or SEQ ID NO:15 and encompasses an epitope of FDRG such that an antibody raised against the peptide forms a specific immune complex with FDRG. Preferably, the antigenic peptide comprises at least 10 amino acid residues, more preferably at least 15 amino acid residues, even more preferably at least 20 amino acid residues, and most preferably at least 30 amino acid residues.

A FDRG immunogen typically is used to prepare antibodies by immunizing a suitable subject, (e.g., rabbit, goat, mouse or other mammal) with the immunogen. An appropriate immunogenic preparation can contain, for example, recombinantly expressed FDRG protein or a chemically synthesized FDRG polypeptide. The preparation can further include an adjuvant, such as Freund's complete or incomplete adjuvant, or similar immunostimulatory agent. Immunization of a suitable subject with an immunogenic FDRG preparation induces a polyclonal anti-FDRG antibody response.

Accordingly, another aspect of the invention pertains to anti-FDRG antibodies. The term “antibody” as used herein refers to immunoglobulin molecules and immunologically active portions of immunoglobulin molecules, i.e., molecules that contain an antigen binding site which specifically binds (immunoreacts with) an antigen, such as FDRG. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind FDRG. The term “monoclonal antibody” or “monoclonal antibody composition”, as used herein, refers to a population of antibody molecules that contain only one species of an antigen binding site capable of immunoreacting with a particular epitope of FDRG. A monoclonal antibody composition thus typically displays a single binding affinity for a particular FDRG protein with which it immunoreacts.

Polyclonal anti-FDRG antibodies can be prepared as described above by immunizing a suitable subject with a FDRG immunogen. The anti-FDRG antibody titer in the immunized subject can be monitored over time by standard techniques, such as with an enzyme linked immunosorbent assay (ELISA) using immobilized FDRG. If desired, the antibody molecules directed against FDRG can be isolated from the mammal (e.g., from the blood) and further purified by well known techniques, such as protein A chromatography to obtain the IgG fraction. At an appropriate time after immunization, e.g., when the anti-FDRG antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) PNAS 76:2927-31; and Yeh et al. (1982) Int. J Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med, 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a FDRG immunogen as described above, and the culture supernatants of the resulting hybridoma cells are screened to identify a hybridoma producing a monoclonal antibody that binds FDRG.

Any of the many well known protocols used for fusing lymphocytes and immortalized cell lines can be applied for the purpose of generating an anti-FDRG monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind FDRG, e.g., using a standard ELISA assay.

Alternative to preparing monoclonal antibody-secreting hybridomas, a monoclonal anti-FDRG antibody can be identified and isolated by screening a recombinant combinatorial immunoglobulin library (e.g., an antibody phage display library) with FDRG to thereby isolate immunoglobulin library members that bind FDRG. Kits for generating and screening phage display libraries are commercially available (e.g., the Pharmacia Recombinant Phage Antibody System, Catalog No. 27-9400-01; and the Stratagene SurfZaP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-FDRG antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Bio Techniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-FDRG antibody (e.g., monoclonal antibody) can be used to isolate FDRG by standard techniques, such as affinity chromatography or immunoprecipitation. An anti-FDRG antibody can facilitate the purification of natural FDRG from cells and of recombinantly produced FDRG expressed in host cells. Moreover, an anti-FDRG antibody can be used to detect FDRG protein (e.g., in a cellular lysate or cell supernatant) in order to evaluate the abundance and pattern of expression of the FDRG protein. Anti-FDRG antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding FDRG (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., FDRG proteins, mutant forms of FDRG, fusion proteins, etc.).

The recombinant expression vectors of the invention can be designed for expression of FDRG in prokaryotic or eukaryotic cells. For example, FDRG can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promotors directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in FDRG activity assays, in FDRG ligand binding (e.g., direct assays or competitive assays described in detail below), to generate antibodies specific for FDRG proteins, as examples. In a preferred embodiment, a FDRG fusion expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the FDRG expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, Carlsbad, Calif.).

Alternatively, FDRG can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to FDRG mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, FDRG protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding FDRG or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) FDRG protein. Accordingly, the invention further provides methods for producing FDRG protein using the host cells of the invention. In one embodiment, the method comprises culturing the host cell of invention (into which a recombinant expression vector encoding FDRG has been introduced) in a suitable medium such that FDRG protein is produced. In another embodiment, the method further comprises isolating FDRG from the medium or the host cell.

The host cells of the invention can also be used to produce nonhuman transgenic animals. For example, in one embodiment, a host cell of the invention is a fertilized oocyte or an embryonic stem cell into which FDRG-coding sequences have been introduced. Such host cells can then be used to create non-human transgenic animals in which exogenous FDRG sequences have been introduced into their genome or homologous recombinant animals in which endogenous FDRG sequences have been altered. Such animals are useful for studying the function and/or activity of FDRG and for identifying and/or evaluating modulators of FDRG activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous FDRG gene has been altered by homologous recombination between the endogenous gene and an exogenous DNA molecule introduced into a cell of the animal, e.g., an embryonic cell of the animal, prior to development of the animal.

A transgenic animal of the invention can be created by introducing FDRG-encoding nucleic acid into the male pronuclei of a fertilized oocyte, e.g., by microinjection, retroviral infection, and allowing the oocyte to develop in a pseudopregnant female foster animal. The FDRG cDNA sequences of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632 can be introduced as a transgene into the genome of a non-human animal. Intronic sequences and polyadenylation signals can also be included in the transgene to increase the efficiency of expression of the transgene. A tissue-specific regulatory sequence(s) can be operably linked to the FDRG transgene to direct expression of FDRG protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the FDRG transgene in its genome and/or expression of FDRG mRNA in tissues or cells of the animals. A transgenic founder animal can then be used to breed additional animals carrying the transgene. Moreover, transgenic animals carrying a transgene encoding FDRG can further be bred to other transgenic animals carrying other transgenes.

To create a homologous recombinant animal, a vector is prepared which contains at least a portion of a FDRG gene into which a deletion, addition or substitution has been introduced to thereby alter, e.g., functionally disrupt, the FDRG gene. The FDRG gene can be a human gene (e.g., the cDNA of SEQ ID NO:1, SEQ ID NO:3 or SEQ ID NO:4), but more preferably, is a non-human homologue of a human FDRG gene. For example, a mouse FDRG gene (e.g., the cDNA of SEQ ID NO:11, SEQ ID NO:13 or SEQ ID NO:14) can be used to construct a homologous recombination vector suitable for altering an endogenous FDRG gene in the mouse genome. In a preferred embodiment, the vector is designed such that, upon homologous recombination, the endogenous FDRG gene is functionally disrupted (i.e., no longer encodes a functional protein; also referred to as a “knock out” vector). Alternatively, the vector can be designed such that, upon homologous recombination, the endogenous FDRG gene is mutated or otherwise altered but still encodes functional protein (e.g., the upstream regulatory region can be altered to thereby alter the expression of the endogenous FDRG protein). In the homologous recombination vector, the altered portion of the FDRG gene is flanked at its 5′ and 3′ ends by additional nucleic acid of the FDRG gene to allow for homologous recombination to occur between the exogenous FDRG gene carried by the vector and an endogenous FDRG gene in an embryonic stem cell. The additional flanking FDRG nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced FDRG gene has homologously recombined with the endogenous FDRG gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT Publication Numbers WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The FDRG nucleic acid molecules, FDRG proteins, and anti-FDRG antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a FDRG protein or anti-FDRG antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials); and d) methods of treatment (e.g., therapeutic and prophylactic methods as well as such methods in the context of pharmacogenetics). As described herein, a FDRG protein of the invention has one or more of the following activities: (i) interaction of a FDRG protein in the extracellular milieu with a non-FDRG protein molecule (e.g., a FDRG receptor) on the surface of the same cell which secreted the FDRG protein molecule; (ii) interaction of a FDRG protein in the extracellular milieu with a non-FDRG protein molecule (e.g., a FDRG receptor) on the surface of a different cell from that which secreted the FDRG protein molecule; (iii) complex formation between a FDRG protein and a FDRG receptor; (iv) complex formation between a FDRG protein and non-FDRG receptor; (v) modulation of signal transduction via a cell surface protein (e.g., a FDRG receptor); (vi) modulation of the expression of adipogenic genes; (vii) modulation of a PPARγ function or modulation by a PPARγ function; (viii) modulation of a cellular response to a PPARγ ligand (e.g., thiazolidinediones) and (ix) interaction of a FDRG protein with a second protein in the extracellular milieu (e.g., a FDRG binding protein).

Accordingly, a FDRG protein, FDRG modulator, or FDRG receptor modulator can be used in, for example, (1) activation of a FDRG-dependent signal transduction pathway; (2) modulation of angiogenesis; (3) modulation of a hematopoiesis; (4) modulation of the development or differentiation of a FDRG-expressing cell (e.g., mediation of growth and/or differentiation of adipocytes, for example, white adipocytes or brown adipocytes); (5) modulation of the proliferation, development or differentiation of a non-FDRG-expressing cell; (6) modulation of the homeostasis of a FDRG-expressing cell; (7) modulation of insulin sensitivity and/or insulin responsiveness; (8) modulation of insulin secretion (9) modulation of cell recruitment; (10) modulation of the homeostasis of a non-FDRG-expressing cell; (11) modulation of glucose metabolism; (12) maintenance of energy homeostasis (e.g., regulating the balance and/or imbalance between energy storage and energy expenditure, for example, increasing/decreasing energy expenditure); (13) regulation of adaptive thermogenesis (e.g., regulation of the biogenesis of mitochondria, regulation of the expression of mitocrondrial enzymes, regulation of expression of uncoupling proteins; (14) regulation of adiposity; (15) regulation of lipolysis and/or lipogenesis; (16) modulation of the efficiency of energy storage; (17) regulation of appetite; and (18) regulation of vasculogenesis (e.g., blood vessel formation or vascular remodeling); (19) regulation of tumor angiogenesis; (20) regulation of would healing; (expansion/reduction of tumor mass; and (21) expansion/reduction of fat mass.

The isolated nucleic acid molecules of the invention can be used, for example, to express FDRG protein (e.g., via a recombinant expression vector in a host cell in gene therapy applications), to detect FDRG mRNA (e.g., in a biological sample) or a genetic alteration in a FDRG gene, and to modulate FDRG activity, as described further below. In addition, the FDRG proteins can be used to screen drugs or compounds which modulate the FDRG activity as well as to treat disorders characterized by insufficient or excessive production of FDRG protein or production of FDRG protein forms which have decreased or aberrant activity compared to FDRG wild type protein (e.g., obesity and/or obesity-related disorders, diabetes and/or diabetes-related disorders, metabolic disorders, vascular disorders, differentiative or developmental disorders, proliferative disorders or cell recruitment disorders). Moreover, soluble forms of the FDRG protein can be used to bind membrane-bound FDRG receptors and influence bioavailability of such a receptors cognate ligand. In addition, the anti-FDRG antibodies of the invention can be used to detect and isolate FDRG proteins and modulate FDRG activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to FDRG proteins or have a stimulatory or inhibitory effect on, for example, FDRG expression or FDRG activity.

In one embodiment, the invention provides assays for screening candidate or test compounds which bind to or modulate the activity of a FDRG protein or polypeptide or biologically active portion thereof. In another embodiment, the invention provides assays for screening candidate or test compounds which bind to or modulate the activity of a FDRG receptor.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage ((Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In one embodiment, an assay is a cell-based assay in which a cell which expresses FDRG is contacted with a test compound and the ability of the test compound to modulate FDRG expression and/or activity determined. Determining the ability of the compound to modulate FDRG expression can be accomplished, for example, by detecting the presence or absence or amount of a FDRG transcript of protein (e.g., using a probe based on the nucleotide sequences of the present invention or an anti-FDRG antibody). Determining the ability of the compound to modulate FDRG activity can be accomplished, for example, by detecting FDRG activity in cellular supernatants (e.g., contacting a second cell with the supernatants).

In another embodiment, an assay is a cell-based assay in which a cell which expresses a FDRG receptor on the cell surface is contacted with a test compound and the ability of the test compound to bind to a FDRG receptor determined. The cell, for example, can be of mammalian origin or a yeast cell. Determining the ability of the test compound to bind to a FDRG receptor can be accomplished, for example, by coupling the test compound with a radioisotope or enzymatic label such that binding of the test compound to the FDRG receptor can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with a FDRG receptor without the labeling of any of the interactants. For example, a microphysiometer can be used to detect the interaction of a test compound with a FDRG receptor without the labeling of either the test compound or the receptor. McConnell, H. M. et al. (1992) Science 257:1906-1912. As used herein, a “microphysiometer” (e.g., Cytosensorm) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses a FDRG receptor on the cell surface with a FDRG protein or biologically-active portion thereof, to form an assay mixture, contacting the assay mixture with a test compound, and determining the ability of the test compound to interact with a FDRG receptor, wherein determining the ability of the test compound to interact with a FDRG receptor comprises determining the ability of the test compound to preferentially bind to the FDRG receptor as compared to the ability of FDRG, or a biologically active portion thereof, to bind to the receptor. Alternatively, the assay can comprise determining the ability of the test compound to modulate a cellular activity of FDRG and/or an FDRG receptor.

Determining the ability of the FDRG protein to bind to or interact with a FDRG target molecule, or to modulate a FDRG activity, can be accomplished by one of the methods described above for determining direct binding. In a preferred embodiment, determining the ability of the FDRG protein to bind to or interact with a FDRG target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a FDRG-responsive regulatory element operatively linked to a nucleic acid encoding a detectable marker, e.g., luciferase), or detecting a cellular response, for example, expression of a differentiated phenotype.

In yet another embodiment, an assay of the present invention is a cell-free assay in which a FDRG protein or biologically active portion thereof is contacted with a test compound and the ability of the test compound to bind to the FDRG protein or biologically active portion thereof is determined. Binding of the test compound to the FDRG protein can be determined either directly or indirectly as described above. In a preferred embodiment, the assay includes contacting the FDRG protein or biologically active portion thereof with a known compound which binds FDRG to form an assay mixture, contacting the assay mixture with a test compound, and determining the ability of the test compound to interact with a FDRG protein, wherein determining the ability of the test compound to interact with a FDRG protein comprises determining the ability of the test compound to preferentially bind to FDRG or biologically active portion thereof as compared to the known compound.

In another embodiment, the assay is a cell-free assay in which FDRG protein or biologically active portion thereof is contacted with a test compound and the ability of the test compound to modulate (e.g., stimulate or inhibit) the activity of the FDRG protein or biologically active portion thereof is determined. Determining the ability of the test compound to modulate the activity of FDRG can be accomplished, for example, by determining the ability of the FDRG protein to bind to a FDRG target molecule (e.g., a FDRG binding protein) by one of the methods described above for determining direct binding. Determining the ability of the FDRG protein to bind to a FDRG target molecule can also be accomplished using a technology such as real-time Biomolocular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of FDRG can be accomplished by determining the ability of the FDRG protein to further modulate a FDRG target molecule. For example, the activity of the target molecule on a receptor can be determined as previously described.

In yet another embodiment, the cell-free assay involves contacting the FDRG protein or biologically active portion thereof with a known compound which binds FDRG to form an assay mixture, contacting the assay mixture with a test compound, and determining the ability of the test compound to interact with a FDRG protein, wherein determining the ability of the test compound to interact with a FDRG protein comprises determining the ability of the FDRG protein to preferentially bind to or modulate the activity of a FDRG target molecule.

The cell-free assays of the present invention are amenable to use of both soluble and/or membrane-bound forms of isolated proteins (e.g. FDRG proteins or biologically active portions thereof or FDRG target molecules). In the case of cell-free assays in which a membrane-bound form an isolated protein is used (e.g., a FDRG target molecule or receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either FDRG or its target molecule to facilitate separation of complexed from uncomplexed forms of one or both of the proteins, as well as to accommodate automation of the assay. Binding of a test compound to FDRG, or interaction of FDRG with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/FDRG fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or FDRG protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of FDRG binding or activity determined using standard techniques.

Other techniques for immobilizing proteins on matrices can also be used in the screening assays of the invention. For example, either FDRG or its target molecule can be immobilized utilizing conjugation of biotin and streptavidin. Biotinylated FDRG or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with FDRG or target molecules but which do not interfere with binding of the FDRG protein to its target molecule can be derivatized to the wells of the plate, and unbound target or FDRG trapped in the wells by antibody conjugation. Methods for detecting such complexes, in addition to those described above for the GST-immobilized complexes, include immunodetection of complexes using antibodies reactive with the FDRG or target molecule, as well as enzyme-linked assays which rely on detecting an enzymatic activity associated with the FDRG or target molecule.

In another embodiment, modulators of FDRG expression are identified in a method wherein a cell is contacted with a candidate compound and the expression of FDRG mRNA or protein in the cell is determined. The level of expression of FDRG mRNA or protein in the presence of the candidate compound is compared to the level of expression of FDRG mRNA or protein in the absence of the candidate compound. The candidate compound can then be identified as a modulator of FDRG expression based on this comparison. For example, when expression of FDRG mRNA or protein is greater (statistically significantly greater) in the presence of the candidate compound than in its absence, the candidate compound is identified as a stimulator of FDRG mRNA or protein expression. Alternatively, when expression of FDRG mRNA or protein is less (statistically significantly less) in the presence of the candidate compound than in its absence, the candidate compound is identified as an inhibitor of FDRG mRNA or protein expression. The level of FDRG mRNA or protein expression in the cells can be determined by methods described herein for detecting FDRG mRNA or protein.

In yet another aspect of the invention, the FDRG proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with FDRG (“FDRG-binding proteins” or “FDRG-bp”) and modulate FDRG activity. Such FDRG-binding proteins are also likely to be involved in the propagation of signals by the FDRG proteins as, for example, downstream elements of a FDRG-mediated signaling pathway. Alternatively, such FDRG-binding proteins are likely to be cell-surface molecules associated with non-FDRG expressing cells, wherein such FDRG-binding proteins are involved in secondary cytokine production.

The two-hybrid system is based on the modular nature of most transcription factors, which consist of separable DNA-binding and activation domains. Briefly, the assay utilizes two different DNA constructs. In one construct, the gene that codes for FDRG is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a FDRG-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with FDRG.

This invention further pertains to novel agents identified by the above-described screening assays. Accordingly, it is within the scope of this invention to further use an agent identified as described herein in an appropriate animal model. For example, an agent identified as described herein (e.g., a FDRG modulating agent, an antisense FDRG nucleic acid molecule, a FDRG-specific antibody, or a FDRG binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the FDRG, sequences, described herein, can be used to map the location of the FDRG genes, respectively, on a chromosome. The mapping of the FDRG sequences to chromosomes is an important first step in correlating these sequences with genes associated with disease.

Briefly, FDRG genes can be mapped to chromosomes by preparing PCR primers (preferably 15-25 bp in length) from the FDRG sequences. Computer analysis of the FDRG sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the FDRG sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the FDRG sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes (e.g., non coding sequences of the genomic DNA described in Example 7 herein). Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the FDRG gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The FDRG sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the FDRG sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The FDRG sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from FDRG sequences described herein is used to generate a unique identification database for an individual, those same reagents can later be used to identify tissue from that individual. Using the unique identification database, positive identification of the individual, living or dead, can be made from extremely small tissue samples.

3. Use of Partial FDRG Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the FDRG sequences or portions thereof, e.g., fragments derived from the noncoding regions of SEQ ID NO:1 having a length of at least 20 bases, preferably at least 30 bases.

The FDRG sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such FDRG probes can be used to identify tissue by species and/or by organ type.

In a similar fashion, these reagents, e.g., FDRG primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining FDRG protein and/or nucleic acid expression as well as FDRG activity, in the context of a biological sample (e.g., blood, serum, cells, tissue) to thereby determine whether an individual is afflicted with a disease or disorder, or is at risk of developing a disorder, associated with aberrant FDRG expression or activity. The invention also provides for prognostic (or predictive) assays for determining whether an individual is at risk of developing a disorder associated with FDRG protein, nucleic acid expression or activity. For example, mutations in a FDRG gene can be assayed in a biological sample. Such assays can be used for prognostic or predictive purpose to thereby phophylactically treat an individual prior to the onset of a disorder characterized by or associated with FDRG protein, nucleic acid expression or activity.

Another aspect of the invention pertains to monitoring the influence of agents (e.g., drugs, compounds) on the expression or activity of FDRG in clinical trials.

These and other agents are described in further detail in the following sections.

1. Diagnostic Assays

An exemplary method for detecting the presence or absence of FDRG in a biological sample involves obtaining a biological sample from a test subject and contacting the biological sample with a compound or an agent capable of detecting FDRG protein or nucleic acid (e.g., mRNA, genomic DNA) that encodes FDRG protein such that the presence of FDRG is detected in the biological sample. A preferred agent for detecting FDRG mRNA or genomic DNA is a labeled nucleic acid probe capable of hybridizing to FDRG mRNA or genomic DNA. The nucleic acid probe can be, for example, a full-length FDRG nucleic acid, such as the nucleic acid of SEQ ID NO:1, SEQ ID NO:3, SEQ ID NO:4, SEQ ID NO:11, SEQ ID NO:13, SEQ ID NO:14, the DNA insert of the plasmid deposited with ATCC as Accession Number 98632, or a portion thereof, such as an oligonucleotide of at least 15, 30, 50, 100, 250 or 500 nucleotides in length and sufficient to specifically hybridize under stringent conditions to FDRG mRNA or genomic DNA. Other suitable probes for use in the diagnostic assays of the invention are described herein.

A preferred agent for detecting FDRG protein is an antibody capable of binding to FDRG protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect FDRG mRNA, protein, or genomic DNA in a biological sample in vitro as well as in vivo. For example, in vitro techniques for detection of FDRG mRNA include Northern hybridizations and in situ hybridizations. In vitro techniques for detection of FDRG protein include enzyme linked immunosorbent assays (ELISAs), Western blots, immunoprecipitations and immunofluorescence. In vitro techniques for detection of FDRG genomic DNA include Southern hybridizations. Furthermore, in vivo techniques for detection of FDRG protein include introducing into a subject a labeled anti-FDRG antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting FDRG protein, mRNA, or genomic DNA, such that the presence of FDRG protein, mRNA or genomic DNA is detected in the biological sample, and comparing the presence of FDRG protein, mRNA or genomic DNA in the control sample with the presence of FDRG protein, mRNA or genomic DNA in the test sample.

The invention also encompasses kits for detecting the presence of FDRG in a biological sample. For example, the kit can comprise a labeled compound or agent capable of detecting FDRG protein or mRNA in a biological sample; means for determining the amount of FDRG in the sample; and means for comparing the amount of FDRG in the sample with a standard. The compound or agent can be packaged in a suitable container. The kit can further comprise instructions for using the kit to detect FDRG protein or nucleic acid.

2. Prognostic Assays

The diagnostic methods described herein can furthermore be utilized to identify subjects having or at risk of developing a disease or disorder associated with aberrant FDRG expression or activity. For example, the assays described herein, such as the preceding diagnostic assays or the following assays, can be utilized to identify a subject having or at risk of developing a disorder associated with FDRG protein, nucleic acid expression or activity such as a proliferative disorder, a differentiative or developmental disorder, a cell migration disorder or a hematopoietic disorder. Thus, the present invention provides a method for identifying a disease or disorder associated with aberrant FDRG expression or activity in which a test sample is obtained from a subject and FDRG protein or nucleic acid (e.g., mRNA, genomic DNA) is detected, wherein the presence of FDRG protein or nucleic acid is diagnostic for a subject having or at risk of developing a disease or disorder associated with aberrant FDRG expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant FDRG expression or activity. For example, such methods can be used to determine whether a subject can be effectively treated with an agent for a disorder, such as a proliferative disorder, a differentiative or developmental disorder, a cell migration or recruitment disorder, or a hematopoietic disorder. Thus, the present invention provides methods for determining whether a subject can be effectively treated with an agent for a disorder associated with aberrant FDRG expression or activity in which a test sample is obtained and FDRG protein or nucleic acid is detected (e.g., wherein the presence of FDRG protein or nucleic acid is diagnostic for a subject that can be administered the agent to treat a disorder associated with aberrant FDRG expression or activity.)

The methods of the invention can also be used to detect genetic alterations in a FDRG gene, thereby determining if a subject with the altered gene is at risk for a disorder characterized by aberrant differentiation, aberrant angiogenesis, an aberrant proliferative response or an aberrant hematopoietic response. In preferred embodiments, the methods include detecting, in a sample of cells from the subject, the presence or absence of a genetic alteration characterized by at least one of an alteration affecting the integrity of a gene encoding a FDRG protein, or the mis-expression of the FDRG gene. For example, such genetic alterations can be detected by ascertaining the existence of at least one of 1) a deletion of one or more nucleotides from a FDRG gene; 2) an addition of one or more nucleotides to a FDRG gene; 3) a substitution of one or more nucleotides of a FDRG gene, 4) a chromosomal rearrangement of a FDRG gene; 5) an alteration in the level of a messenger RNA transcript of a FDRG gene, 6) aberrant modification of a FDRG gene, such as of the methylation pattern of the genomic DNA, 7) the presence of a non-wild type splicing pattern of a messenger RNA transcript of a FDRG gene, 8) a non-wild type level of a FDRG protein, 9) allelic loss of a FDRG gene, and 10) inappropriate post-translational modification of a FDRG protein. As described herein, there are a large number of assay techniques known in the art which can be used for detecting alterations in a FDRG gene. A preferred biological sample is serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the FDRG gene (see Abravaya et al. (1995) Nucleic Acids Res 0.23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a FDRG gene under conditions such that hybridization and amplification of the FDRG gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a FDRG gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in FDRG can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in FDRG can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential ovelapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the FDRG gene and detect mutations by comparing the sequence of the sample FDRG with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays (Naeve et al.(1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the FDRG gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type FDRG sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in FDRG cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a FDRG sequence, e.g., a wild-type FDRG sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in FDRG genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control FDRG nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

As described in Example 8 herein, the human FDRG gene was localized to chromosome 19p13.3, 7.8 cR distal of marker AFMA135XB9 and 2.4 cR proximal of marker RP_S28_{—}1. This region is close to ATHS (atherosclerosis susceptibility) and ML4 (sialolipidosis). Accordingly, in another embodiment, FDRG nucleic acid molecules of the present invention can be used in diagnostic assays (e.g., genetic linkage analysis) of patients at risk or suspected to be at risk for ATHS and/or ML4.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a FDRG gene.

Furthermore, any cell type or tissue in which FDRG is expressed may be utilized in the prognostic assays described herein.

3. Monitoring of Effects During Clinical Trials

Monitoring the influence of agents (e.g., drugs, compounds) on the expression or activity of FDRG (e.g., activation of a FDRG-dependent signal transduction pathway; modulation of angiogenesis or cellular proliferation; modulation of cell migration or recruitment; or modulation of a hematopoietic response) can be applied not only in basic drug screening, but also in clinical trials. For example, the effectiveness of an agent determined by a screening assay as described herein to increase FDRG gene expression, protein levels, or upregulate FDRG activity, can be monitored in clinical trials of subjects exhibiting decreased FDRG gene expression, protein levels, or downregulated FDRG activity. Alternatively, the effectiveness of an agent determined by a screening assay to decrease FDRG gene expression, protein levels, or downregulate FDRG activity, can be monitored in clinical trials of subjects exhibiting increased FDRG gene expression, protein levels, or upregulated FDRG activity. In such clinical trials, the expression or activity of FDRG and, preferably, other genes that have been implicated in, for example, a proliferative disorder can be used as a “read out” or markers of the proliferation of a particular cell.

For example, and not by way of limitation, genes, including FDRG, that are modulated in cells by treatment with an agent (e.g., compound, drug or small molecule) which modulates FDRG activity (e.g., identified in a screening assay as described herein) can be identified. Thus, to study the effect of agents on proliferative disorders, developmental disorder, or hematopoietic disorder, for example, in a clinical trial, cells can be isolated and RNA prepared and analyzed for the levels of expression of FDRG and other genes implicated in the proliferative disorder, developmental disorder, or hematopoietic disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of FDRG or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a FDRG protein, mRNA, or genomic DNA in the preadministration sample; (iii) obtaining one or more post-administration samples from the subject; (iv) detecting the level of expression or activity of the FDRG protein, mRNA, or genomic DNA in the post-administration samples; (v) comparing the level of expression or activity of the FDRG protein, mRNA, or genomic DNA in the pre-administration sample with the FDRG protein, mRNA, or genomic DNA in the post administration sample or samples; and (vi) altering the administration of the agent to the subject accordingly. For example, increased administration of the agent may be desirable to increase the expression or activity of FDRG to higher levels than detected, i.e., to increase the effectiveness of the agent. Alternatively, decreased administration of the agent may be desirable to decrease expression or activity of FDRG to lower levels than detected, i.e. to decrease the effectiveness of the agent. According to such an embodiment, FDRG expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant FDRG expression or activity. With regards to both prophylactic and therapeutic methods of treatment, such treatments may be specifically tailored or modified, based on knowledge obtained from the field of pharmacogenomics.

“Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the FDRG molecules of the present invention or FDRG modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant FDRG expression or activity, by administering to the subject an agent which modulates FDRG expression or at least one FDRG activity. Subjects at risk for a disease which is caused or contributed to by aberrant FDRG expression or activity can be identified by, for example, any or a combination of diagnostic or prognostic assays as described herein. Administration of a prophylactic agent can occur prior to the manifestation of symptoms characteristic of the FDRG aberrancy, such that a disease or disorder is prevented or, alternatively, delayed in its progression. Depending on the type of FDRG aberrancy, for example, a FDRG agonist or FDRG antagonist agent can be used for treating the subject. The appropriate agent can be determined based on screening assays described herein.

2. Therapeutic Methods

Another aspect of the invention pertains to methods of modulating FDRG expression or activity for therapeutic purposes. The modulatory method of the invention involves contacting a cell with an agent that modulates one or more of the activities of FDRG protein activity associated with the cell. An agent that modulates FDRG protein activity can be an agent as described herein, such as a nucleic acid or a protein, a naturally-occurring target molecule of a FDRG protein, a peptide, a FDRG peptidomimetic, or other small molecule. In one embodiment, the agent stimulates one or more FDRG protein activity. Examples of such stimulatory agents include active FDRG protein and a nucleic acid molecule encoding FDRG that has been introduced into the cell. In another embodiment, the agent inhibits one or more FDRG protein activity. Examples of such inhibitory agents include antisense FDRG nucleic acid molecules and anti-FDRG antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a FDRG protein or nucleic acid molecule. In one embodiment, the method involves administering an agent (e.g., an agent identified by a screening assay described herein), or combination of agents that modulates (e.g., upregulates or downregulates) FDRG expression or activity. In another embodiment, the method involves administering a FDRG protein or nucleic acid molecule as therapy to compensate for reduced or aberrant FDRG expression or activity.

A preferred embodiment of the present invention involves a method for treatment of a disease or disorder associated with a FDRG protein which includes the step of administering a therapeutically effective amount of an antibody to a FDRG protein to a subject. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

Stimulation of FDRG activity is desirable in situations in which FDRG is abnormally downregulated and/or in which increased FDRG activity is likely to have a beneficial effect. One example of such a situation is where a subject has a disorder characterized by aberrant proliferative disorder (e.g., cancer). Yet another example of such a situation is where a subject has a disorder characterized by an aberrant hematopoietic response. Yet another example of such a situation is where a subject has a disorder characterized by aberrant differentiation or development or aberrant cell migration.

3. Pharmacogenomics

The FDRG molecules of the present invention, as well as agents, or modulators which have a stimulatory or inhibitory effect on FDRG activity (e.g., FDRG gene expression) as identified by a screening assay described herein can be administered to individuals to treat (prophylactically or therapeutically) disorders (e.g, proliferative disorders or developmental disorders) associated with aberrant FDRG activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a FDRG molecule or FDRG modulator as well as tailoring the dosage and/or therapeutic regimen of treatment with a FDRG molecule or FDRG modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a FDRG protein or FDRG receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a FDRG molecule or FDRG modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a FDRG molecule or FDRG modulator, such as a modulator identified by one of the exemplary screening assays described herein.

This invention is further illustrated by the following examples which should not be construed as limiting. The contents of all references, patents and published patent applications cited throughout this application are hereby incorporated by reference.

### EXAMPLES

### Example 1

### Isolation and Characterization of Human FDRG cDNAs

In this example, the isolation and characterization of the gene encoding human FDRG (also referred to as “TANGO115” or “T115”) is described.

Isolation of Human FDRG cDNA

A human FDRG cDNA was isolated from a human aortic endothelial cDNA library. To construct the library, three micrograms of poly A⁺ RNA were isolated from human aortic endothelial cells, reverse transcribed, and used to synthesize a cDNA library using the Superscript cDNA Synthesis kit™ (Gibco BRL; Gaithersburg, Md.). Complementary DNA was directionally cloned into the expression plasmid pMET7 using the SalI and NotI sites in the polylinker to construct a plasmid library. Transformants were picked and amplified for single-pass sequencing. Additionally, human aortic endothelial cDNA was ligated into the Sall/NotI sites of the ZIPLOX™ vector (Gibco BRL) for construction of a lambda phage cDNA library.

A partial cDNA clone encoding FDRG was identified from the above-described cDNA library using the following method. First, each sequence was checked to determine if it was a bacterial, ribosomal, or mitochondrial contaminant. Such sequences were excluded from the subsequent analysis. Second, sequence artifacts, such as vector and repetitive elements, were masked and/or removed from each sequence. Third, the remaining sequences were searched against a copy of the GenBank nucleotide database using the BLASTN™ program (BLASTN 1.3 MP: Altschul et al., J. Mol. Bio. 215:403, 1990). Fourth, the sequences were analyzed against a non-redundant protein database with the BLASTX™ program, which translates a nucleic acid sequence in all six frames and compares it against available protein databases (BLASTX 1.3 MP:Altschul et al, supra). This protein database is a combination of the Swiss-Prot, PIR, and NCBI GenPept protein databases.

The original first pass sequence of the partial cDNA clone showed homology to angiopoietin 2 using the BLASTX™ program. Additional screening of the phage library led to the isolation of a full length clone for human FDRG. The nucleotide sequence and predicted amino acid sequence are shown in FIG. 1 (corresponding to SEQ ID NO:1 and SEQ ID NO:2, respectively.) The FDRG protein (corresponding to amino acids 1-406 of the predicted amino acid sequence, SEQ ID NO:2) shows 31.0% identity to the human ficolin and 29.0% identity to human angiopoietin 2 (see FIG. 3). Percent identity was calculated using the Gap program from GCG (Wisconsin Genetics Computer Group) which incorporates the Needleman-Wunsch alignment algorithm, Needleman and Wunsch (1970) J. Mol. Biol. 48:444-453, using a gap weight of 12 and a length weight of 4.

This human FDRG protein contains a C-terminal fibrinogen-like domain (corresponding to amino acids 186-399 of the predicted amino acid sequence, SEQ ID NO:2 and amino acids 161-376 of SEQ ID NO:5) and a signal sequence (corresponding to amino acids 1-25 of the predicted amino acid sequence, SEQ ID NO:2) which is cleaved to form a mature FDRG protein (corresponding to amino acids 1-381 of SEQ ID NO:5).

### Example 2

### Distribution of FDRG mRNA in Human Tissues

Northern Blot Analysis

The expression of FDRG was analyzed using Northern blot hybridization. For analysis of human FDRG, the entire FDRG insert was isolated from the plasmid following SalI/NotI digestion and used as a probe. The probe DNA was radioactively labeled with ³²P-dCTP using the Prime-It kit™ (Stratagene, La Jolla, Calif.) according to the instructions of the supplier. Filters containing human mRNA (MTNI and MTNII from Clontech, Palo Alto, Calif.) were probed in ExpressHyb™ hybridization solution (Clontech) and washed at high stringency according to manufacturer's recommendations.

An approximately 2.0 kb FDRG transcript was observed at variable levels in all tissues examined (spleen, thymus, prostate, testes, ovary, small intestine, colon, heart, brain, placenta, lung, liver, skeletal muscle, kidney and pancreas) with the sole exception of peripheral blood leukocytes. The highest levels of FDRG expression were observed in the placenta.

Northern analysis was also performed on 10 μg of total RNA prepared from the following surgical specimens: placenta, heart, kidney, liver and fat. Again, a single 2.0 kb transcript was observed that was highly enriched in white fat and placenta.

### Example 3

### Screening of Mouse Tissues for FDRG Binding Sites

This Example describes the use of cell supernatant containing AP-hFDRG to screen tissue sections of mouse embryos and of whole mice for FDRG binding sites.

Screening of tissue section with supernatant containing alkaline phosphatase (AP) fusion protein was done as described by Cheng and Flanagan (1994) Cell 79:157-168. Briefly, fresh frozen sections (8 μm) of mouse embryos (day 14.5 of prenatal development, E 14.5) and of whole mice (postnatal day 1.5, P 1.5) were prepared and rinsed in HBHA (Hank's balanced salt solution supplemented with 20 mM Hepes, pH 7, 0.05% BSA and 0.1% sodium azide). Subsequently tissue sections were incubated for 1 h at room temperature with supernatant containing AP-hFDRG (alkaline phosphatase fused to the N-terminus of human FDRG, respectively) or AP at a concentration of 5 nM. After the incubation, tissue sections were washed six times in HBHA, fixed in a solution containing 60% acetone, 3% formaldehyde and 20 mM Hepes, pH 7.5, washed three times in HBS (20 mM Hepes, pH 7.5, 150 mM NaCl) and heated for 30 min at 65° C. to inactivate endogenous alkaline phosphatase activity. Bound AP fusion protein was detected by developing sections in BCIP/NBT substrate solution (100 mM Tris-HCl, pH 9.5, 100 mM NaCl, 5 mM MgCl, 0.17 mg/ml BCIP and 0.33 mg/ml NBT).

AP-hFDRG but not AP protein bound to the epidermis in both E 14.5 and P 1.5 sections and to adipose tissue in P1.5 sections. Binding of APhFDRG but not AP was also observed to epidermis and adipose tissue of adult mice. These results suggest that cells in epidermis and adipose tissue harbour binding sites for FDRG.

### Example 4

### Isolation and Characterization of Murine FDRG cDNAs

A murine FDRG cDNA (also referred to herein as “BK89” or “PPAR Gamma Angiopoietin-Related” or “PGAR”) was cloned by a PCR-based subractive hybridization method. The subtractive cloning strategy was designed to take advantage of the availability of cellular model systems and the specificity provided by the synthetic PPARγ ligands. Because fibroblastic cells that ectopically express PPARγ display a basal level of spontaneous adipose conversion, perhaps reflecting a low but significant level of ligand(s) produced endogenously or present in the media, two NIH3T3 cell lines were compared, one infected with a retrovirus containing an empty vector (e.g., an empty pBABE vector) and the other carrying pBABE-PPARγ (e.g., expressing PPARγ).

Briefly, BOSC23 cells were transiently transfected with pBabe-derived expression vectors and the resulting supernatants containing recombinant virus were transferred to the NIH3T3 fibroblasts, which were subsequently replated and selected in puromycin. The two cell lines were treated with 10 μg/ml pioglitazone (Upjohn), a PPARγligand, for a period of three hours, and PolyA+ RNA was purified from the cells. The double stranded cDNA resulting from reverse transcription was then digested and used for multiple rounds of subtractive hybridization and PCR amplification according to a PCR-Select™ DNA subtraction protocol (Clonetech).

The subtracted library was then screened by slot blot analysis for differentially regulated genes, and candidates were confirmed by northern blotting. The enriched cDNAs were cloned into pCR2.1 vector (Invitrogen) and screened by slot blot analysis for differentially regulated genes. Plasmid DNA from three hundred random clones were spotted onto nylon membranes using a vacuum manifold apparatus and hybridized with [α-³²P] labeled cDNA probes prepared by reverse transcription from the two PolyA+ RNA samples used above. Northern blots were then performed on those clones judged to be differentially regulated on the slot blots. Twenty μg of total RNA prepared for the subtraction experiment was fractionated in a 1% agarose-formaldehyde gel, transferred onto nylon, and hybridized with the EcoRI-excised inserts labeled by random-primed labeling with [α-³²P] dCTP as described (Tontonoz et al. (1994) Cell 79:1147-1156). FDRG was one of the first ten or so clones to be isolated from this screen.

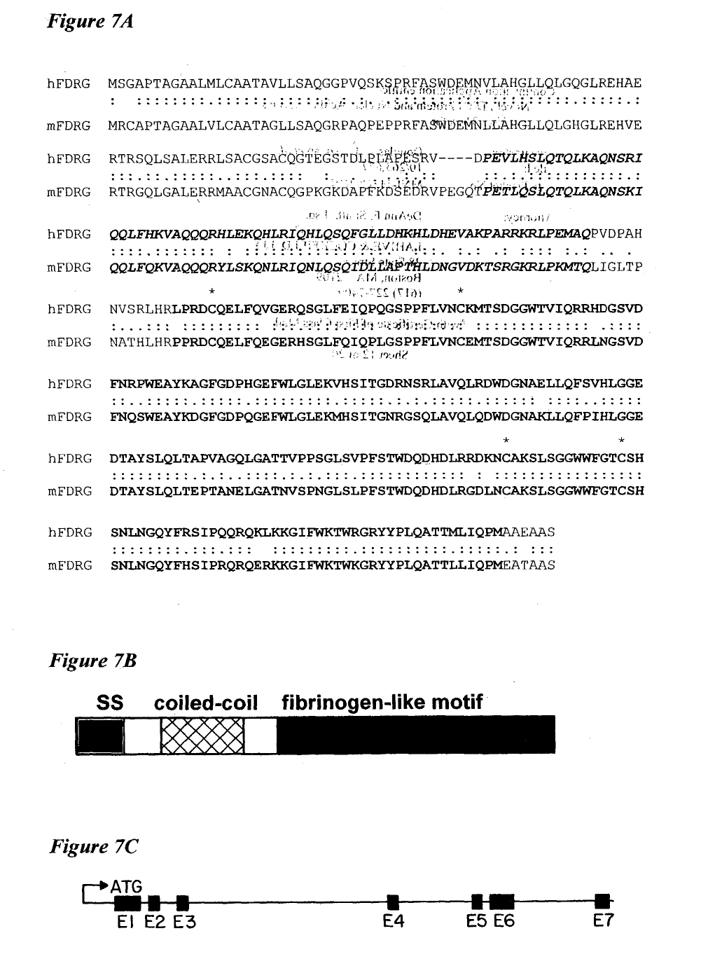

The full-length clone of murine FDRG was obtained from a 3T3-F442A adipocyte λZAPII cDNA library. The library screening, subcloning, and sequence analysis were done as described (Ausubel et al. (1991) Current Protocols in Molecular Biology, New York, John Wiley & Sons, Inc.). An open reading frame encoding 410 amino acid residues was identified predicting a 45 kDa protein. Hydrophobic sequences are present in the N-terminal regions of murine FDRG, consistent with secretory signal peptides. Sequence homology searches revealed that the C-terminal half of murine FDRG displays strong similarity to a family of proteins sharing the so-called fibrinogen-like motif, such as angiopoietins-1,2, fibrinogen α, β, and γ chains, tenascin, ficolin, and pT49. The N-terminal half, on the other hand, displays little homology to known proteins. The residues 50 to 150 are likely to form a coiled-coil quaternary structure, in common with most other members of the fibrinogen-like protein family, according to the results of a computer algorithm-based analysis of the primary structure. The presence of this motif suggests that FDRG may form multimeric structures or other higher order structures (Procopio et al. (1999) J. Biol. Chem. 274:30196-30201). The murine FDRG protein also contains three potential N-glycosylation sites, and four cysteines that may be available for intramolecular disulfide bonding. These cysteine residues are conserved not only between human and mouse FDRG, but are also conserved in angiopoietin-2 and ficolin, as shown in FIG. 3A-B. Human and mouse FDRG are ˜75% identical at the amino acid level (FIG. 7).

### Example 5

### Secretion of Murine FDRG From COS7 Cells

Vectors encoding untagged and tagged murine FDRG were constructed as follows. Briefly, forward and reverse primers were synthesized and used to amplify the full-length open reading frame of FDRG from pBluescript SK(+/−) by PCR. 5′ primers incorporate the native 5′ untranslated region upstream of the initiator methionine and 3′ primers include the nucleotide sequence encoding the YPYDVPDYA (hemagglutinin, HA) epitope (SEQ ID NO:24) followed by a termination codon.

For untagged FDRG, the following primers were used:

5′ primer: 5′ AATTAACCCTCACTAAAGGG 3′ (SEQ ID NO:18)

3′ primer: ACGCGTCGACTAATACGACTCACTATAGGGCG (SEQ ID NO:19)

For tagged FDRG, the following primers were used:

5′ primer: 5′ AATTAACCCTCACTAAAGGG 3′ (SEQ ID NO:18)

3′ primer: 5′ TACGCGTCGACCTAAGCGTAGTCTGGGACGTC GTATGGGTAAGAGGCTGCTGTAGCCTCCAT 3′ (SEQ ID NO:20)

Following amplification, The PCR products were cut with EcoRI/Sal I and were cloned into EcoRI/Xhol cut pcDNA 3.

HA-tagged murine FDRG (in pcDNA3, Invitrogen) was expressed in COS7 cells using transient transfection and detected the protein in the conditioned media by immunoblotting. COS7 cells were grown to 80% confluence in 10% Cosmic calf serum/DMEM and were transiently transfected with pcDNA or pcDNA-PGAR-HA using Superfect™ (Qiagen). After 24 hr, cells were washed with DMEM, and fresh serum-free medium was added. The conditioned medium was collected 48 hr post transfection and 0.22μ filtered To enhance the visibility of the signal, the conditioned media was concentrated ten-fold before loading by centrifugation in a 10 kDa MW cutoff spin colunn.

Cell extracts were prepared with RIPA lysis buffer as described (Ausubel et al., supra). Proteins were separated in a SDS-polyacrylamide gel, transferred to PVDF membranes, and incubated with a monoclonal antibody against the HA antigen (1:1000), washed, and then incubated with anti-mouse IgG conjugated with horseradish peroxidase (1:3000), followed by detection with ECLTM reagents). The epitope-tagged protein produced by the cells was observed to have a slightly higher molecular weight than the in vitro translate, consistent with posttranslational modifications such as glycosylation.

### Example 6

### Generation and Characterization of Epitope-Tagged FDRG Proteins Expression of Epitope-Tagged Human FDRG

A human FDRG flag epitope-tagged protein (huFDRG:flag) vector was constructed by PCR followed by ligation into an expression vector, pMET stop. The full-length open-reading frame was PCR amplified using a 5′ primer incorporating a Kozak sequence upstream of and including the initiator methionine and a 3′ primer including the nucleotide sequence encoding the DYKDDDDK flag epitope (SEQ ID NO:21) followed by a termination codon. The primer sequences are shown below:

5′ primer: 5′ TTTTTCAATTGACCGCCATGAGCGGTGCTCCGACG3′ (SEQID NO:22)

3′ primer: 5′ TTTTTGTCGACTTATCACTTGTCGTCGTCGTCCTTGTA GTCGGAGGCTGCCTCT-GCTGC 3′ (SEQ ID NO:23)

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, Gibco/BRL) was harvested and spun.

Conditioned medium was electroblotted onto a PVDF membrane (Novex) after separation by SDS-PAGE on 4-20% gradient gels and probed with an M2 anti-flag polyclonal antibody (1:500, Sigma) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Flag immunoreactivity appeared as a major band that migrated at an apparent molecular weight of 42 kDa as determined by MultiMark molecular weight markers (Novex) by SDS-PAGE.

Expression of Epitope-Tagged Murine FDRG

A murine HA-tagged FDRG expression construct was made as described in Example 5. The DNA construct was sequenced and then transiently transfected into HEK 293T cells in 150 mm plates using Lipofectamine (GIBCO/BRL) according to the manufacturer's protocol. Seventy-two hours post-transfection, the serum-free conditioned medium (OptiMEM, Gibco/BRL) was harvested and spun. Conditioned medium was electroblotted onto PVDF (Novex) after separation by SDS-PAGE on 4-20% gradient gels and probed with an anti-HA monoclonal antibody, HA.11 (1:500, Babco) followed by HRP conjugated sheep anti-mouse antibody (1:5000, Amersham), developed using chemiluminescent reagents (Renaissance, Dupont), and exposed to autoradiography film (Biomax MR2 film, Kodak). HA immunoreactivity appeared as a major band that migrated at 60 kDa as determined by MultiMark markers (Novex) by SDS-PAGE. A second fainter band was seen at 42 kDa.

Mock Transfected Supernatant

A mock supernatant was prepared by transfecting 293T cells with a pMET stop vector containing no insert. Seventy-two hours post-transfection, the serum-free conditioned medium (OptiMEM, Gibco/BRL) was harvested and spun. Conditioned media was electroblotted, developed, and exposed as described above. No specific bands were detected on either anti-flag or anti-HA blots.

### Example 7

### Isolation of FDRG Genomic DNA

Mouse genomic clones were isolated by screening a mouse 129SV BAC library (Research Genetics) by PCR. The primers used for screening were as follows: forward primer 5′-CTCTTAGCCTCCTCACTGGAG-3′ (SEQ ID NO:30), reverse primer 5′-CACAGTTAGCACCTGTGCATC-3′ (SEQ ID NO:31). Amplification conditions were initial denaturation at 95° C. for 2 min, 35 cycles of 95° C. for 30 s, 57° C. for 30 s, and 72° C. for 30 s, followed by a final extension at 72° C. for 10 min. The positive clone address was 313H16. The PGAR BAC clone was purified by DNA Megaprep Kit (Qiagen) and sequenced with PGAR-specific primers.

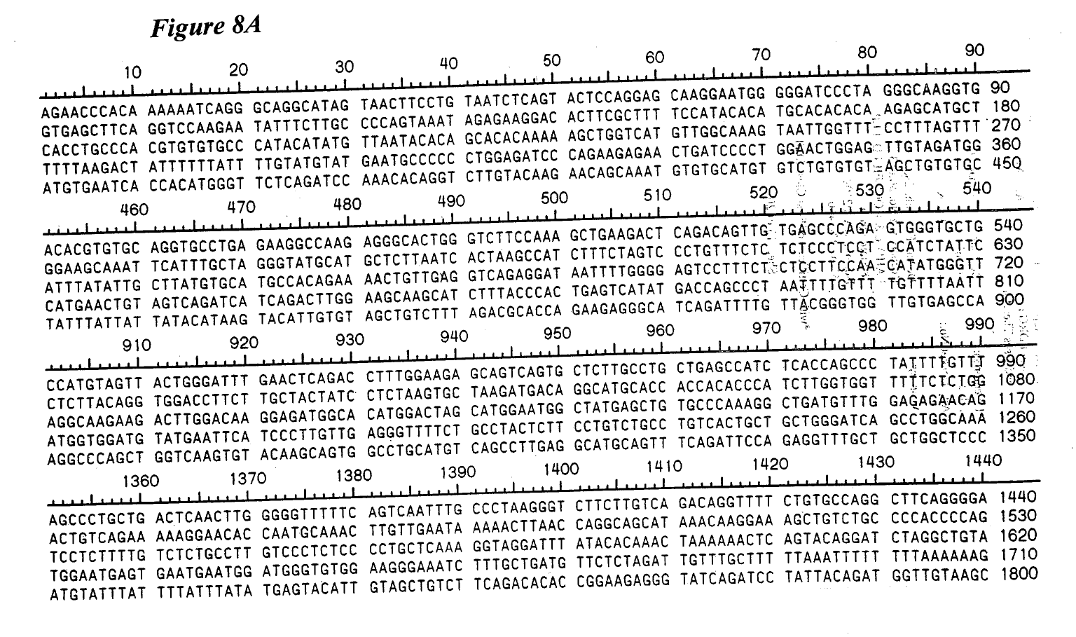

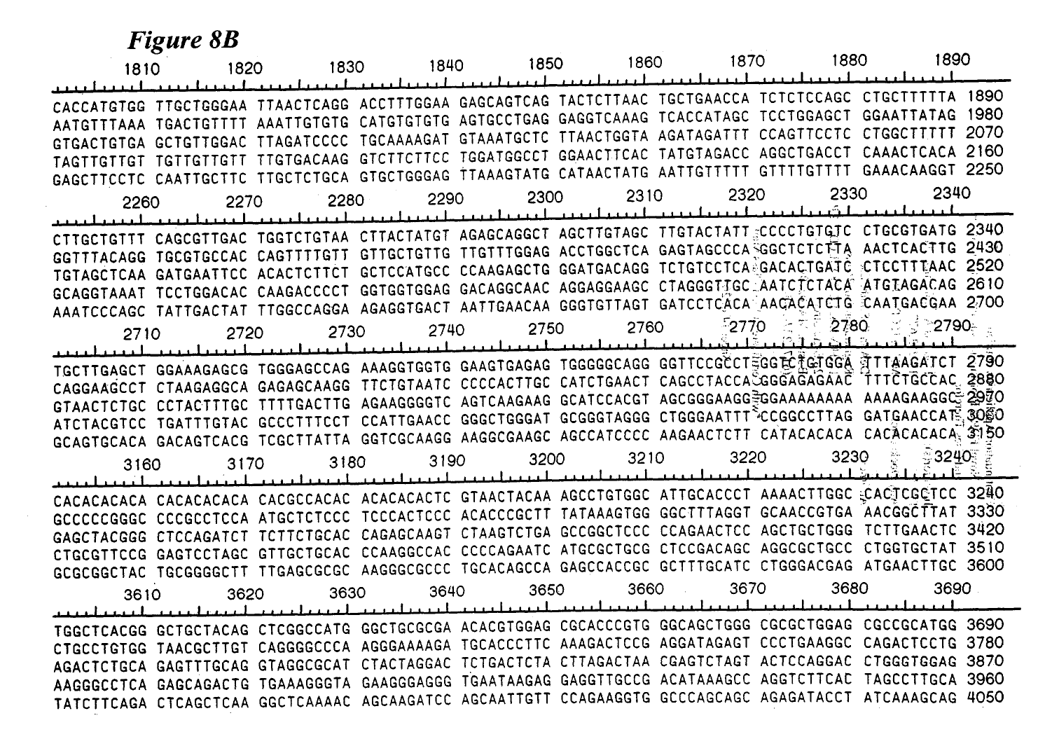

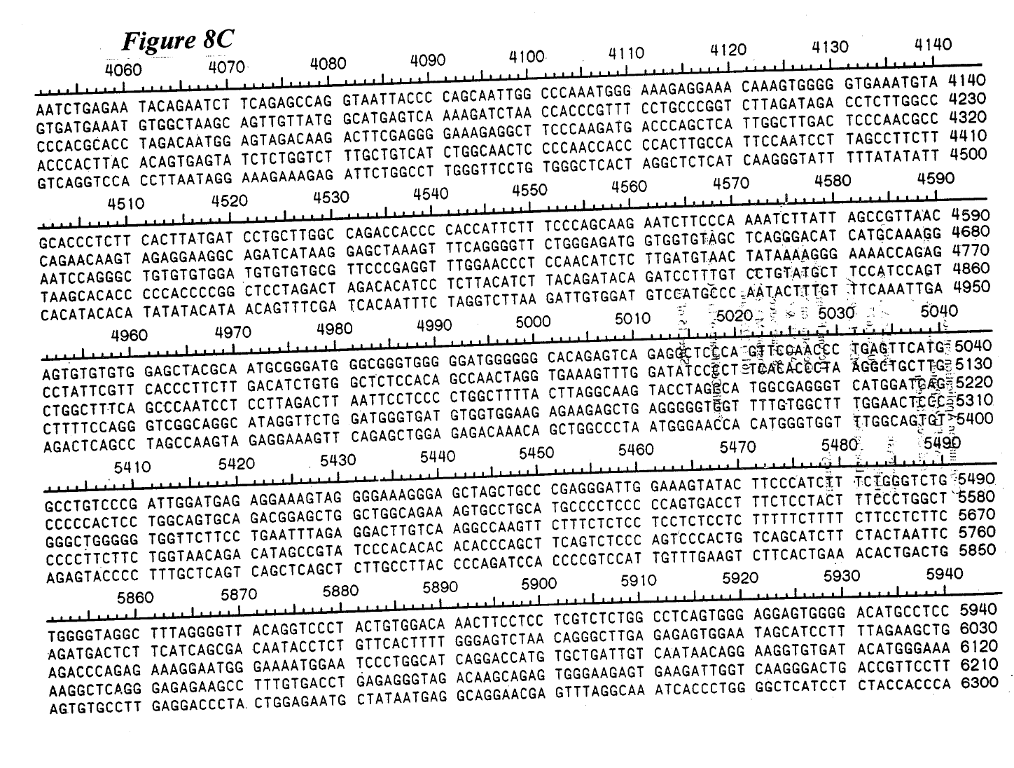

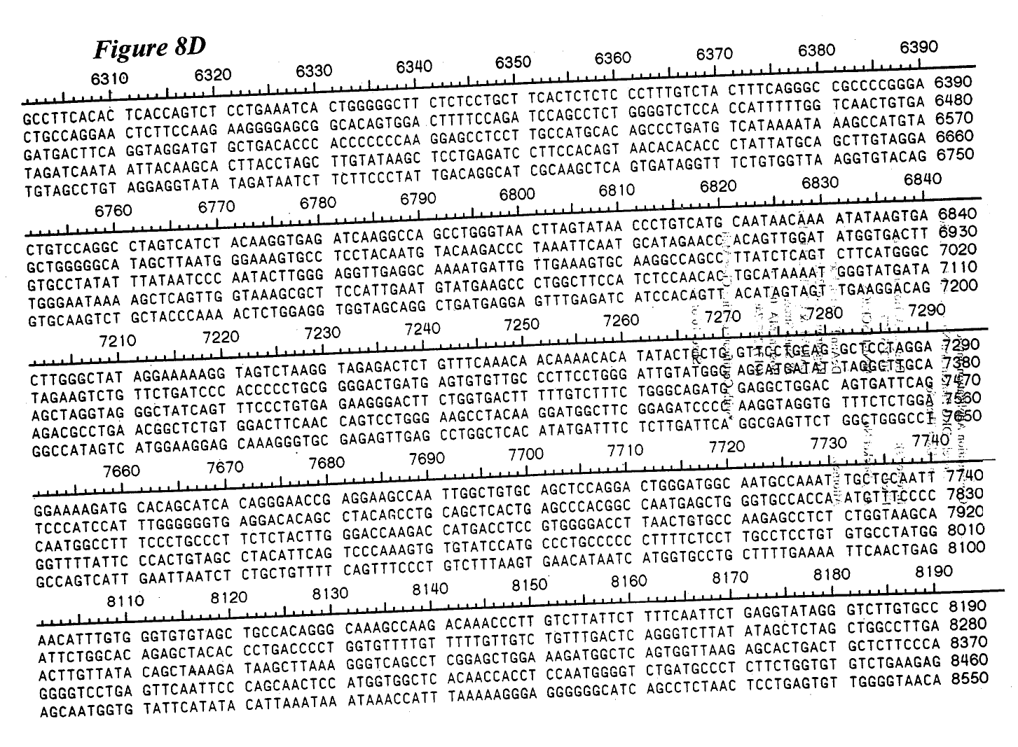

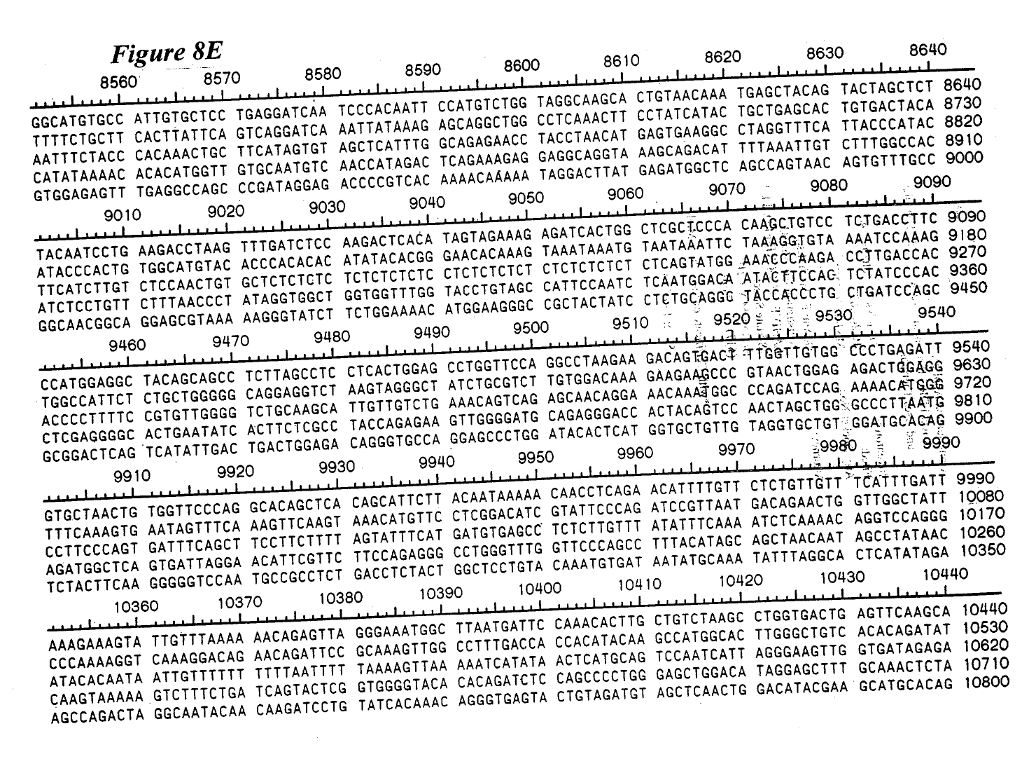

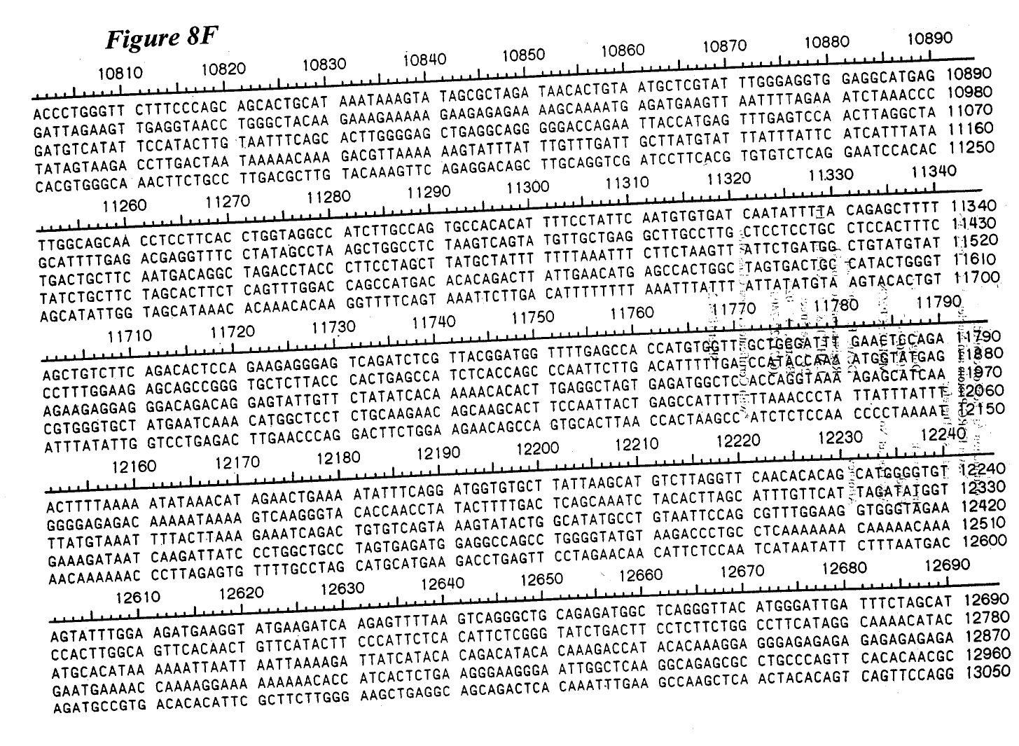

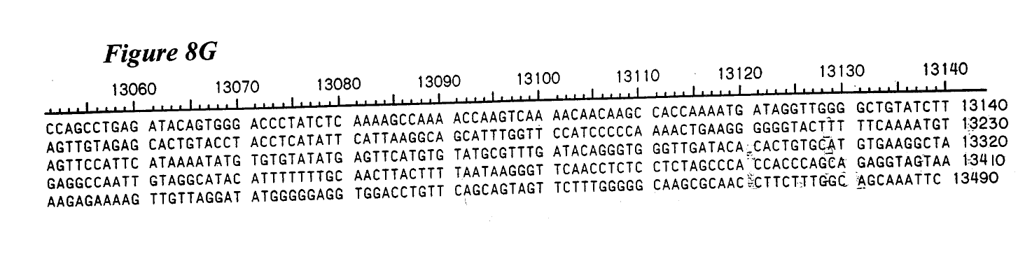

The genomic sequence of murine FDRG is set forth in FIG. 8 (SEQ ID NO:27). FIG. 1D depicts schematically the genomic FDRG sequence. Exons are denoted by black boxes and introns by a solid line. The mouse FDRG gene spans approximately 6 kb. The coding region is contained in seven exons, the last four of which encode the fibrinogen-like domain.

### Example 8

### Genetic Mapping of FDRG

The PGAR gene was localized to human chromosome 19 using the GENEBRIDGE4 Human/Hamster Radiation Hybrid Panel and the MapManager™ software. The human mapping primers used for PCR amplification were forward primer 5′-GATCGAGGCTGCAGGATATGC-3′ (SEQ ID NO:33) and reverse primer 5′-GTCAGTCAATGTGACTGAGTCC-3′ (SEQ ID NO:34). PCRs were performed with an annealing temperature of 52° C. and extension times of 50 s at 72° C. for 35 cycles, with a final extension of 5 min on an MJ Research Peltier PCT-225 Thermal Cycler. Amplification products were run on a 2% agarose gel, stained postelectrophoresis with SYBR Gold and scanned on a Molecular Dynamics Fluorimager 595. PCRs were also performed with mouse primers described above.

By radiation hybrid mapping, the human PGAR gene was localized to chromosome 19p13.3, 7.8 cR distal of marker AFMA135XB9 and 2.4 cR proximal of marker RP_S28_{—}1. This region is close to ATHS (atherosclerosis susceptibility) and ML4 (sialolipidosis) loci and syntenic to mouse chromosome 10.

### Example 9

### Characterization of Anti-Human FDRG Polyclonal Antibodies

A synthetic peptide corresponding to the C-terminus of human FDRG was prepared with the following sequence: KTWRGRYYPLQATTMLIQPMAA (SEQ ID NO:25). This was conjugated to KLH (keyhole limpet hemocyanin) at the N-terminal lysine and injected into two rabbits for the production of polyclonal antiserum using standard immunization protocols (e.g., Harlow and Lane, 1987).

A synthetic peptide corresponding to the N-terminus of human FDRG was prepared with the following sequence: PVQSKSPRFASWDEMN (SEQ ID NO:26). This was synthesized with a MAP core (Research Genetics, Rockville Md.) at the C-terminal asparagine. It was injected into two rabbits for the production of polyclonal antiserum using standard immunization protocols (e.g., Harlow and Lane, 1987).

Antiserum samples were harvested 10 weeks after the initial immunization. Immunoreactivity was tested by ELISA against the immunizing peptide and were positive as compared to pre-immune serum bleeds. Replicate samples of recombinant human FDRG:flag and mouse FDRG:HA protein supernatants and mock transfected negative control supernatants prepared as described above were run on SDS-PAGE and electroblotted onto PVDF membrane (Millipore). 1:1000 dilutions of all four rabbit antisera were used on replicate blots as primary antiserum followed by anti-rabbit HRP conjugated secondary antibodies. Blots were developed using chemiluminescent reagents (Renaissance, Dupont), and exposed to autoradiography film (Biomax MR2 film, Kodak).

Immunospecificity of the rabbit antisera were tested by standard Western blot methods. Antisera from the two rabbits immunized with the C-terminal peptide labeled a band in lanes containing human FDRG flag tagged at the C-terminus that was also immunoreactive with anti-flag M2 antibodies in replicate blots. The approximate molecular weight of this band was 40 (+/−5) kDa as determined using MultiMark standards (Novex). This band was not seen in lanes containing supernatant from mock or mouse FDRG HA transfected cells.

Antisera from rabbits immunized with the N-terminal peptide did not show any specific bands in lanes containing either human or mouse FDRG protein when compared to lanes containing mock transfected samples, indicating that by Western blot analysis these antibodies do not interact with FDRG protein.

### Example 10

### Tissue Expression of Murine FDRG

Total RNA was isolated from mouse liver, muscle, heart, spleen, white adipose, brown adipose, kidney, testes, brain, and lung tissue samples of 9 week old male C57BKFJ mice and analyzed for FDRG expression by northern blotting. The tissue distribution of FDRG mRNA is highly adipose tissue-selective, with at least 10-20 fold higher expression in white and brown fat over other tissues examined. Low levels of expression were detectable in other tissues (e.g., liver, kidney) and is believed to be due to fat contamination.

### Example 11

### In situ Hybridization of Murine Tissues

For in situ hybridization, fresh frozen sections (8 μm) of C57BL/6 mouse embryos and adult mouse tissues were prepared and treated for in situ analysis as previously described (Busfield et al. (1997) Mol. Cell. Biol. 17:4007-4014). A 548 bp fragment of the PGAR gene was PCR amplified using the primers 5′-GGAGCGGCACAGTGGACTTT-3′ (SEQ ID NO:28) and 5′-TACCCTTTTTACGCTCCTGC-3′ (SEQ ID NO:29) and used as the template for the synthesis of ³⁵S-labeled sense and antisense cRNA probes. Labeling and hybridization of probes were performed as described (Busfield et al., supra). White and brown adipose tissues from adult mouse were fixed in 10% formalin, paraffin embedded, and sectioned at 4 μm onto slides. Sections were deparaffinized in xylene, hydrated through a series of graded ethanols, and fixed in 4% formaldehyde/PBS for 10 minutes and rinsed twice in DEPC-PBS. After a 15 minute digestion with 20 μg/ml proteinase K, sections were immersed in 4% ethanol for 5 minutes, 0.2 N HCI for 10 minutes, rinsed in DEPC-PBS followed by a rinse in 0.1 M triethanolamine-HCl (pH 8.0). Subsequent treatments, including dehydration through alcohols and hybridization to radiolabeled probes, were similar to those of cryostat sections (Busfield et al., supra). Slides were dipped in nuclear tack emulsion (NTB-2: Eastman Kodak) and exposed for 60 days. After developing and fixation, slides were stained with hematoxylin and eosin-Y and coverslipped.

Analysis of expression on day E13.5 revealed a low level signal distributed widely in most organs and connective tissue with the noticeable exception of the CNS and the heart. By E15.5, however, a strong expression emerged in brown fat, which remained by far the highest expressing tissue until E18.5, the last time of examination before birth. Liver had the next highest signal at this stage. The expression in white adipose tissue, which develops postnatally in rodents, was analyzed by performing in situ hybridization on adult tissue. An intense, granular pattern of labeling is observed within the cytoplasm surrounding the lipid droplets, identifying the adipocytes as the major FDRG-producing cell type in this tissue.

### Example 12

### In vitro Regulation of Murine FDRG in PPARγ-Expressing Cells

NIH3T3 fibroblasts stably expressing PPARγ were treated with 10 μg/ml pioglitazone for 0, 2, and 4 hours. Northern blot analysis performed on the cellular RNA shows that FDRG mRNA is only barely detectable in untreated cells but undergoes at least a 10-fold increase within two hours after pioglitazone administration. Inhibition of protein synthesis by pre-treatment with 5 μg/ml cycloheximide does not block this induction. Cycloheximide by itself causes a rise in FDRG mRNA, a phenomenon often associated with immediate early genes and generally attributed to enhanced mRNA stability. Pretreatment with 5 μg/ml cycloheximide did not block PPARy ligand-produced induction but rather caused an additive increase. The persistence of TZD-mediated induction in the absence of protein synthesis, even after accounting for the effect of cycloheximide alone, is consistent with the notion of FDRG being a direct transcriptional target of PPARγ ligands, and is also in agreement with the rapid time course of its induction. The increases in FDRG message levels produced by pioglitazone and cycloheximide are additive, presumably reflecting independent mechanisms of regulation. These data demonstrate that FDRG is, in fact, regulated by known PPARγ activators.

Using the 3T3-L1 preadipocyte culture model of adipogenesis, the expression of FDRG was examined at various time points during hormone-induced differentiation. Briefly, 3T3-L1 preadipocytes were cultured to confluence and induced to differentiate using established protocols, ie. three days of dexamethasone/isobutylmethylxanthine/insulin treatment followed by insulin treatment. Northern analysis was performed on the total RNA isolated 3, 6, 9, and 12 days after induction (10 μg/lane). FDRG levels were found to increase dramatically in early differentiation, preceding well-known markers such as lipoprotein lipase, while lagging slightly behind PPARγ. The latter observation is compatible with FDRG being a target of PPARγ.

### Example 13

### In vivo Regulation of Murine FDRG in Zucker Diabetic Fatty Rats

This experiment describes analysis of FDRG mRNA levels in rats following chronic troglitazone treatment to determine if FDRG expresion is regulated by PPARγ in vivo.

Male Zucker diabetic fatty (fa/fa) rats were treated with 6 mg/kg/day (1.2%w/v) troglitazone or placebo admixed with food starting at 6 weeks of age and continuing until 26 weeks. By this time untreated littermates develop severe hyperglycemia and failure of the pancreatic γ-cell function, while the treated animals remain normoglycemic. Northern blots were performed on RNA isolated from white adipose tissue of treated and untreated animals, four from each group, after 4 and 20 weeks of treatment. FDRG expression in vivo was observed to be consistently upregulated 3-5 fold in the treated animals compared to the controls. This was true even when normalized to aP2, a fat cell marker. Glut4 mRNA was also found to be elevated, in agreement with published reports by other groups. Ribosomal-associated 36B4 served as a loading control.

### Example 14

### Regulation of FDRG in Murine Models of Obesity

FDRG expression was examined in white and brown fat tissues from 9 week old male obese (ob/ob) and diabetic (db/db) mice. Northern analysis showed uniform 3-4 fold elevation of FDRG mRNA in ob/ob and db/db animals compared to their respective congenic lean controls, both in white and brown fat. This may reflect a correlation of increased FDRG expression with obese states in general, raising the possibility that FDRG may contribute to some of the pathophysiological features of such states. Alternatively, FDRG mRNA levels may be regulated directly or indirectly by the genetic defects specific for these two models, i.e. leptin and the leptin receptor.

### Example 15

### Regulation of Murine FDRG by Nutrition and Leptin Administration

Possible effects of changes in nutritional states on expression of FDRG were investigated. In this study, lean mice were divided into three experimental groups, and were either allowed free access to food or subjected to 12 hours of fasting or 7.5 hours of fasting followed by refeeding (4.5 hours). The animals were sacrified at the end of 12 hours. Northern blot reveals a 2-3 fold increase in FDRG mRNA in white adipose tissue after a short-term fast, which is abrogated by refeeding. This suggests that FDRG levels are responsive to acute metabolic changes in vivo.

In a second study, FDRG expression in mice receiving intraperitoneal administration of leptin, with consequent decrease in dietary intake, was compared to that in mice diet restricted to match the amount of food intake by the former group. In this study, 10 week old lean or ob/ob mice were divided into three groups. The first group was the control, intraperitoneally injected with 1.5 ml PBS twice daily. The second group was injected with 12.5 mg/kg leptin in PBS twice daily, with consequent decrease in dietary intake. The third group, the pair-fed group, was diet restricted to match the amount of food intake by the leptin-injected group, and was also injected with PBS. The animals were sacrified at the end of one week. FDRG expression in brown fat was seen to go up substantially with restrictions of caloric intake, consistent with the findings in the first study, but this rise is not seen in leptin treated animals. That may reflect a fundamental difference in leptin-induced dietary restriction versus externally imposed restriction, or may be consistent with a further downstream effect, e.g., leptin-mediated suppression of FDRG upregulation that would ordinarily result from reduced food intake.

### Example 16

### Tissue Expression of Human FDRG

Northern blot analysis of RNA isolated from human placenta, heart, kidney, liver, and fat tissue samples showed high expression in white fat, in agreement with the murine FDRG tissue expression data, and in placenta. Placental expression is of interest because it is a tissue known to undergo extensive vascular remodelling in the adult (e.g., in its development during pregnancy) and involved in transport of metabolic substrates for the fetus. Another fat-derived secreted protein with important metabolic and endocrine functions, leptin, is also produced in abundant quantities in placenta.

### Example 17

### Expression of Recombinant FDRG Protein in Bacterial Cells

FDRG can be expressed as a recombinant glutathione-S-transferase (GST) fusion polypeptide in E. coli and the fusion polypeptide can be isolated and characterized. Specifically, FDRG is fused to GST and this fusion polypeptide is expressed in E. coli, e.g., strain PEB199. Expression of the GST-FDRG fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 18

### Expression of Recombinant FDRG Protein in COS Cells

To express the FDRG gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire FDRG protein and a HA tag (Wilson et al. (1984) Cell 37:767) fused in-frame to its 3′ end of the fragment is cloned into the polylinker region of the vector, thereby placing the expression of the recombinant protein under the control of the CMV promoter.

To construct the plasmid, the FDRG DNA sequence is amplified by PCR using two primers. The 5′ primer contains the restriction site of interest followed by approximately twenty nucleotides of the FDRG coding sequence starting from the initiation codon; the 3′ end sequence contains complementary sequences to the other restriction site of interest, a translation stop codon, the HA tag and the last 20 nucleotides of the FDRG coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the FDRG gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the FDRG-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the FDRG protein is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the FDRG coding sequence is cloned directly into the polylinker of the pcDNA/Amp vector using the appropriate restriction sites. The resulting plasmid is transfected into COS cells in the manner described above, and the expression of the FDRG protein is detected by radiolabelling and immunoprecipitation using a FDRG specific monoclonal antibody

### Example 19

### Retroviral Delivery of FDRG

The entire open reading frame of FDRG can be subcloned into the retroviral vector MSCVneo, described in Hawley et al. (1994) Gene Therapy 1: 136-138. Cells (293Ebna, Invitrogen) are then transiently transfected with the FDRG construct and with constructs containing viral regulatory elements, to produce high titre retrovirus containing the FDRG gene. The virus is then used to transfect mice. These mice are then tested for any gross pathology and for changes in biological response, e.g., cell migration, using standard assays.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
a) a nucleic acid molecule comprising a nucleotide sequence which is at least 60% homologous to a nucleotide sequence of SEQ ID NO:1, SEQ ID NO:3, SEQ ID NO:4, SEQ ID NO:11, SEQ ID NO:13, SEQ ID NO:14, the DNA insert of the plasmid deposited with ATCC as Accession Number 98632, or a complement thereof;
b) a nucleic acid molecule comprising a fragment of at least 555 nucleotides of a nucleic acid comprising the nucleotide sequence of SEQ ID NO:1, SEQ ID NO:3, SEQ ID NO:4, SEQ ID NO:11, SEQ ID NO:13, SEQ ID NO:14, the DNA insert of the plasmid deposited with ATCC as Accession Number 98632, or a complement thereof;
c) a nucleic acid molecule which encodes a polypeptide comprising an amino acid sequence at least about 60% homologous to the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632;
d) a nucleic acid molecule which encodes a fragment of a polypeptide comprising the amino acid sequence of SEQ ID NO:2, SEQ ID NO:5, SEQ ID NO:12, SEQ ID NO:15, or the polypeptide encoded by the DNA insert of the plasmid deposited with ATCC as Accession Number 98632, wherein the fragment comprises at least 15 contiguous amino acid residues of the amino acid sequence of SEQ ID NO:2, SEQ ID NO:5, SEQ ID NO:12, SEQ ID NO:15, or the polypeptide encoded by the DNA insert of the plasmid deposited with ATCC as Accession Number 98632; and
e) a nucleic acid molecule which encodes a naturally occurring allelic variant of a polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632, wherein the nucleic acid molecule hybridizes to a nucleic acid molecule comprising SEQ ID NO:1, SEQ ID NO:3, SEQ ID NO:4, SEQ ID NO:11, SEQ ID NO:13, or SEQ ID NO:14, under stringent conditions.

2. The isolated nucleic acid molecule of claim 1 which is selected from the group consisting of:
a) a nucleic acid molecule comprising the nucleotide sequence of SEQ ID NO: I, SEQ ID NO:3, SEQ ID NO:4, SEQ ID NO:11, SEQ ID NO:13, SEQ ID NO:14, or the DNA insert of the plasmid deposited with ATCC as Accession Number 98632, or a complement thereof; and
b) a nucleic acid molecule which encodes a polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2, SEQ ID NO:5, SEQ ID NO:12, SEQ ID NO:15, or the polypeptide encoded by the DNA insert of the plasmid deposited with ATCC as Accession Number 98632, wherein the fragment comprises at least 15 contiguous amino acids of SEQ ID NO:2, SEQ ID NO:5, SEQ ID NO:12, SEQ ID NO:15, or the amino acid sequence encoded by the DNA insert of the plasmid deposited with ATCC as Accession Number 98632;
b) a naturally occurring allelic variant of a polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632, wherein the polypeptide is encoded by a nucleic acid molecule which hybridizes to a nucleic acid molecule comprising SEQ ID NO:1, SEQ ID NO:3, SEQ ID NO:4, SEQ ID NO:11, SEQ ID NO:13, SEQ ID NO:14, under stringent conditions; and
c) a polypeptide which is encoded by a nucleic acid molecule comprising a nucleotide sequence which is at least 60% homologous to a nucleic acid comprising the nucleotide sequence of SEQ ID NO:1, SEQ ID NO:3, SEQ ID NO:4, SEQ ID NO:11, SEQ ID NO:13, SEQ ID NO:14, or the DNA insert of the plasmid deposited with ATCC as Accession Number 98632.
d) a polypeptide comprising an amino acid sequence which is at least 60% homologous to the amino acid sequence of SEQ ID NO:2, SEQ ID NO:5, SEQ ID NO:12, SEQ ID NO:15, or the polypeptide encoded by the DNA insert of the plasmid deposited with ATCC as Accession Number 98632.

9. The isolated polypeptide of claim 8 comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632;
b) a fragment of a polypeptide comprising the amino acid sequence of SEQ ID NO:2, SEQ ID NO:5, SEQ ID NO:12 or SEQ ID NO:15, or an amino acid sequence encoded by the DNA insert of the plasmid deposited with ATCC as Accession Number 98632 wherein the fragment comprises at least 15 contiguous amino acids of SEQ ID NO:2, SEQ ID NO:5, SEQ ID NO:12, SEQ ID NO:15, or the amino acid sequence encoded by the DNA insert of the plasmid deposited with ATCC as Accession Number 98632; and
c) a naturally occurring allelic variant of a polypeptide comprising the amino acid sequence of SEQ ID NO:2, SEQ ID NO:5, SEQ ID NO:12, SEQ ID NO:15, or an amino acid sequence encoded by the DNA insert of the plasmid deposited with ATCC as Accession Number 98632, wherein the polypeptide is encoded by a nucleic acid molecule which hybridizes to a nucleic acid molecule comprising SEQ ID NO:1, SEQ ID NO:3, SEQ ID NO:4, SEQ ID NO:11, SEQ ID NO:13, or SEQ ID NO:14, under stringent conditions;
comprising culturing the host cell of claim 5 under conditions in which the nucleic acid molecule is expressed.

13. A method for detecting the presence of a polypeptide of claim 8 in a sample comprising:
a) contacting the sample with a compound which selectively binds to the polypeptide; and
b) determining whether the compound binds to the polypeptide in the sample to thereby detect the presence of a polypeptide of claim 8 in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule in claim 1 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising:
a) contacting the polypeptide, or a cell expressing the polypeptide with a test compound; and
b) determining whether the polypeptide binds to the test compound.

20. The method of claim 19, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for FDRG activity.

21. A method of modulating the activity of a polypeptide of claim 8 comprising contacting the polypeptide or a cell expressing the polypeptide with a compound which binds to the polypeptide in a sufficient concentration to modulate the activity of the polypeptide.

22. A method for identifying a compound which modulates the activity of a polypeptide of claim 8 comprising:
a) contacting a polypeptide of claim 8 with a test compound; and
b) determining the effect of the test compound on the activity of the polypeptide to thereby identify a compound which modulates the activity of the polypeptide.

23. A method of treating obesity or diabetes in a subject comprising administering to the subject an agent selected from the group consisting of a small molecule modulator of FDRG, a FDRG nucleic acid molecule, a FDRG protein and a FDRG antibody such that obesity or diabetes in the subject is treated.

24. A method for diagnosing obesity or diabetes in a subject comprising:
a) isolating a sample from the subject;
b) contacting the sample with a nucleic acid probe or primer which selectively hybridizes to a FDRG nucleic acid molecule; and
c) determining whether the nucleic acid probe or primer binds to a FDRG nucleic acid molecule in the sample to thereby detect the presence of obesity or diabetes in the subject.

25. A method of for identifying a compound which modulates a metabolic disorder comprising:
a) contacting a cell which expresses a FDRG receptor with a compound to form an assay mixture:
b) contacting the assay mixture with FDRG;
c) determining whether the compound modulates FDRG activity, such that a modulatory compound is identified.

26. A method of regulating angiogenesis in a subject comprising administering to the subject an agent selected from the group consisting of a small molecule modulator of FDRG, a FDRG nucleic acid molecule, a FDRG protein and a FDRG antibody such that angiogenesis in the subject is regulated.


SEQUENCE LISTING (33 sequences)

SEQ ID NO: 1 - DNA, 1894 bp, Homo sapiens
  CDS (166)..(1383)
agtcgacccacgcgtccgcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgagggtccaccgaccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacaaaaaaaaaaaaaaagggcggccgc

SEQ ID NO: 2 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGPPTLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 3 - DNA, 1218 bp, Homo sapiens
  CDS (1)..(1218)
atgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgagggtccaccgaccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcc

SEQ ID NO: 4 - DNA, 1143 bp, Homo sapiens
  CDS (1)..(1143)
ggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgagggtccaccgaccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcc

SEQ ID NO: 5 - protein, 381 aa, Homo sapiens
GPVQSKSPRFASWDEMNVLAHGLLQLGQGLREHAERTRSQLSALERRLSACGSACQGTEGPPTLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 6 - DNA, 651 bp, Homo sapiens
  CDS (1)..(651)
ctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatg

SEQ ID NO: 7 - protein, 217 aa, Homo sapiens
LPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPM

SEQ ID NO: 8 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 9 - protein, 496 aa, Mus musculus
MWQIIFLTFGWDLVLASAYSNFRKSVDSTGRRQYQVQNGPCSYTFLLPETDSCRSSSSPYMSNAVQRDAPLDYDDSVQRLQVLENILENNTQWLMKLENYIQDNMKKEMVEIQQNVVQNQTAVMIEIGTSLLNQTAAQTRKLTDVEAQVLNQTTRLELQLLQHSISTNKLEKQILDQTSEINKLQNKNSFLEQKVLDMEGKHSEQLQSMKEQKDELQVLVSKQSSVIDELEKKLVTATVNNSLLQKQQHDLMETVNSLLTMMSSPNSKSSVAIRKEEQTTFRDCAEIFKSGLTTSGIYTLTFPNSTEEIKAYCDMDVGGGGWTVIQHREDGSVDFQRTWKEYKEGFGNPLGEYWLGNEFVSQLTGQHRYVLKIQLKDWEGNEAHSLYDHFYLAGEESNYRIHLTGLTGTAAKISSISQPGSDFSTKDSDNDKCICKCSQMLSGGWWFDACGPSNLNGQYYPQKQNTNKFNGIKWYYWKGSGYSLKATTMMIRPADF

SEQ ID NO: 10 - protein, 326 aa, Homo sapiens
MELSRVAVALGPTGQLLLFLSFQTLAAQAADTCPEVKVVGLEGSDKLSILRGCPGLPGAAGPKGEAGANGPKGERGSPGVVGKAGPAGPKGDRGEKGARGEKGEPGQLQSCATGPRTCKELLTRGHFLSGWHTIYLPDCQPLTVLCDMDTDGGGWTVFQRRSDGSVDFYRDWAAYKRGFGSQLGEFWLGNDHIHALTAQGTSELRVDLVDFEGNHQFAKYRSFQVAGEAEKYKLVLGGFLEGNAGDSLSSHRDQFFSTKDQDNDNHSGNCAEQYHGAWWYNACHSSNLNGRYLRGLHTSYANGVNWRSGRGYNYSYQVSEMKVRLT

SEQ ID NO: 11 - DNA, 1893 bp, Mus musculus
  CDS (149)..(1378)
gtcgacccacgcgtccggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggagctctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacaaaaaaaaaaaaaaaagggcggccgc

SEQ ID NO: 12 - protein, 410 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 13 - DNA, 1230 bp, Mus musculus
  CDS (1)..(1230)
atgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctct

SEQ ID NO: 14 - DNA, 1161 bp, Mus musculus
  CDS (1)..(1161)
caagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcagactcagctcaaggctcaaaacagcaagatccagcaattgttccagaaggtggcccagcagcagagatacctatcaaagcagaatctgagaatacagaatcttcagagccagatagacctcttggcccccacgcacctagacaatggagtagacaagacttcgaggggaaagaggcttcccaagatgacccagctcattggcttgactcccaacgccacccacttacacaggccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctct

SEQ ID NO: 15 - protein, 387 aa, Mus musculus
QGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAAS

SEQ ID NO: 16 - DNA, 651 bp, Mus musculus
  CDS (1)..(651)
ccgccccgggactgccaggaactcttccaagaaggggagcggcacagtggacttttccagatccagcctctggggtctccaccatttttggtcaactgtgagatgacttcagatggaggctggacagtgattcagagacgcctgaacggctctgtggacttcaaccagtcctgggaagcctacaaggatggcttcggagatccccaaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatg

SEQ ID NO: 17 - protein, 217 aa, Mus musculus
PPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPM

SEQ ID NO: 18 - DNA, 20 bp, synthetic construct
aattaaccctcactaaaggg

SEQ ID NO: 19 - DNA, 32 bp, synthetic construct
acgcgtcgactaatacgactcactatagggcg

SEQ ID NO: 20 - DNA, 62 bp, synthetic construct
tacgcgtcgacctaagcgtagtctgggacgtcgtatgggtaagaggctgctctagcctccat

SEQ ID NO: 21 - protein, 8 aa, synthetic construct
DYKDDDDK

SEQ ID NO: 22 - DNA, 35 bp, synthetic construct
tttttcaattgaccgccatgagcggtgctccgacg

SEQ ID NO: 23 - DNA, 59 bp, synthetic construct
tttttgtcgacttatcacttgtcgtcgtcgtccttgtagtcggaggctgcctctgctgc

SEQ ID NO: 24 - protein, 9 aa, synthetic construct
YPYDVPDYA

SEQ ID NO: 25 - protein, 22 aa, synthetic construct
KTWRGRYYPLQATTMLIQPMAA

SEQ ID NO: 26 - protein, 16 aa, synthetic construct
PVQSKSPRFASWDEMN

SEQ ID NO: 27 - DNA, 13499 bp, Murine sp.
agaacccacaaaaaatcagggcaggcatagtaacttcctgtaatctcagtactccaggagcaaggaatgggggatccctagggcaaggtggtgagcttcaggtccaagaatatttcttgccccagtaaatagagaaggacacttcgcttttccatacacatgcacacacaagagcatgctcacctgcccacgtgtgtgcccatacatatgttaatacacagcacacaaaaagctggtcatgttggcaaagtaattggtttcctttagtttttttaagactattttttattttgtatgtatgaatgcccccctggagatcccagaagagaactgatcccctggaactggagttgtagatggatgtgaatcaccacatgggttctcagatccaaacacaggtcttgtacaagaacagcaaatgtgtgcatgtgtctgtgtgtagctgtgtgcacacgtgtgcaggtgcctgagaaggccaagagggcactgggtcttccaaagctgaagactcagacagttgtgagcccagagtgggtgctgggaagcaaattcatttgctagggtatgcatgctcttaatcactaagccatctttctagtccctgtttctctctccctcctccatctattcatttatattgcttatgtgcatgccacagaaaactgttgaggtcagaggataattttggggagtcctttctctccttccaacatatgggttcatgaactgtagtcagatcatcagacttggaagcaagcatctttacccactgagtcatatgaccagccctaattttgttttgttttaatttatttattattatacataagtacattgtgtagctgtctttagacgcaccagaagagggcatcagattttgttacgggtggttgtgagccaccatgtagttactgggatttgaactcagacctttggaagagcagtcagtgctcttgcctgctgagccatctcaccagccctattttgtttctcttacaggtggaccttcttgctactatcctctaagtgctaagatgacaggcatgcaccaccacacccatcttggtggtttttctctggaggcaagaagacttggacaaggagatggcacatggactagcatggaatggctatgagctgtgcccaaaggctgatgtttggagagaacagatggtggatgtatgaattcatcccttgttgagggttttctgcctactcttcctgtctgcctgtcactgctgctgggatcagcctggcaaaaggcccagctggtcaagtgtacaagcagtggcctgcatgtcagccttgaggcatgcagtttcagattccagaggtttgctgctggctcccagccctgctgactcaacttgggggtttttcagtcaatttgccctaagggtcttcttgtcagacaggttttctgtgccaggcttcaggggaactgtcagaaaaaggaacaccaatgcaaacttgttgaataaaaacttaaccaggcagcataaacaaggaaagctgtctgccccaccccagtcctcttttgtctctgccttgtccctctcccctgctcaaaggtaggatttatacacaaactaaaaaactcagtacaggatctaggctgtatggaatgagtgaatgaatggatgggtgtggaagggaaatctttgctgatgttctctagattgtttgctttttaaattttttttaaaaaagatgtatttattttatttatatgagtacattgtagctgtcttcagacacaccggaagagggtatcagatcctattacagatggttgtaagccaccatgtggttgctgggaattaactcaggacctttggaagagcagtcagtactcttaactgctgaaccatctctccagcctgctttttaaatgtttaaatgactgttttaaattgtgtgcatgtgtgtgagtgcctgaggaggtcaaagtcaccatagctcctggagctggaattataggtgactgtgagctgttggacttagatcccctgcaaaagatgtaaatgctcttaactggtaagatagatttccagttcctcctggcttttttagttgttgttgttgttgttttgtgacaaggtcttcttcctggatggcctggaacttcactatgtagaccaggctgacctcaaactcacagagcttcctccaattgcttcttgctctgcagtgctgggagttaaagtatgcataactatgaattgtttttgttttgttttgaaacaaggtcttgctgtttcagcgttgactggtctgtaacttactatgtagagcaggctagcttgtagcttgtactattcccctgtgtcctgcgtgatgggtttacaggtgcgtgccaccagttttgttgttgctgttgttgtttggagacctggctcagagtagcccaggctctcttaaactcacttgtgtagctcaagatgaattccacactcttctgctccatgccccaagagctgggatgacaggtctgtcctcagacactgatcctcctttaacgcaggtaaattcctggacaccaagacccctggtggtggaggacaggcaacaggaggaagcctagggttgcaatctctacaatgtagacagaaatcccagctattgactatttggccaggaagaggtgactaattgaacaagggtgttagtgatcctcacaaacacatctgcaatgacgaatgcttgagctggaaagagcgtgggagccagaaaggtggtggaagtgagagtgggggcaggggttccgcctggtctgtggatttaagatctcaggaagcctctaagaggcagagagcaaggttctgtaatcccccacttgccatctgaactcagcctaccagggagagaactttctgccacgtaactctgccctactttgcttttgacttgagaaggggtcagtcaagaaggcatccacgtagcgggaaggggaaaaaaaaaaaagaaggcatctacgtcctgatttgtacgccctttcctccattgaaccgggctgggatgcgggtagggctgggaatttccggccttaggatgaaccatgcagtgcacagacagtcacgtcgcttattaggtcgcaaggaaggcgaagcagccatccccaagaactcttcatacacacacacacacacacacacacacacacacacacacacgccacacacacacactcgtaactacaaagcctgtggcattgcaccctaaaacttggccactcgctccgcccccgggccccgcctccaatgctctccctcccactcccacacccgctttataaagtggggctttaggtgcaaccgtgaaacggcttatgagctacgggctccagatcttcttctgcaccagagcaagtctaagtctgagccggctcccccagaactccagctgctgggtcttgaactcctgcgttccggagtcctagcgttgctgcacccaaggccacccccagaatcatgcgctgcgctccgacagcaggcgctgccctggtgctatgcgcggctactgcggggcttttgagcgcgcaagggcgccctgcacagccagagccaccgcgctttgcatcctgggacgagatgaacttgctggctcacgggctgctacagctcggccatgggctgcgcgaacacgtggagcgcacccgtgggcagctgggcgcgctggagcgccgcatggctgcctgtggtaacgcttgtcaggggcccaagggaaaagatgcacccttcaaagactccgaggatagagtccctgaaggccagactcctgagactctgcagagtttgcaggtaggcgcatctactaggactctgactctacttagactaacgagtctagtactccaggacctgggtggagaagggcctcagagcagactgtgaaagggtagaagggagggtgaataagaggaggttgccgacataaagccaggtcttcactagccttgcatatcttcagactcagctcaaggctcaaaacagcaagatccagcaattgttccagaaggtggcccagcagcagagatacctatcaaagcagaatctgagaatacagaatcttcagagccaggtaattaccccagcaattggcccaaatgggaaagaggaaacaaagtgggggtgaaatgtagtgatgaaatgtggctaagcagttgttatggcatgagtcaaaagatctaaccacccgtttcctgcccggtcttagatagacctcttggcccccacgcacctagacaatggagtagacaagacttcgaggggaaagaggcttcccaagatgacccagctcattggcttgactcccaacgccacccacttacacagtgagtatctctggtctttgctgtcatctggcaactccccaaccaccccacttgccattccaatccttagccttcttgtcaggtccaccttaataggaaagaaagagattctggccttgggttcctgtgggctcactaggctctcatcaagggtatttttatatattgcaccctcttcacttatgatcctgcttggccagaccaccccaccattctttcccagcaagaatcttcccaaaatcttattagccgttaaccagaacaagtagaggaaggcagatcataaggagctaaagtttcaggggttctgggagatggtggtgtagctcagggacatcatgcaaaggaatccagggctgtgtgtggatgtgtgtgcgttcccgaggtttggaaccctccaacatctcttgatgtaactataaaagggaaaaccagagtaagcacacccccaccccggctcctagactagacacatcctcttacatcttacagatacagatcctttgtcctgtatgcttccatccagtcacatacacatatatacataacagtttcgatcacaatttctaggtcttaagattgtggatgtccatgcccaatactttgtttcaaattgaagtgtgtgtggagctacgcaatgcgggatgggcgggtgggggatggggggcacagagtcagaggctcccagttccaaccctgagttcatgcctattcgttcacccttcttgacatctgtggctctccacagccaactaggtgaaagtttggatatccccttcacaccctaaggctgcttgctggctttcagcccaatcctccttagacttaattcctcccctggcttttacttaggcaagtacctaggcatggcgagggtcatggatgagcttttccagggtcggcaggcataggttctggatgggtgatgtggtggaagagaagagctgagggggtggttttgtggctttggaactcccagactcagcctagccaagtagaggaaagttcagagctggagagacaaacagctggccctaatgggaaccacatgggtggtttggcagtgtgcctgtcccgattggatgagaggaaagtaggggaaagggagctagctgcccgagggattggaaagtatacttcccatctttctgggtctgcccccactcctggcagtgcagacggagctggctggcagaaagtgcctgcatgcccctcccccagtgacctttctcctactttccctggctgggctgggggtggttcttcctgaatttagaggacttgtcaaggccaagttctttctctcctcctctcctctttttcttttcttcctcttcccccttcttctggtaacagacatagccgtatcccacacacacacccagcttcagtctcccagtcccactgtcagcatcttctactaattcagagtacccctttgctcagtcagctcagctcttgccttaccccagatccaccccgtccattgtttgaagtcttcactgaaacactgactgtggggtaggctttaggggttacaggtccctactgtggacaaacttcctcctcgtctctggcctcagtgggaggagtggggacatgcctccagatgactcttcatcagcgacaatacctctgttcacttttgggagtctaacagggcttgagagagtggaatagcatccttttagaagctgagacccagagaaaggaatgggaaaatggaatccctggcatcaggaccatgtgctgattgtcaataacaggaaggtgtgatacatgggaaaaaggctcagggagagaagcctttgtgacctgagagggtagacaagcagagtgggaagagtgaagattggtcaagggactgaccgttccttagtgtgccttgaggaccctactggagaatgctataatgaggcaggaacgagtttaggcaaatcaccctggggctcatcctctaccacccagccttcacactcaccagtctcctgaaatcactgggggcttctctcctgcttcactctctccctttgtctactttcagggccgccccgggactgccaggaactcttccaagaaggggagcggcacagtggacttttccagatccagcctctggggtctccaccatttttggtcaactgtgagatgacttcaggtaggatgtgctgacacccacccccccaaggagcctccttgccatgcacagccctgatgtcataaaataaagccatgtatagatcaataattacaagcacttacctagcttgtataagctcctgagatccttccacagtaacacacaccctattatgcagcttgtaggatgtagcctgtaggaggtatatagataatcttcttccctattgacaggcatcgcaagctcagtgataggtttctgtggttaaggtgtacagctgtccaggcctagtcatctacaaggtgagatcaaggccagcctgggtaacttagtataaccctgtcatgcaataacaaaatataagtgagctgggggcatagcttaatgggaaagtgcctcctacaatgtacaagaccctaaattcaatgcatagaaccacagttggatatggtgacttgtgcctatatttataatcccaatacttgggaggttgaggcaaaatgattgttgaaagtgcaaggccagccttatctcagtcttcatgggctgggaataaaagctcagttggtaaagcgcttccattgaatgtatgaagccctggcttccatctccaacactgcataaaatgggtatgatagtgcaagtctgctacccaaaactctggaggtggtagcaggctgatgaggagtttgagatcatccacagttacatagtagttgaaggacagcttgggctataggaaaaaggtagtctaaggtagagactctgtttcaaacaacaaaacacatatactcctggttcctgcaggctcctaggatagaagtctgttctgatcccaccccctgcggggactgatgagtgtgttgcccttcctgggattgtatgggagcatgatattaggcttgcaagctaggtagggctatcagtttccctgtgagaagggacttctggtgactttttgtctttctgggcagatggaggctggacagtgattcagagacgcctgaacggctctgtggacttcaaccagtcctgggaagcctacaaggatggcttcggagatccccaaggtaggtgtttctctggaggccatagtcatggaaggagcaaagggtgcgagagttgagcctggctcacatatgatttctcttgattcaggcgagttctggctgggcctggaaaagatgcacagcatcacagggaaccgaggaagccaattggctgtgcagctccaggactgggatggcaatgccaaattgctccaatttcccatccatttggggggtgaggacacagcctacagcctgcagctcactgagcccacggccaatgagctgggtgccaccaatgtttcccccaatggcctttccctgcccttctctacttgggaccaagaccatgacctccgtggggaccttaactgtgccaagagcctctctggtaagcaggttttattcccactgtagcctacattcagtcccaaagtgtgtatccatgccctgccccccttttctccttgcctcctgtgtgcctatgggccagtcattgaattaatctctgctgttttcagtttccctgtctttaagtgaacataatcatggtgcctgcttttgaaaattcaactgagaacatttgtgggtgtgtagctgccacagggcaaagccaagacaaacccttgtcttattcttttcaattctgaggtatagggtcttgtgccattctggcacagagctacaccctgacccctggtgttttgtttttgttgtctgtttgactcagggtcttatatagctctagctggccttgaacttgttatacagctaaagataagcttaaagggtcagcctcggagctggaaagatggctcagtggttaagagcactgactgctcttcccaggggtcctgagttcaattcccagcaactccatggtggctcacaaccacctccaatggggtctgatgccctcttctggtgtgtctgaagagagcaatggtgtattcatatacattaaataaataaaccatttaaaaagggaggggggcatcagcctctaactcctgagtgttggggtaacaggcatgtgccattgtgctcctgaggatcaatcccacaattccatgtctggtaggcaagcactgtaacaaatgagctacagtactagctctttttctgcttcacttattcagtcaggatcaaattataaagagcaggctggcctcaaacttcctatcatactgctgagcactgtgactacaaatttctacccacaaactgcttcatagtgtagctcatttggcagagaacctacctaacatgagtgaaggcctaggtttcattacccataccatataaaacacacatggttgtgcaatgtcaaccatagactcagaaagaggaggcaggtaaagcagacattttaaattgtctttggccacgtggagagtttgaggccagcccgataggagaccccgtcacaaaacaaaaataggacttatgagatggctcagccagtaacagtgtttgcctacaatcctgaagacctaagtttgatctccaagactcacatagtagaaagagatcactggctcgctcccacaagctgtcctctgaccttcatacccactgtggcatgtacacccacacacatatacacgggaacacaaagtaaataaatgtaataaattctaaaggtgtaaaatccaaagttcatcttgtctccaactgtgctctctctctctctctctcctctctctctctctctctctctcagtatggaaacccaagaccttgaccacatctcctgttctttaaccctataggtggctggtggtttggtacctgtagccattccaatctcaatggacaatacttccactctatcccacggcaacggcaggagcgtaaaaagggtatcttctggaaaacatggaagggccgctactatcctctgcaggctaccaccctgctgatccagcccatggaggctacagcagcctcttagcctcctcactggagcctggttccaggcctaagaagacagtgactttggttgtggccctgagatttggccattctctgctgggggcaggaggtctaagtagggctatctgcgtcttgtggacaaagaagaagcccgtaactggagagactggaggaccccttttccgtgttggggtctgcaagcattgttgtctgaaacagtcagagcaacaggaaacaaatggcccagatccagaaaacatgggctcgaggggcactgaatatcacttctcgcctaccagagaagttggggatgcagagggaccactacagtccaactagctgggcccttaatggcggactcagtcatattgactgactggagacagggtgccaggagccctggatacactcatggtgctgttgtaggtgctgtggatgcacaggtgctaactgtggttcccaggcacagctcacagcattcttacaataaaaacaacctcagaacattttgttctctgttgtttcatttgatttttcaaagtgaatagtttcaaagttcaagtaaacatgttcctcggacatcgtattcccagatccgttaatgacagaactggttggctattccttcccagtgatttcagcttccttcttttagtatttcatgatgtgagcctctcttgtttatatttcaaaatctcaaaacaggtccagggagatggctcagtgattaggaacattcgttcttccagagggcctgggtttggttcccagcctttacatagcagctaacaatagcctataactctacttcaagggggtccaatgccgcctctgacctctactggctcctgtacaaatgtgataatatgcaaatatttaggcactcatatagaaaagaaagtattgtttaaaaaacagagttagggaaatggcttaatgattccaaacacttgctgtctaagcctggtgactgagttcaagcacccaaaaggtcaaaggacagaacagattccgcaaagttggcctttgaccaccacatacaagccatggcacttgggctgtcacacagatatatacacaataattgttttttttttaatttttaaaagttaaaaatcatataactcatgcagtccaatcattagggaagttggtgatagagacaagtaaaaagtctttctgatcagtactcggtggggtacacacagatctccagcccctgggagctggacataggagctttgcaaactctaagccagactaggcaatacaacaagatcctgtatcacaaacagggtgagtactgtagatgtagctcaactggacatacgaagcatgcacagaccctgggttctttcccagcagcactgcataaataaagtatagcgctagataacactgtaatgctcgtatttgggaggtggaggcatgaggattagaagttgaggtaacctgggctacaagaaagaaaaagaagagagaaaagcaaaatgagatgaagttaattttagaaatctaaacccgatgtcatattccatacttgtaatttcagcacttggggagctgaggcagggggaccagaattaccatgagtttgagtccaacttaggctatatagtaagaccttgactaataaaaacaaagacgttaaaaaagtatttatttgtttgattgcttatgtatttatttattcatcatttatacacgtgggcaaacttctgccttgacgcttgtacaaagttcagaggacagcttgcaggtcgatccttcacgtgtgtctcaggaatccacacttggcagcaacctccttcacctggtaggccatcttgccagtgccacacattttcctattcaatgtgtgatcaatattttacagagcttttgcattttgagacgaggtttcctatagcctaagctggcctctaagtcagtatgttgctgaggcttgccttgctcctcctgcctccactttctgactgcttcaatgacaggctagacctacccttcctagcttatgctatttttttaaatttcttctaagttattctgatggctgtatgtattatctgcttctagcacttctcagtttggaccagccatgacacacagacttattgaacatgagccactggctagtgactgccatactgggtagcatattggtagcataaacacaaacacaaggttttcagtaaattcttgacattttttttaaatttatttattatatgtaagtacactgtagctgtcttcagacactccagaagagggagtcagatctcgttacggatggttttgagccaccatgtggttgctgggatttgaactccagacctttggaagagcagccgggtgctcttacccactgagccatctcaccagcccaattcttgacatttttgaccataccaaaatggtatgagagaagaggagggacagacaggagtattgttctatatcacaaaaacacacttgaggctagtgagatggctcaccaggtaaaagagcatcaacgtgggtgctatgaatcaaacatggctcctctgcaagaacagcaagcacttccaattactgagccattttttaaaccctattatttatttatttatattggtcctgagacttgaacccaggacttctggaagaacagccagtgcacttaaccactaagccatctctccaacccctaaaatacttttaaaaatataaacatagaactgaaaatatttcaggatggtgtgcttattaagcatgtcttaggttcaacacacagcatggggtgtggggagagacaaaaataaaagtcaagggtacaccaacctatacttttgactcagcaaatctacacttagcatttgttcattagatatggtttatgtaaattttacttaaagaaatcagactgtgtcagtaaagtatactggcatatgcctgtaattccagcgtttggaaggtgggtagaagaaagataatcaagattatccctggctgcctagtgagatggaggccagcctggggtatgtaagaccctgcctcaaaaaaacaaaaacaaaaacaaaaaacccttagagtgttttgcctagcatgcatgaagacctgagttcctagaacaacattctccaatcataatattctttaatgacagtatttggaagatgaaggtatgaagatcaagagttttaagtcagggctgcagagatggctcagggttacatgggattgatttctagcatccacttggcagttcacaactgttcatacttcccattctcacattctcgggtatctgacttcctcttctggccttcataggcaaaacatacatgcacataaaaaattaattaattaaaagattatcatacacagacatacacaaagaccatacacaaaggagggagagagagagagagagagaatgaaaaccaaaaggaaaaaaaaacaccatcactctgaagggaagggaattggctcaaggcagagcgcctgcccagttcacacaacgcagatgccgtgacacacattcgcttcttgggaagctgaggcagcagactcacaaatttgaagccaagctcaactacacagtcagttccaggccagcctgagatacagtgggaccctatctcaaaagccaaaaccaagtcaaaacaacaagccaccaaaatgataggttggggctgtatcttagttgtagagcactgtacctacctcatattcattaaggcagcatttggttccatcccccaaaactgaaggggggtactttttcaaaatgtagttccattcataaaatatgtgtgtatatgagttcatgtgtatgcgtttgatacagggtgggttgatacacactgtgcatgtgaaggctagaggccaattgtaggcatacatttttttgcaacttacttttaataagggttcaacctctcctctagcccaccacccagcagaggtagtaaaagagaaaagttgttaggatatgggggaggtggacctgttcagcagtagttctttgggggcaagcgcaaccttctttggcagcaaattc

SEQ ID NO: 28 - DNA, 20 bp, Artificial Sequence
  Description of Artificial Sequence primer
ggagcggcacagtggacttt

SEQ ID NO: 29 - DNA, 20 bp, Artificial Sequence
  Description of Artificial Sequence primer
taccctttttacgctcctgc

SEQ ID NO: 30 - DNA, 21 bp, Artificial Sequence
  Description of Artificial Sequence primer
ctcttagcctcctcactggag

SEQ ID NO: 31 - DNA, 21 bp, Artificial Sequence
  Description of Artificial Sequence primer
cacagttagcacctgtgcatc

SEQ ID NO: 32 - DNA, 21 bp, Artificial Sequence
  Description of Artificial Sequence primer
gatcgaggctgcaggatatgc

SEQ ID NO: 33 - DNA, 22 bp, Artificial Sequence
  Description of Artificial Sequence primer
gtcagtcaatgtgactgagtcc